(19) United States
(12) Patent Application Publication
(10) Pub. No.: US 2024/0016813 A1
(43) Pub. Date: Jan. 18, 2024

(54) METHODS OF TREATING SYMPTOMS OF CORONAVIRUS INFECTION

(71) Applicant: Accencio LLC, Philadelphia, PA (US)

(72) Inventors: Kevin Brown, Philadelphia, PA (US); Kevin Brogle, Cream Ridge, NJ (US)

(21) Appl. No.: 18/044,964

(22) PCT Filed: Sep. 9, 2021
(86) PCT No.: PCT/US2021/049647

§ 371 Date: Mar. 10, 2023

Related U.S. Application Data
(60) Provisional application No. 63/076,806, filed on Sep. 10, 2020.
(60) Provisional application No. 63/076,821, filed on Sep. 10, 2020.
(60) Provisional application No. 63/077,870, filed on Sep. 14, 2020.
(60) Provisional application No. 63/077,883, filed on Sep. 14, 2020.

(51) Int. Cl. A61K 31/55; A61K 31/454

(52) U.S. Cl. CPC A61K 31/55; A61K 31/454

(57) ABSTRACT

The present disclosure relates to methods of treating at least one symptom of a coronavirus infection, or preventing an acute inflammatory response, e.g., a cytokine storm in a coronavirus patient, in particular a SARS-CoV-19 patient, by administering an angiontensin converting enzyme-2 (ACE2) modulator.

### CROSS REFERENCE TO RELATED APPLICATIONS

The present application claims priority to and the benefit of U.S. patent application No. 63/076,806, filed Sep. 10, 2020, US patent application No. 63/076,821, filed Sep. 10, 2020, US patent application No. 63/077,870, filed Sep. 14, 2020, U.S. patent application No. 63/077,883, filed Sep. 14, 2020, each of which is hereby incorporated by reference in its entirety.

### FIELD

The present disclosure relates to methods of treating one or more symptoms of a coronavirus infection, particularly SARS-CoV-19. The present disclosure further relates to methods of treating or preventing an acute inflammatory response, e.g., a cytokine storm in a coronavirus patient, by administering a modulator of angiotensin converting enzyme-2 (ACE2). The present disclosure also relates to methods of treating symptoms of a coronavirus infection, e.g., SARS-CoV-19, by administering an inhibitor of interleukin-6 (IL-6). The present disclosure further relates to methods of treating symptoms of a coronavirus infection, e.g., SARS-COV-19, by administering a CXC chemokine receptor 3 (CXCR3) and/or CXC chemokine receptor 4 (CXCR4) antagonist. The present disclosure also relates to methods of treating symptoms of a coronavirus infection, e.g., SARS-CoV49. by administering a Toll-Like-Receptor (TLR) antagonist, in particular a TLR-7 or TLR-8 antagonist.

### BACKGROUND

The novel virus 2019-nCoV (SARS-CoV-19, COVID-19), is the third well-known coronavirus to cross species to infect human populations in the past two decades. The previous two are the severe acute respiratory syndrome coronavirus (SARS-CoV) outbreak in 2002 and the Middle East respiratory syndrome coronavirus (MFRS-CoV) outbreak in 2012. Like SARS-CoV and MERs-CoV, SARS-CoV-19 causes severe respiratory illness, and is highly transmissible from human-to-human. On March ii, 2020, the World Health Organization (WHO) declared SARS-CoV-19 a global pandemic. Since then, over 20 million people have been infected, and over 750,000 people have died worldwide from the virus. In the United States alone there have been over 5 million infections to date, with over 160,000 deaths.

Most of the critically ill patients do not develop severe clinical manifestations in early stages of the diseases; however, these patients rapidly deteriorate in the later stages of the disease, presenting with Acute Respiratory Distress Syndrome (ARDS) and multiple-organ failure, resulting in death within a short time. Evidence suggests that proinflammatory responses play a role in the pathogenesis of SARS-CoV-19 and other coronaviruses. Dysregulations of cytokine-chemokine responses cause the immune system to become hyperactive and induce a condition called a cytokine storm, which is considered to be one of the major causes of ARDS and multiple-organ failure in these patients. Targeting cytokines during the management of SARS-CoV-19 patients could improve survival rates and reduce mortality.

### SUMMARY

The present disclosure relates to methods of treating one or more symptoms of a coronavirus infection, particularly SARS-CoV-19. The present disclosure further relates to methods of treating or preventing an acute inflammatory response, e.g., a cytokine storm in a coronavirus patient, by administering a modulator of angiotensin converting enzyme-2 (ACE2).

The coronaviruses SARS-CoV and SARS-COV-19 mediate their host cell entry via attachment to ACE2-membrane receptors. SARS-CoV-19 enters the lungs, where the spike glycoprotein of the virus binds to ACE2 on cells, allowing the virus to enter the cells. The present disclosure is based on the discovery that ACE2 is a therapeutic target for the treatment of coronavirus symptoms, in particular in reducing inflammation and preventing cytokine storms in patients with coronavirus infections, in particular SARS-CoV-19.

Thus, in some embodiments, the present disclosure relates to a method of treating or alleviating at least one symptom of a coronavirus infection in a subject, by administering to the subject a therapeutically effective amount of an ACE2 modulator. In some embodiments, the ACE2 modulator is an ACE2 inhibitor. In some embodiments, the symptom is selected from the group consisting of fever, cough, tiredness, sore throat, diarrhea, conjunctivitis, headache, loss of taste, loss of smell, rash, difficulty breathing, shortness of breath, chest pain, chest pressure, Acute Respiratory Distress Syndrome (ARDS) and organ failure. In some embodiments, the subject is a human.

In some embodiments, the present disclosure relates to a method of treating an acute inflammatory condition in a subject infected with a coronavirus, the method comprising the step of administering to the subject a therapeutically effective amount of an angiotensin converting-2 (ACE2) modulator, In some embodiments, the ACE2 modulator is an ACE2 inhibitor. In some embodiments, the inflammatory condition comprises a cytokine storm. In some embodiments, the subject is a human.

In some embodiments, the present disclosure relates to a method of preventing a cytokine storm in a subject infected with a coronavirus, the method comprising the step of administering to the subject a therapeutically effective amount of an angiotensin converting-2 (ACE2) modulator. In some embodiments, the ACE2 modulator is an ACE2 inhibitor. In some embodiments, the subject is a human.

In some embodiments, the present disclosure relates to a method of reducing or arresting viral load in a subject infected with a coronavirus, the method comprising the step of administering to the subject a therapeutically effective amount of an angiotensin converting-2 (ACE2) modulator. In some embodiments, the ACE2 modulator is an ACE2 inhibitor. In some embodiments, the subject is a human.

In some embodiments, the coronavirus is a severe acute respiratory syndrome coronavirus (SARS-CoV). In some embodiments, the coronavirus is a novel virus 2019-nCoV (SARS-CoV-19). In some embodiments, the coronavirus is a Middle East respiratory syndrome coronavirus (MERS-CoV). In one preferred embodiment, the coronavirus is SARS-CoV-19.

In some embodiments the ACE2 modulator is selected from the group consisting of (S,S)-2-(1-Carboxy-2-(3-(3,5-dichlorobenzyl)-3H-imidazol-4-yl)-ethylamino)-4-methylpentanoic acid (MLN-4760); (2S)-2-acetamido-3-phenylpropanoic acid, or N-Acetyl-DL-phenylalanine (MR708); (2S)-2-[[2S,3R)-3-amino-2-hydroxy-4-phenylbutanoyl]amino]-4-methylpentanoic acid (Ubenimex); (S)-2-amino-3-(4-boronophenyl)propartoic acid (Borofalan (10B); (2-phenylacetyl)-L-glutamine (Antineoplaston AS2-5); 124-I-4-iodo-phenylalanine (124-I-TLX-101); 131-I-4-iodo-phenylalanine (131-1-TLX- 101); ethyl (2S)-2-[[2-(acetylsulfanylmethyl)-3-(2-methylphenyl)propanoyl]amino]-4-methylsulfanylbutanoate (SGH-42495); (((S)-1-carboxy-5-((4-iodobenzyl)amino) pentyl)carbamoyl)-L-glutamic acid (Iofolastat-I 123); 8-guanidino-octanoyl-Asp-Phe (SC-49992); (S)-5-amino-2-((1-propyl-1H-imidazol-4-yl)methyl)pentanoic acid (UK-369082); 4-{[2-(1H-imidazol-4-yl)ethyl]carbamoyl}butanoic acid (Ingavirin®); 4-hydroxy-N,1-dimethyl-2-oxo-N-phenyl-3-quinolinecarboxamide (Roquinimex); 5-chloro-N-ethyl-4-hydroxy-1-methyl-2-oxo-N-phenyl-1,2-dihydroquinoline-3-carboxamide (Laquinimod); 4-hydroxy-5-methoxy-N,1-dimethyl-2-oxo-N-[4-(trifluoromethyl)phenyl]quinoline-3 -carboxamide (Tasquinimod); Acetyl-L-leucine; (−m)-N-[(trans-4-isopropylcyclohexane)carbonyl]-D-phenylalanine (Nateglinide); S-(((R)-2-acetamido-2-carboxyethyl)thio)-N-acetyl-D-cysteine (N,N′-diacetyl-L-cystine); 2-Hydroxy-5-[(7-hydroxy-8-triethyl-6-nitro-2-oxochromerie-3-carbonyl)amino]benzoic acid (Nicousamide); and salts and any combinations thereof.

In some embodiments, the ACE2 modulator is selected from the group consisting of a compound of any one of Table 1, Table 2, Table 3, Table 4, Table 5, Table 6, Table 7 and Table 8.

In some embodiments, ACE2 modulator is administered according to a dose regimen selected from the group consisting of once daily (q.d.), twice daily (b.i.d.) thrice daily (t.i.d.), once a week, twice a week, three times a week, once every 2 weeks, once every three weeks, or once a month.

In some embodiments, the ACE2 modulator is administered in a pharmaceutical composition, wherein the composition further comprises at least one pharmaceutically acceptable excipient.

In some embodiments, the ACE2 modulator is administered in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream.

In some embodiments, the pharmaceutical composition is formulated for oral, topical, mucosal, intranasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural, sublingual oral, intranasal, intravenous, intraarterial, intrathecal, vaginal, rectal or subcutaneous administration.

In some embodiments, the present disclosure relates to a pharmaceutical composition in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream, the composition comprising an ACE2 modulator and at least one pharmaceutically acceptable excipient, wherein the ACE2 modulator is selected from the group consisting (S,S)-2-(1-Carboxy-2-(3-(3,5-dichlorobenzyl)-3H-imidazol-4-yl)-ethylamino)-4-methylpentanoic acid (MLN-4760); (2S)-2-acetamido-3-phenylpropanoic acid, or N-Acetyl-DL-phenylalanine (MR708); (2S)-2-[[(2S,3R)-3-amino-2-hydroxy-4-phenylbutanoyl]amino]-4-methylpentanoic acid (Ubenimex); (S)-2-amino-3-(4-boronophenyl)propanoic acid (Borofalan (10B); (2-phenylacetyl)-L-glutamine (Antineoplaston AS2-5); 124-I-4-iodo-phenylalanine (124-I-TLX-101); 131-I-4-iodo-phenylalanine (131-I-TLX-101); ethyl (2S)-2-[[2-(acetylsulfanylmethyl)-3-(2-methylphenyl)propanoyl]amino]-4-methylsulfanylbutanoate (SCH-42495); (((S)-1-carboxy-5((4-iodobenzyl)amino) pentyl)carbamoyl)-L-glutamic acid (Iofolastat-I 123); 8-guanidino-octanoyl-Asp-Phe (SC-49992); (S)-5-amino-2-((1-propyl-1H-imidazol-4-yl)methyl)pentanoic acid (UK-369082); 4-[[2-(1H-imidazol-4-yl)ethyl]carbamoyl]butanoic acid (Ingavirin®); 4-hydroxy-N,1-dimethyl-2-oxo-N-phenyl-3-quinolinecarboxamide (Roquinimex); 5-chloro-N-ethyl-4-hydroxy-1-methyl-2-oxo-N-phenyl-1,2-dihydroquinoline-3-carboxamide (Laquinimod); 4-hydroxy-5-methoxy-N,1-dimethyl-2-oxo-N-[4-(trifluoromethyl)phenyl]quinoline-3-carboxamide (Tasquinimod); Acetyl-L-leucine; (−)-N-[(trans-4-isopropylcyclohexane)carbonyl]-D-phenylalanine (Nateglinide); S-(((R)-2-acetamido-2-carboxyethyl)thio)-N-acetyl-D-cysteine (N,N′-diacetyl-L-cystine); 2-Hydroxy-5-[(7-hydroxy-8-methyl-6-nitro-2-oxochromene-3-carbonyl)amino]benzoic acid (Nicousamide); and salts and any combinations thereof and salts and combinations thereof.

In some embodiments, the present invention relates to a pharmaceutical composition in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream, the composition comprising an ACE2 modulator and at least one pharmaceutically acceptable excipient, wherein the ACE2 modulator is selected from the group consisting of a compound of any one of Table 1, Table 2, Table 3, Table 4, Table 5, Table 6, Table 7 or Table 8.

Further embodiments and the full scope of applicability of the present disclosure will become apparent from the detailed description given hereinafter. However, it should be understood that the detailed description and specific examples, while indicating preferred embodiments of the disclosure, are given by way of illustration only, since various changes and modifications within the spirit and scope of the present disclosure will become apparent to those skilled in the art from this detailed description

### DETAILED DESCRIPTION

It will be appreciated and understood that the present compounds and compositions evaluated and useful for treatment of Covid-19 and other Covid or respiratory illness or the symptoms of Covid-19 and other Covid or respiratory illness were identified, in one implementation, based on an analysis using bioinformatic systems developed by Accencio LLC of Philadelphia. Such bioinformatic systems include those described in U.S. Pat. Nos. 10,013,467 and 10,372,713 which are herein incorporated by reference as if presented in their respective entireties. By way of example, the disclosed compounds used to treat the symptoms of Covid-19 and other related illnesses are obtained using a processor configured to carry out a series of steps in order to create, maintain and manage associations between source documents, the representational identifiers found within the source documents, and any converted coded forms of the representational identifiers. In one implementation, the processor is configured to evaluate existing compounds or compositions that have utility in treating similar viral infections or symptom generators (such as cytokine storms) and generate or identify, based on a n-dimensional landscape mapping of coded forms of the compounds or compositions, those compounds or compositions that are computationally determined to have similar efficiency based on structural or functional similarities based on positioning within a virtual manifold. Those compounds meeting a given threshold of similarity are identified and provided for herein.

As used herein and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state and remission (whether partial or total), whether detectable or undetectable. “Treatment” can also mean prolonging survival as compared to expected survival if not receiving treatment.

As used herein and as well understood in the art, the term an “effective amount,” “sufficient amount” or “therapeutically effective amount” of an agent as used herein interchangeably, is that amount sufficient to effectuate beneficial or desired results, including preclinical and/or clinical results and, as such, an “effective amount” or its variants depends upon the context in which it is being applied. The response is in some embodiments preventative, in others therapeutic, and in others a combination thereof. The term “effective amount” also includes the amount of a compound of the disclosure, which is “therapeutically effective” and which avoids or substantially attenuates undesirable side effects.

As used herein and as well known in the art, and unless otherwise defined, the term “subject” means an animal, including but not limited a human, monkey, cow, horse, sheep, pig, chicken, turkey, quail, cat, dog, mouse, rat, rabbit, or guinea pig. In one embodiment, the subject is a mammal and in another embodiment the subject is a human coronavirus patient.

### Angiotensin Converting Enzyme-2 (ACE2) Modulators

Angiotensin Converting Enzyme-2 (ACE2) is a membrane receptor expressed in a variety of human organs including brain, heart, oral and nasal mucosa, kidney, nasopharynx, colon, lymph nodes, small intestine, stomach, thymus, skin spleen bone marrow, liver and blood vessels. ACE2 expression is high in the lung alveolar epithelial cells, which accounts for most of the damage to the lungs resulting from acute lung damage, ARDS and pneumonia. The coronaviruses SARS-CoV and SARS-CoV-19 enter their host cell through the ACE2 receptor. The glycoprotein spikes on the surface of SARS-CoV and SARS-Cov-19 utilize membrane ACE2 receptors in order to enter the target cells. Thus, ACE2 acts as a transmembrane enzyme with extracellular domain and provides a target site for the virus to mediate its actions in the body. It has now been discovered that targeting ACE2 provides a novel therapeutic target for combating SARS-CoV-19 infections.

In some embodiments, a compound for use in the methods of the present disclosure is an ACE2 modulator. In other embodiments, the compound is an ACE2 inhibitor. An ACE2 inhibitor is characterized by the ability to inhibit the ACE2 receptor with an IC50 of 25 μM or less. By way of illustration, an ACE2 inhibitor is characterized by the ability to inhibit the ACE2 receptor with an IC50 of about 25 μM, 15 μM, 10 μM, 7.5 μM, 5 μM, 2.5 μM, 1.5 μM, 1 μM, 0.5 μM, 0.25 μM, 0.1 μM, 0.01 μM, or about 0.001 μM, or about 0.0001 μM.

In some embodiments, the ACE2 modulator is PV-1001, also known as MLN-4760. N-4760 is a human ACE2 inhibitor (IC₅₀, 0.44 nM). MLN-4760 is chemically designated (S,S)-2-(1-carboxy-2-(3-(3,5-dichlorobenzyl)-3H-imidazol-4-yl)-ethylamino)-4-methylpentanoic acid, and its structure is shown below:

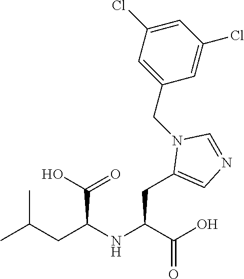 [SMILES: CC(C)C[C@H](N[C@@H](Cc1cncn1Cc1cc(Cl)cc(Cl)c1)C(=O)O)C(=O)O]

MLN-4760 and structurally related compounds are described in PCT International Patent Application WO 00/66104, the contents of which are hereby incorporated by reference for all purposes and the specific purposes identified herein. In some embodiments, such compounds are represented by any one or more of the structures shown in Table 1. Any one of the compounds depicted in Table 1 is suitable for use in the methods of the present disclosure.

TABLE 1
Compound

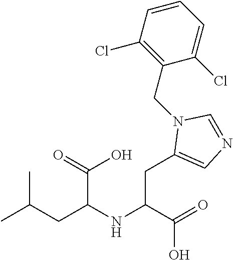 [SMILES: CC(C)CC(NC(Cc1cncn1Cc1c(Cl)cccc1Cl)C(=O)O)C(=O)O]

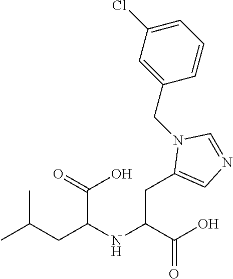 [SMILES: CC(C)CC(NC(Cc1cncn1Cc1cccc(Cl)c1)C(=O)O)C(=O)O]

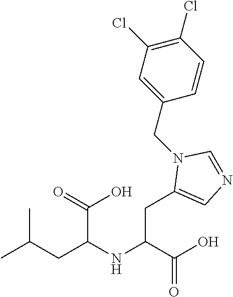 [SMILES: CC(C)CC(NC(Cc1cncn1Cc1ccc(Cl)c(Cl)c1)C(=O)O)C(=O)O]

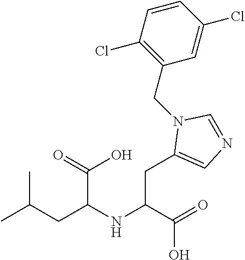 [SMILES: CC(C)CC(NC(Cc1cncn1Cc1cc(Cl)ccc1Cl)C(=O)O)C(=O)O]

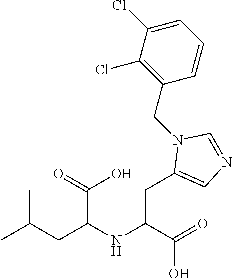 [SMILES: CC(C)CC(NC(Cc1cncn1Cc1cccc(Cl)c1Cl)C(=O)O)C(=O)O]

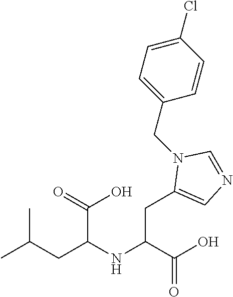 [SMILES: CC(C)CC(NC(Cc1cncn1Cc1ccc(Cl)cc1)C(=O)O)C(=O)O]

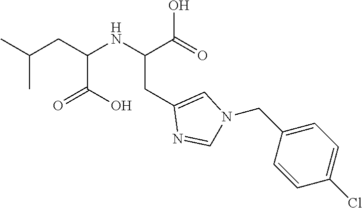 [SMILES: CC(C)CC(NC(Cc1cn(Cc2ccc(Cl)cc2)cn1)C(=O)O)C(=O)O]

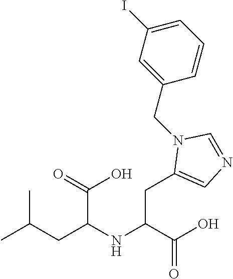 [SMILES: CC(C)CC(NC(Cc1cncn1Cc1cccc(I)c1)C(=O)O)C(=O)O]

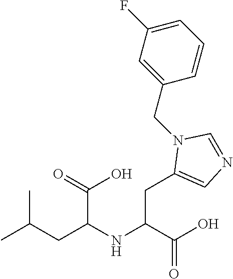 [SMILES: CC(C)CC(NC(Cc1cncn1Cc1cccc(F)c1)C(=O)O)C(=O)O]

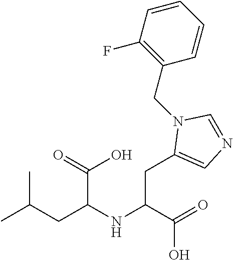 [SMILES: CC(C)CC(NC(Cc1cncn1Cc1ccccc1F)C(=O)O)C(=O)O]

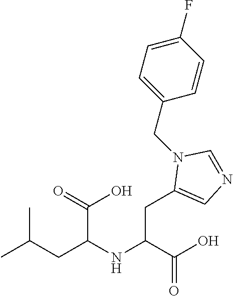 [SMILES: CC(C)CC(NC(Cc1cncn1Cc1ccc(F)cc1)C(=O)O)C(=O)O]

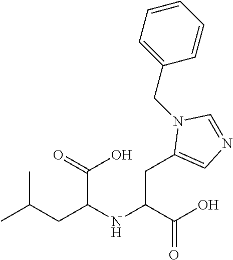 [SMILES: CC(C)CC(NC(Cc1cncn1Cc1ccccc1)C(=O)O)C(=O)O]

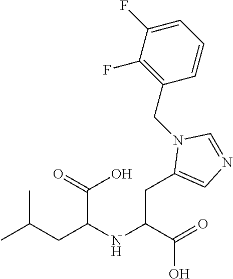 [SMILES: CC(C)CC(NC(Cc1cncn1Cc1cccc(F)c1F)C(=O)O)C(=O)O]

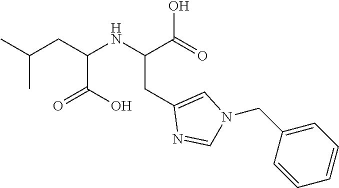 [SMILES: CC(C)CC(NC(Cc1cn(Cc2ccccc2)cn1)C(=O)O)C(=O)O]

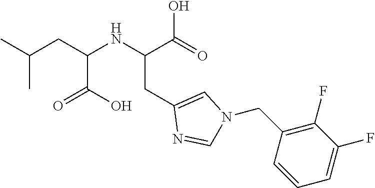 [SMILES: CC(C)CC(NC(Cc1cn(Cc2cccc(F)c2F)cn1)C(=O)O)C(=O)O]

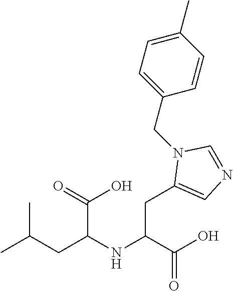 [SMILES: Cc1ccc(Cn2cncc2CC(NC(CC(C)C)C(=O)O)C(=O)O)cc1]

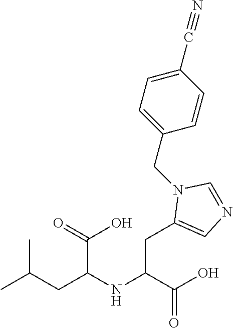 [SMILES: CC(C)CC(NC(Cc1cncn1Cc1ccc(C#N)cc1)C(=O)O)C(=O)O]

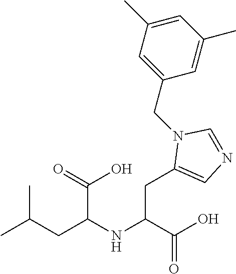 [SMILES: Cc1cc(C)cc(Cn2cncc2CC(NC(CC(C)C)C(=O)O)C(=O)O)c1]

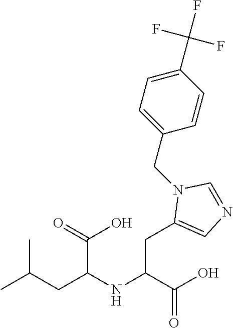 [SMILES: CC(C)CC(NC(Cc1cncn1Cc1ccc(C(F)(F)F)cc1)C(=O)O)C(=O)O]

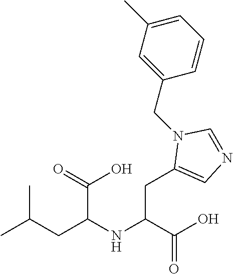 [SMILES: Cc1cccc(Cn2cncc2CC(NC(CC(C)C)C(=O)O)C(=O)O)c1]

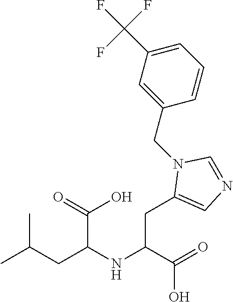 [SMILES: CC(C)CC(NC(Cc1cncn1Cc1cccc(C(F)(F)F)c1)C(=O)O)C(=O)O]

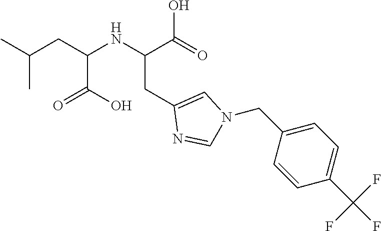 [SMILES: CC(C)CC(NC(Cc1cn(Cc2ccc(C(F)(F)F)cc2)cn1)C(=O)O)C(=O)O]

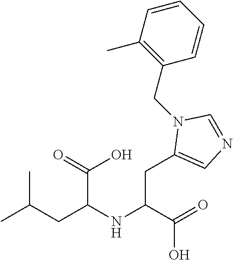 [SMILES: Cc1ccccc1Cn1cncc1CC(NC(CC(C)C)C(=O)O)C(=O)O]

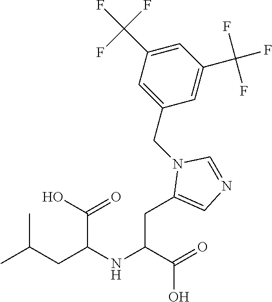 [SMILES: CC(C)CC(NC(Cc1cncn1Cc1cc(C(F)(F)F)cc(C(F)(F)F)c1)C(=O)O)C(=O)O]

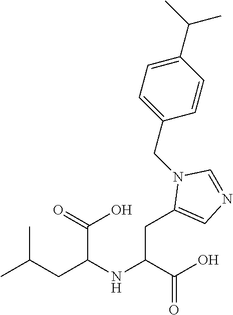 [SMILES: CC(C)CC(NC(Cc1cncn1Cc1ccc(C(C)C)cc1)C(=O)O)C(=O)O]

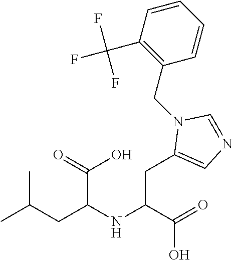 [SMILES: CC(C)CC(NC(Cc1cncn1Cc1ccccc1C(F)(F)F)C(=O)O)C(=O)O]

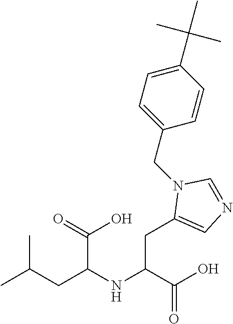 [SMILES: CC(C)CC(NC(Cc1cncn1Cc1ccc(C(C)(C)C)cc1)C(=O)O)C(=O)O]

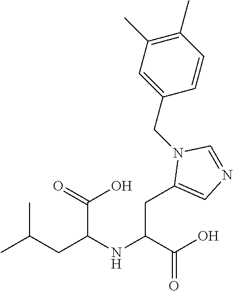 [SMILES: Cc1ccc(Cn2cncc2CC(NC(CC(C)C)C(=O)O)C(=O)O)cc1C]

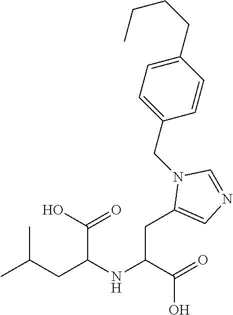 [SMILES: CCCCc1ccc(Cn2cncc2CC(NC(CC(C)C)C(=O)O)C(=O)O)cc1]

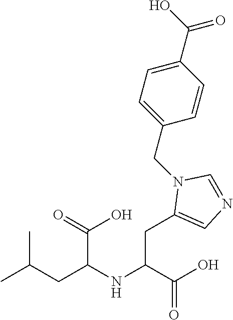 [SMILES: CC(C)CC(NC(Cc1cncn1Cc1ccc(C(=O)O)cc1)C(=O)O)C(=O)O]

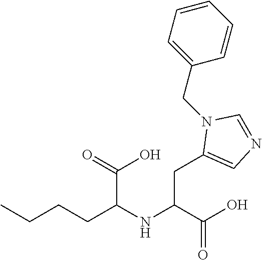 [SMILES: CCCCC(NC(Cc1cncn1Cc1ccccc1)C(=O)O)C(=O)O]

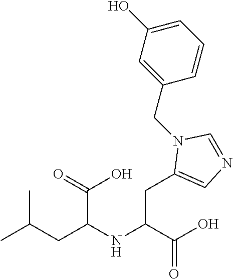 [SMILES: CC(C)CC(NC(Cc1cncn1Cc1cccc(O)c1)C(=O)O)C(=O)O]

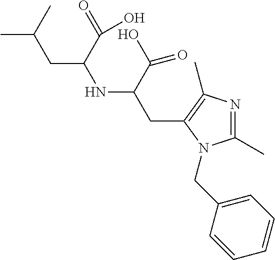 [SMILES: Cc1nc(C)n(Cc2ccccc2)c1CC(NC(CC(C)C)C(=O)O)C(=O)O]

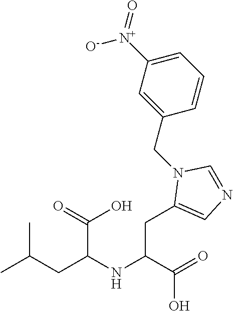 [SMILES: CC(C)CC(NC(Cc1cncn1Cc1cccc([N+](=O)[O-])c1)C(=O)O)C(=O)O]

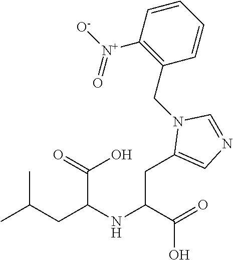 [SMILES: CC(C)CC(NC(Cc1cncn1Cc1ccccc1[N+](=O)[O-])C(=O)O)C(=O)O]

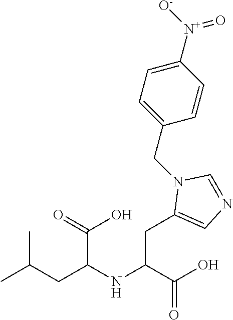 [SMILES: CC(C)CC(NC(Cc1cncn1Cc1ccc([N+](=O)[O-])cc1)C(=O)O)C(=O)O]

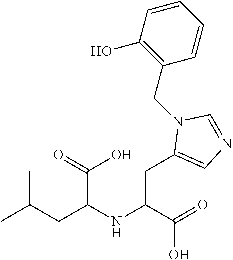 [SMILES: CC(C)CC(NC(Cc1cncn1Cc1ccccc1O)C(=O)O)C(=O)O]

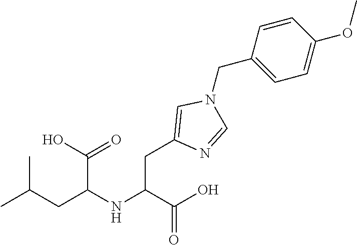 [SMILES: COc1ccc(Cn2cnc(CC(NC(CC(C)C)C(=O)O)C(=O)O)c2)cc1]

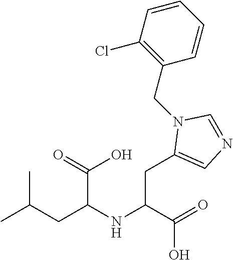 [SMILES: CC(C)CC(NC(Cc1cncn1Cc1ccccc1Cl)C(=O)O)C(=O)O]

In some embodiments, the compound is MR708, which is chemically designated (2S)-2-acetamido-3-phenylpropanoic acid, or N-Acetyl-DL-phenylalanine. The structure of MR708 is represented below:

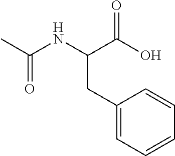

In other embodiments, the compound is Ubenimex, also known as bestatin, which is chemically designated (2S)-2-[[(2S,3R)-3-amino-2-hydroxy-4-phenylbutanoyl]amino]4 methylpentanoic acid. The structure of Ubenimex is represented below. Ubenimex is an inhibitor of arginyl aminopeptidase, leukotriene A4 hydrolase, alanyl aminopeptidase, leucyl/cystinyl aminopeptidase, and membrane dipeptidase. The compound was in clinical development for the treatment of Pulmonary Arterial Hypertension (PAH), Lymphedema and cancer. In some embodiments, Ubenimex is administered orally. In other embodiments, Ubenimex is administered orally as a capsule, at a dose of 150 mg thrice daily.

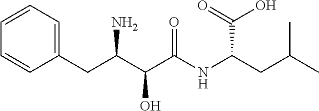 [SMILES: CC(C)C[C@H](NC(=O)[C@@H](O)[C@H](N)Cc1ccccc1)C(=O)O]

In some embodiments, the compound is Borofalan (10B), also known as SPM-11. Borofalan (10B) is developed for the treatment of cancer. The chemical structure of Borofalan (10B) is (S)-2-amino-3-(4-boronophenyl)propanoic acid, and its structure is shown below.

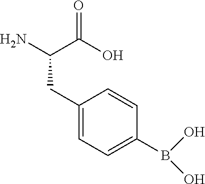 [SMILES: N[C@@H](Cc1ccc(B(O)O)cc1)C(=O)O]

In some embodiments, the compound is Antineoplaston AS2-5, a glutamate receptor modulator previously in clinical trials for the treatment of glioma. Aritireoplaston AS2-5 is chemically designated (2-phenylacetyl)-L-glutamine, and its structure is shown below.

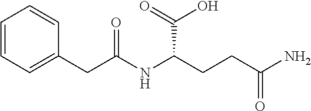 [SMILES: NC(=O)CC[C@H](NC(=O)Cc1ccccc1)C(=O)O]

In some embodiments, the compound is 124-I-TLX-101 or 131-TLX-101, iodine-labeled phenylalanine (4-iodo-phenylalanine) derivatives in phase clinical trials for cancer. The structures of these compounds are provided below.

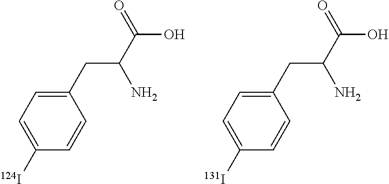 [SMILES: NC(Cc1ccc([124I])cc1)C(=O)O ; NC(Cc1ccc([131I])cc1)C(=O)O]

In some embodiments, the compound is SCH-42495, an endopeptidase inhibitor chemically named ethyl (2S)-2-[[2-(acetylsulfanylmethyl)-3-(2-methylphenyl)propanoyl]amino]-4-methylsulfanylbutanoate. The structure of SCH-42495 is provided below:

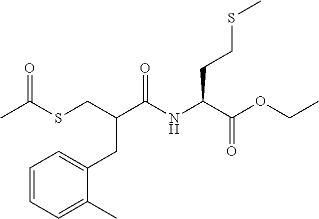 [SMILES: CCOC(=O)[C@H](CCSC)NC(=O)C(CSC(C)=O)Cc1ccccc1C]

In some embodiments, the compound is Iofolastat-I 123, a glutamate carboxypeptidase II inhibitor which is chemically designated (((S)-1-carboxy-5-((4-iodobenzyl)amino)pentyl)carbamoyl)-L-glutamic acid. The structure of Iofolastat-I124 is provided below:

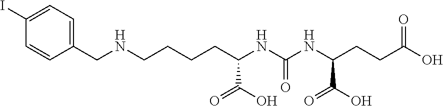 [SMILES: O=C(O)CC[C@H](NC(=O)N[C@@H](CCCCNCc1ccc(I)cc1)C(=O)O)C(=O)O]

In some embodiments, the compound is SC-49992, an inhibitor of platelet aggregation. SC-49992 inhibits the binding of fibrinogen to its receptor on activated platelets, glycoprotein IIb/IIIa, wherein fibrinogen binding is required for platelet aggregation and subsequent thrombus formation. SC-49992 is chemically designated 8-guanidino-octanoyl-Asp-Phe, and its structure is shown below.

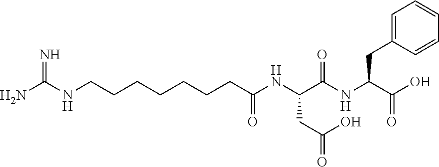 [SMILES: N=C(N)NCCCCCCCC(=O)N[C@@H](CC(=O)O)C(=O)N[C@@H](Cc1ccccc1)C(=O)O]

Structurally related compounds to MR708, Ubenimex, Borofalan, antineoplaston AS2-5, 124-I-TLX-101, 131-I-TLX-101, SCH-42495, lofolastat-I 123 and SC-49992 are described in PCT International Patent Application WO 00/66104, the contents of which are hereby incorporated by reference for all purposes and the specific purposes identified herein. In some embodiments, such compounds are represented by any one or more of the structures shown in Table 2. Any one of the compounds depicted in Table 2 is suitable for use in the methods of the present disclosure.

TABLE 2
Compound

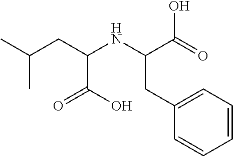 [SMILES: CC(C)CC(NC(Cc1ccccc1)C(=O)O)C(=O)O]

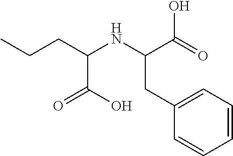 [SMILES: CCCC(NC(Cc1ccccc1)C(=O)O)C(=O)O]

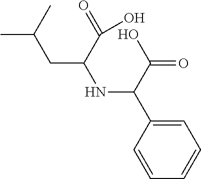 [SMILES: CC(C)CC(NC(C(=O)O)c1ccccc1)C(=O)O]

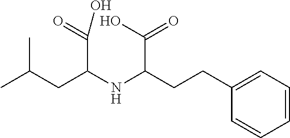 [SMILES: CC(C)CC(NC(CCc1ccccc1)C(=O)O)C(=O)O]

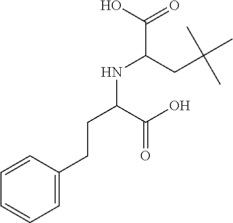 [SMILES: CC(C)(C)CC(NC(CCc1ccccc1)C(=O)O)C(=O)O]

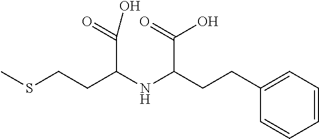 [SMILES: CSCCC(NC(CCc1ccccc1)C(=O)O)C(=O)O]

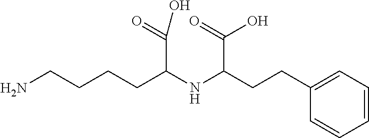 [SMILES: NCCCCC(NC(CCc1ccccc1)C(=O)O)C(=O)O]

In some embodiments, the compound is UK-369082, a thrombin-active fibrinolysis inhibitor previously in clinical development for thrombosis. UK-369082 is chemically designated (S)-5-amino-2-((1-propyl-1H-imidazol-4-yl)methyl)pentanoic acid, and its chemical structure is shown below.

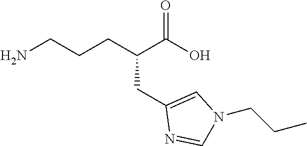 [SMILES: CCCn1cnc(C[C@H](CCCN)C(=O)O)c1]

In some embodiments, the compound is imidazolyl ethanamide pentadioic acid, also known as Ingavirin®. Ingavirin is studied in the clinic for the treatment of influenza and the common cold. Ingavirin® is chemically, designated 4-{[2-(1H-imidazol-4-yl)ethyl]carbamoyl}butanoic acid. The chemical structure of Ingavirin® is shown below.

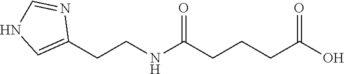 [SMILES: O=C(O)CCCC(=O)NCCc1c[nH]cn1]

Structurally related compounds to UK-369082 and Ingavirin® are described in PCT International Patent Application WO 00/66104, the contents of which are hereby incorporated by reference for all purposes and the specific purposes identified herein. In some embodiments, such compounds are represented by any one or more of the structures shown in Table 3. Any one of the compounds depicted in Table 3 is suitable for use in the methods of the present disclosure.

TABLE 3
Compound

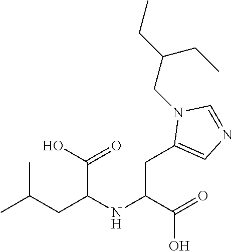 [SMILES: CCC(CC)Cn1cncc1CC(NC(CC(C)C)C(=O)O)C(=O)O]

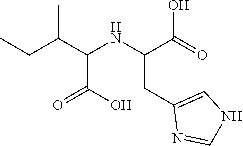 [SMILES: CCC(C)C(NC(Cc1c[nH]cn1)C(=O)O)C(=O)O]

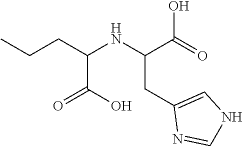 [SMILES: CCCC(NC(Cc1c[nH]cn1)C(=O)O)C(=O)O]

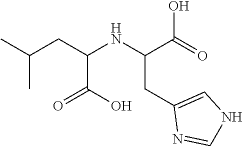 [SMILES: CC(C)CC(NC(Cc1c[nH]cn1)C(=O)O)C(=O)O]

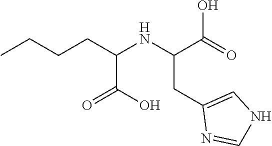 [SMILES: CCCCC(NC(Cc1c[nH]cn1)C(=O)O)C(=O)O]

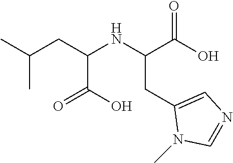 [SMILES: CC(C)CC(NC(Cc1cncn1C)C(=O)O)C(=O)O]

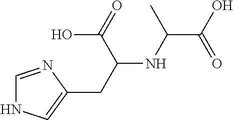 [SMILES: CC(NC(Cc1c[nH]cn1)C(=O)O)C(=O)O]

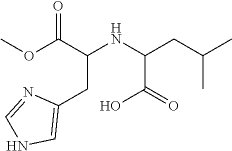 [SMILES: COC(=O)C(Cc1c[nH]cn1)NC(CC(C)C)C(=O)O]

In some embodiments, the compound is Roquinimex (Linomide). Roquinimex is a quinoline derivative immunostimulant which increases NK cell activity and macrophage cytotoxicity, inhibits angiogenesis and reduces the secretion of TNF alpha. Roquinimex has been investigated as a treatment of cancers and autoimmune diseases, such as multiple sclerosis and recent-onset type I diabetes. Roquinimex is chemically designated 4-hydroxy-N,1-dimethyl-2-oxo-N-phenyl-3-quinolinecarboxamide, and its structure is shown below.

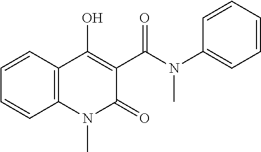 [SMILES: CN(C(=O)c1c(O)c2ccccc2n(C)c1=O)c1ccccc1]

In some embodiments, the compound is Laquinimod, which was developed as a successor to Linomide for the treatment of multiple sclerosis. The chemical name of Laquinimod is 5-chloro-N-ethyl-4-hydroxy-1-methyl-2-oxo-N-phenyl-1,2-dihydroquinoline-3-carboxamide, and its structure is shown below.

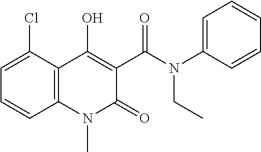 [SMILES: CCN(C(=O)c1c(O)c2c(Cl)cccc2n(C)c1=O)c1ccccc1]

In some embodiments, the compound is Tasquinimod, an S100 calcium binding protein A9 modulator. Tasquinimod is in clinical trials for the treatment of cancer. The chemical name of Tasquinimod is 4-hydroxy-5-methoxy-N,1-dimethyl-2-oxo-N-[4-(trifluoromethyl)phenyl]quinoline-3-carboxamide, and its structure is shown below. In one embodiment, Tasquinimod is administered orally. In another embodiment, Tasquinimod is administered orally in a capsule at doses ranging from about 0.1 mg/kg to about 1 mg/kg.

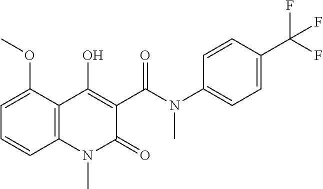 [SMILES: COc1cccc2c1c(O)c(C(=O)N(C)c1ccc(C(F)(F)F)cc1)c(=O)n2C]

Structurally related compounds to Roquirtimex, Laquinimod and Tasquinimod are described in PCT International Patent Application WO 2007/124617, the contents of which are hereby incorporated by reference for all purposes and the specific purposes identified herein. In some embodiments, such compounds are represented by any one or more of the structures shown in Table 4. Any one of the compounds depicted in Table 4 is suitable for use in the methods of the present disclosure.

TABLE 4
Compound

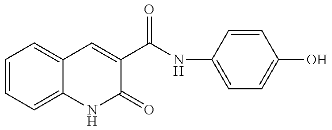 [SMILES: O=C(Nc1ccc(O)cc1)c1cc2ccccc2[nH]c1=O]

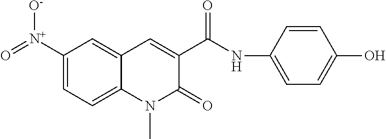 [SMILES: Cn1c(=O)c(C(=O)Nc2ccc(O)cc2)cc2cc([N+](=O)[O-])ccc21]

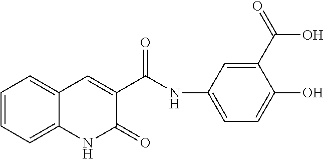 [SMILES: O=C(O)c1cc(NC(=O)c2cc3ccccc3[nH]c2=O)ccc1O]

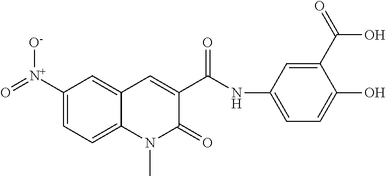 [SMILES: Cn1c(=O)c(C(=O)Nc2ccc(O)c(C(=O)O)c2)cc2cc([N+](=O)[O-])ccc21]

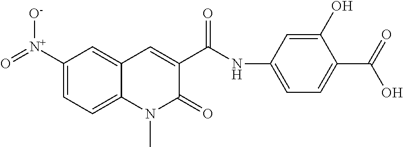 [SMILES: Cn1c(=O)c(C(=O)Nc2ccc(C(=O)O)c(O)c2)cc2cc([N+](=O)[O-])ccc21]

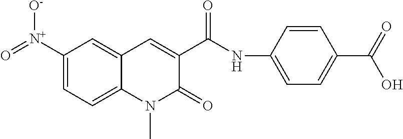 [SMILES: Cn1c(=O)c(C(=O)Nc2ccc(C(=O)O)cc2)cc2cc([N+](=O)[O-])ccc21]

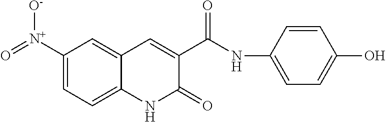 [SMILES: O=C(Nc1ccc(O)cc1)c1cc2cc([N+](=O)[O-])ccc2[nH]c1=O]

In some embodiments, the compound is acetyl-L-leucine, the structure of which is shown below. Acetyl-L-leucine is used for the treatment of cerebellar ataxia, Niemann-Pick disease and Tay-Sachs disease.

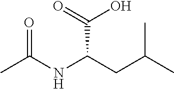

Structurally related compounds to acetyl-L-leucine are described in PCT International Patent Application WO 00/66104, the contents of which are hereby incorporated by reference for all purposes and the specific purposes identified herein. In some embodiments, such compounds are represented by any one or more of the structures shown in Table 5. Any one of the compounds depicted in Table 5 is suitable for use in the methods of the present disclosure.

TABLE 5
Compound

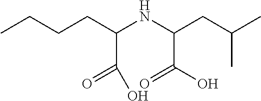 [SMILES: CCCCC(NC(CC(C)C)C(=O)O)C(=O)O]

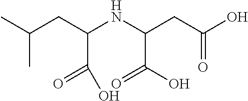 [SMILES: CC(C)CC(NC(CC(=O)O)C(=O)O)C(=O)O]

In some embodiments, the compound is Nateglinide (STARLIX ®), a compound used to lower blood sugar in type 2 diabetes. The chemical name of Nateglinide is (−)-N-[(trans-4-isopropylcyclohexane)carbonyl]-D-phenylalanine, and its structure is shown below. In some embodiments, Nateglinide is administered orally. In other embodiments, Nateglinide is administered orally as a tablet.

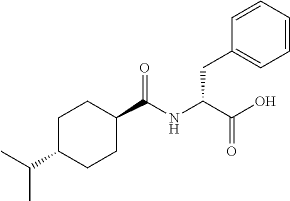 [SMILES: CC(C)[C@H]1CC[C@H](C(=O)N[C@H](Cc2ccccc2)C(=O)O)CC1]

Structurally related compounds to Nateglinide are described in PCT International Patent Application WO 00/66104, the contents of which are hereby incorporated by reference for all purposes and the specific purposes identified herein. In some embodiments, a compound of Table 6 is suitable for use in the methods of the present disclosure.

TABLE 6
Compound

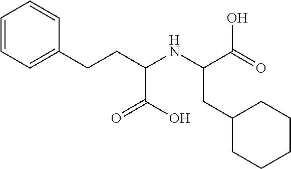 [SMILES: O=C(O)C(CCc1ccccc1)NC(CC1CCCCC1)C(=O)O]

In some embodiments, the compound is N,N′-diacetyl-L-cystine (DiNAC), which is chemically designated S-(((R)-2-acetamido-2-carboxyethyl)thio)-N acetyl-D-cysteine. N,N′-diacetyl-L-cystine is a disulfide dimer of N-acetylcysteine with immunomodulatory properties. The structure of N,N-diacetyl-L-cystine is shown below.

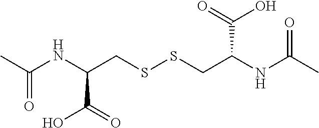 [SMILES: CC(=O)N[C@@H](CSSC[C@@H](NC(C)=O)C(=O)O)C(=O)O]

Structurally related compounds to N,N′-diacetyl-L-cystine are described in PCT International Patent Application WO 00/66104, the contents of which are hereby incorporated by reference for all purposes and the specific purposes identified herein. In some embodiments, such compounds are represented by the compound shown in Table 7.

TABLE 7
Compound

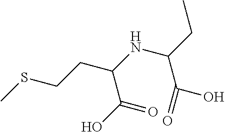

In some embodiments, the compound is Nicousamide, which is chemically designated 2-Hydroxy-5-[(7-hydroxy-8-methyl-6-nitro-2-oxochromene-3-carbonyl)amino]benzoic acid. Nicousamide is a renin and TGF-beta activated kinas-1 inhibitor which is in clinical development for renal disease. The structure of Nicousamide is shown below.

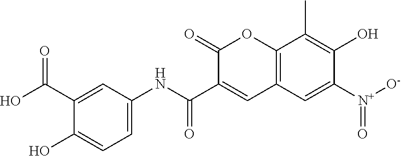 [SMILES: Cc1c(O)c([N+](=O)[O-])cc2cc(C(=O)Nc3ccc(O)c(C(=O)O)c3)c(=O)oc12]

Structurally related compounds to Nicousamide are described in PCT International Patent Application WO 2007/124617, the contents of which are hereby incorporated by reference for all purposes and the specific purposes identified herein. in some embodiments, such compounds are represented by the compound shown in Table 8.

TABLE 8
Compound

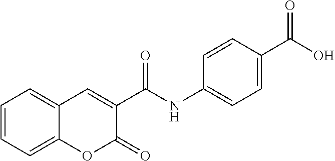 [SMILES: O=C(O)c1ccc(NC(=O)c2cc3ccccc3oc2=O)cc1]

### Methods of Treatment

In certain embodiments, the compositions and methods of the present disclosure are useful for the prevention and/or treatment of symptoms of SARS-CoV-19 infections. In certain embodiments, the compositions and methods of the present disclosure are useful for 15 the prevention and/or treatment of acute inflammatory responses. In certain embodiments, the compositions and methods of the present disclosure are useful for the prevention and/or treatment of acute inflammatory responses, e.g., cytokine storms that are associates with a coronavirus infection.

The present disclosure is based on the discovery that angiotensin converting enzyme-2 (ACE2) modulators may have therapeutic utility in the treatment of coronavirus symptoms, in particular in preventing cytokine storms in critical patients with coronavirus infections, in particular SARS-COV-19.

Thus, in some embodiments, ACE2 modulators (e.g., ACE2 inhibitors) may prevent onset of severe SARS-CoV-19 symptoms. For example, ACE2 modulators (e.g., ACE2 inhibitors) may prevent or ameliorate the hyper-inflammatory response in patients with SARS-CoV-19 pneumonia and prevent or ameliorate progress to cytokine storm. Successful intervention with an ACE2 modulator (e.g., an ACE2 inhibitor) may reduce life-threatening complications of SARS-COV-19, including severe respiratory symptoms that often necessitate further medical intervention such as mechanical intervention.

Thus, in some embodiments, the present disclosure, relates to a method of treating or alleviating at least one symptom of a coronavirus infection in a subject, by administering to the subject a therapeutically effective amount of an ACE2 modulator. In some embodiments, the subject is a human.

In some embodiments, the symptom is fever. In other embodiments, the symptom is cough. In other embodiments, the symptom is dry cough. In other embodiments, the symptom is tiredness. In other embodiments, the symptom is sore throat. In other embodiments, the symptom is diarrhea. In other embodiments, the symptom is conjunctivitis. In other embodiments, the symptom is headache. In other embodiments, the symptom is loss of taste. In other embodiments, the symptom is loss of smell. In other embodiments, the symptom is a rash. In other embodiments, the symptom is difficulty breathing. In other embodiments, the symptom is shortness of breath. In other embodiments, the symptom is chest pain. In other embodiments, the symptom is chest pressure. In other embodiments, the symptom is Acute Respiratory Distress Syndrome (ARDS). In other embodiments, the symptom is organ failure. In other embodiments, the symptom is multiple organ failure. In other embodiments, the symptom is any combination of the foregoing.

In some embodiments, the present disclosure relates to a method of treating an acute inflammatory condition in a subject infected with a coronavirus, the method comprising the step of administering to the subject a therapeutically effective amount of an ACE2 modulator. In some embodiments, the inflammatory condition comprises a cytokine storm. In some embodiments, the subject is a human.

In some embodiments, the present disclosure relates to a method of preventing a cytokine storm in a subject infected with a coronavirus, the method comprising the step of administering to the subject a therapeutically effective amount of an ACE2 modulator. In some embodiments, the subject is a human.

In some embodiments, the present disclosure relates to a method of reducing or arresting viral load in a subject infected with a coronavirus, the method comprising the step of administering to the subject a therapeutically effective amount of an ACE2 modulator. In some embodiments, the subject is a human.

Viral load can be measured by any viral diagnostic equipment or technique known in the art. A wide variety of samples can be used for virological testing. Such samples include, but are not limited to, upper respiratory swabs (nasopharyngeal swabs, nasopharyngeal wash/aspirate, oropharyngeal swabs, saliva) and lower respiratory specimens (sputum, bronchoalveolar lavage, lung tissue), as well as stool, rectal swabs, blood, skin, urine, semen, faeces, cerebrospinal fluid, tissue (e.g., biopsies), and the like. Techniques for measuring viral load include, but are not limited to, nucleic acid amplification-based tests (NATs) or non-nucleic acid-based tests. Examples of NATs include, but are not limited to, PCR (polymerase chain reaction), reverse transcription polymerase chain reaction (RT-PCR), and nucleic acid sequence-based amplification (NASBA). Viral load is typically reported as copies the virus in a milliliter (mL) of blood. Changes in viral load are usually reported as a log change (in powers of 10). For example, a three-log increase in viral load (3 log10) is an increase of 10³ or LOGO times the previously reported level, while a drop from 500,000 to 500 copies would be a three-log-drop.

In one embodiment, the subject is infected with a coronavirus. In some embodiments, the coronavirus is selected from the group consisting of 229E (alpha coronavirus), NL63 (alpha coronavirus), OC43 (beta coronavirus), HKU1 (beta coronavirus), MERS-CoV (beta coronavirus that causes Middle East Respiratory Syndrome, or MERS), SARS-CoV (the beta coronavirus that causes severe acute respiratory syndrome, or SARS) SARS-CoV-2 (the novel coronavirus that causes coronavirus disease 2019, or COVID-19, also referred to herein as SARS-Covid-19). In some embodiments, the coronavirus is a severe acute respiratory syndrome coronavirus (SARS-CoV). In some embodiments, the coronavirus is a novel virus 2019-nCoV (SARS-CoV-19). In some embodiments, the coronavirus is a Middle East respiratory syndrome coronavirus (MERS-CoV). In one preferred embodiment, the coronavirus is SARS-CoV-19.

### Pharmaceutical Compositions

The present disclosure thus provides pharmaceutical compositions comprising ACE2 modulators and a pharmaceutically acceptable carrier. The compounds of the present disclosure can be formulated as pharmaceutical compositions and administered to a mammalian host, such as a human patient, in a variety of forms adapted to the chosen route of administration.

Routes of administration include, but are not limited to oral, topical, mucosal, nasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural and sublingual administration.

As used herein, the term “administering” generally refers to any and all means of introducing compounds described herein to the host subject. Compounds described herein may be administered in unit dosage forms and/or compositions containing one or more pharmaceutically-acceptable carriers, adjuvants, diluents, excipients, and/or vehicles, and combinations thereof.

As used herein, the terms “composition” generally refers to any product comprising more than one ingredient, including the compounds described herein. It is to be understood that the compositions described herein may be prepared from compounds described herein or from salts, solutions, hydrates, solvates, and other forms of the compounds described herein.

It is appreciated that the compositions may be prepared from various amorphous, non-amorphous, partially crystalline, crystalline, and/or other morphological forms of the compounds described herein, and the compositions may be prepared from various hydrates and/or solvates of the compounds described herein. Accordingly, such pharmaceutical compositions that recite compounds described herein include each of, or any combination of, or individual forms of, the various morphological forms and/or solvate or hydrate forms of the compounds described herein.

In some embodiments, the ACE2 modulator may be systemically (e.g., orally) administered in combination with a pharmaceutically acceptable vehicle such as an inert diluent or an assimilable edible carrier. For oral therapeutic administration, the active compound may be combined with one or more excipients and used in the form of ingestible tablets, buccal tablets, sublingual tablets, troches, capsules, elixirs, suspensions, syrups, wafers, and the like. The percentage of the compositions and preparations may vary and may be between about 1 to about 99% weight of the active ingredient(s) and excipients such as, but not limited to a binder, a filler, a diluent, a disintegrating agent, a lubricant, a surfactant, a sweetening agent; a flavoring agent, a colorant, a buffering agent, anti-oxidants, a preservative, chelating agents (e.g., ethylenediaminetetraacetic acid), and agents for the adjustment of tonicity such as sodium chloride.

Suitable binders include, but are not limited to, polyvinylpyrrolidone, copovidone, hydroxypropyl methylcellulose, starch, and gelatin.

Suitable fillers include, but are not limited to, sugars such as lactose, sucrose, mannitol or sorbitol and derivatives therefore (e.g. amino sugars), ethylcellulose, microcrystalline cellulose, and silicified microcrystalline cellulose.

Suitable diluents include, but are not limited to, dicalcium phosphate dihydrate, sugars, lactose, calcium phosphate, cellulose, kaolin, mannitol, sodium chloride, and dry starch.

Suitable disintegrants include, but are not limited to, pregelatinized starch, crospovidone, crosslinked sodium carboxymethyl cellulose and combinations thereof.

Suitable lubricants include, but are not limited to, sodium stearyl fumarate, stearic acid, polyethylene glycol or stearates, such as magnesium stearate.

Suitable surfactants or emulsifiers include, but are not limited to. polyvinyl alcohol (PVA), polysorbate, polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil.

Suitable flavoring agents and sweeteners include, but are not limited to, sweeteners such as sucralose and synthetic flavor oils and flavoring aromatics, natural oils, extracts from plants. leaves, flowers, and fruits, and combinations thereof. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot.

Suitable colorants include, but are not limited to, alumina (dried aluminum hydroxide), annatto extract, calcium carbonate, canthaxanthin, caramel, β-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide.

Suitable buffering or pH adjusting agent include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide and magnesium hydroxide.

Suitable tonicity enhancing agents include, but are not limited to, ionic and non-ionic agents such as, alkali metal or alkaline earth metal halides, urea, glycerol, sorbitol, mannitol, propylene glycol, and dextrose.

Suitable wetting agents include, but are not limited to, glycerin, cetyl alcohol, and glycerol monostearate.

Suitable preservatives include, but are not limited to, benzalkonium chloride, benzoxonium chloride, thiomersal, phenylmercuric nitrate, phenylmercuric acetate, phenylmercuric borate, methylparaben, propylparaben. chlorobutanol, benzyl alcohol, phenyl alcohol, chlorohexidine, and polyhexamethylene biguanide.

Suitable antioxidants include, but are not limited to, sorbic acid, ascorbic acid, ascorbate, glycine, α-tocopherol, butylated hydroxyanisole (BHA), and butylated hydroxytoluene (BHT).

The ACE2 modulator of the present disclosure may also be administered via infusion or injection (e.g., using needle (including microneedle) injectors and/or needle-free injectors). Solutions of the active composition can be aqueous, optionally mixed with a nontoxic surfactant and/or may contain carriers or excipients such as salts, carbohydrates and buffering agents (preferably at a pH of from 3 to 9), and, for some applications, they may be more suitably formulated as a sterile non-aqueous solution or as a dried form to be used in conjunction with a suitable vehicle such as sterile, pyrogen-free water or phosphate-buffered saline. For example, dispersions can be prepared in glycerol, liquid polyethylene glycols, triacetin, and mixtures thereof and in oils. The preparations may further contain a preservative to prevent the growth of microorganisms.

The pharmaceutical compositions may be formulated for parenteral administration (e.g., subcutaneous, intravenous, intra-arterial, transdermal, intraperitoneal or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Water is a preferred carrier when the pharmaceutical composition is administered intravenously. Saline solutions and aqueous dextrose and glycerol solutions can also be employed as liquid carriers, particularly for injectable solutions. Oils such as petroleum, animal, vegetable, or synthetic oils and soaps such as fatty alkali metal, ammonium, and triethanolamine salts, and suitable detergents may also be used for parenteral administration. Further, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. Suitable preservatives include e.g. sodium benzoate, benzoic acid, and sorbic acid. Suitable antioxidants include e.g. sulfites, ascorbic acid and □-tocopherol.

The preparation of parenteral compounds/compositions under sterile conditions, for example, by lyophilization, may readily be accomplished using standard pharmaceutical techniques well known to those skilled in the art.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. In one embodiment, the compositions are administered by the oral or nasal respiratory route for local or systemic effect. Compositions in pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, orally or nasally, from devices that deliver the formulation in an appropriate manner.

In yet another embodiment, the composition is prepared for topical administration, e.g. as an ointment, a gel, a drop, a patch or a cream. For topical administration to body surfaces using, for example, creams, gels, drops, ointments and the like, the compounds of the present disclosure can be prepared and applied in a physiologically acceptable diluent with or without a pharmaceutical carrier. Adjuvants for topical or gel base forms may include, for example, sodium carboxymethylcellulose, polyacrylates, polyoxyethylene-polyoxypropylene-block polymers, polyethylene glycol, wood wax alcohols, isostearic acid, cetyl alcohol, stearyl alcohol, white petrolatum, polysorbate 60, sorbitan monostearate, glycerin, xanthan gum, water. benzyl alcohol, methylparaben, and propylparaben. Additional additives may be selected from the group consisting of waxes, soaps, sorbitan esters, fatty acids, fatty acid esters, fatty acid oils, borates, cresol, chlorocresol, cellulose, methylcellulose, hydroxypropylcellulose, acacia, and the like. Examples of suitable topical dosage forms may be found in e.g., Tarun Garg, Goutam Rath & Amit K. Goyal (2015) Comprehensive review on additives of topical dosage forms for drug delivery, Drug Delivery, 22:8, 969-987, the contents of which are hereby incorporated by reference in their entirety.

Alternative formulations include nasal sprays, liposomal formulations, slow-release formulations, pumps delivering the drugs into the body (including mechanical or osmotic pumps) controlled-release formulations and the like, as are known in the art.

### Doses

As used herein, the term “therapeutically effective dose” means (unless specifically stated otherwise) a quantity of a compound which, when administered either one time or over the course of a treatment cycle affects the health, wellbeing or mortality of a subject (e.g., delays the onset of and/or reduces the severity of one or more of the symptoms associated with a coronavirus, e.g., SARS-Covid-19.

An ACE2 modulator described herein can be present in a composition in an amount of about 0.001 mg, about 0.005 mg, about 0.01 mg, about 0.02 mg, about 0.03 mg, about 0.04 mg, about 0.05 mg, about 0.06 mg, about 0.07 mg, about 0.08 mg, about 0.09 mg about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 0.5 mg, about 10 mg, about 10.5 mg, about 11 mg, about 12 mg, about 12.5 mg, about 13 mg, about 13.5 mg, about 14 mg, about 14.5g, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about mg, about 90 mg, about 95 mg, about 100 mg.

An ACE2 modulator described herein described herein can be present in a composition in a range of from about 0.1 mg to about 100 mg; 0.1 mg to about 75 mg; from about 0.1 mg to about 50 mg; from about 0.1 mg to about 25 mg; from about 0.1 mg to about 10 mg; 0.1 mg to about 7.5 mg, 0.1 mg to about 5 mg; 0.1 mg to about 2.5 mg; from about 0.1 mg to about 1 mg; from about 0.5 mg to about 100 mg; from about 0.5 mg to about 75 mg; from about 0.5 mg to about 50 mg; from about 0.5 mg to about 25 mg; from about 0.5 mg to about 10 mg; from about 0.5mg to about 5 mg, from about 0.5mg to about 2.5 mg; from about 0.5 mg to about 1 mg; from about 1 mg to about 100 mg; from about 1 mg to about 75 mg; from about 0.1 mg to about 50 mg; from about 0.1 mg to about 25 mg; from about 0.1 mg to about 10 mg; from about 0.1 mg to about 5 mg; from about 0.1 mg to about 2.5 mg; from about 0.1 mg to about 1 mg.

### Dosing Regimens

The compounds described herein can be administered by any dosing schedule or dosing regimen as applicable to the patient and/or the condition being treated. Administration can be once a day (q.d.), twice a day (b.i.d.), thrice a day (t.i.d.), once a week, twice a week, three times a week, once every 2 weeks, once every three weeks, or once a month twice, and the like.

In some embodiments, the ACE2 modulator is administered for a period of at least one day. In other embodiments, the ACE2 modulator is administered for a period of at least 2 days. In other embodiments, the ACE2 modulator is administered for a period of at least 3 days. In other embodiments, the ACE2 modulator is administered for a period of at least 4 days. In other embodiments, the ACE2 modulator is administered for a period of at least 5 days. In other embodiments, the ACE2 modulator is administered for a period of at least 6 days. In other embodiments, the ACE2 modulator is administered for a period of at least 7 days. In other embodiments, the ACE2 modulator is administered for a period of at least 10 days. In other embodiments, the ACE2 modulator is administered for a period of at least 14 days. In other embodiments, the ACE2 modulator is administered for a period of at least one month. In some embodiments, the ACE2 modulator is administered chronically for as long as the treatment is needed.

The present subject matter described herein will be illustrated more specifically by the following non-limiting examples, it being understood that changes and variations can be made therein without deviating from the scope and the spirit of the disclosure as hereinafter claimed. It is also understood that various theories as to why the disclosure works are not intended to be limiting.

The present disclosure further relates to methods of treating or preventing an acute inflammatory response, e.g., a cytokine storm in a coronavirus patient, by administering an interleukin-6 (IL-6) inhibitor.

Interleukin-6 (IL-6) is one of the main mediators of inflammatory and immune response initiated by infection or injury, and increased levels of IL-6 are found in more than one half of patients with COVID-19. Levels of IL-6 appear to be associated with inflammatory response, respiratory failure, needing for mechanical ventilation and/or intubation and mortality in COVID-19 patients. The present disclosure is based on the discovery that IL-6 inhibitors may have therapeutic utility in the treatment of coronavirus symptoms, in particular in reducing inflammation and preventing cytokine storms in patients with coronavirus infections, in particular SARS-CoV19.

Thus, in some embodiments, the present disclosure relates to a method of treating or alleviating at least one symptom of a coronavirus infection in a subject, by administering to the subject a therapeutically effective amount of an inteleukin-6 (IL-6) inhibitor. In some embodiments, the symptom is selected from the group consisting of fever, cough, tiredness, sore throat, diarrhea, conjunctivitis, headache, loss of taste, loss of smell, rash, difficulty breathing, shortness of breath, chest pain, chest pressure, Acute Respiratory Distress Syndrome (ARDS) and organ failure. In some embodiments, the subject is a human.

In some embodiments, the present disclosure relates to a method of treating an acute inflammatory condition in a subject infected with a coronavirus, the method comprising the step of administering to the subject a therapeutically effective amount of an inteleukin-6 (IL-6) inhibitor. In some embodiments, the inflammatory condition comprises a cytokine storm. In some embodiments, the subject is a human.

In some embodiments, the present disclosure relates to a method of preventing a cytokine storm in a subject infected with a coronavirus, the method comprising the step of administering to the subject a therapeutically effective amount of an inteleukin-6 (IL-6) inhibitor. In some embodiments, the subject is a human.

In some embodiments, the present disclosure relates to a method of reducing or arresting viral load in a subject infected with a coronavirus, the method comprising the step of administering to the subject a therapeutically effective amount of an inteleukin-6 (IL-6) inhibitor. In some embodiments, the subject is a human.

In some embodiments, the coronavirus is a severe acute respiratory syndrome coronavirus (SARS-CoV). In some embodiments, the coronavirus is a novel virus 2019-nCoV (SARS-CoV-19). In some embodiments, the coronavirus is a Middle East respiratory syndrome coronavirus (MFRS-CoV). In one preferred embodiment, the coronavirus is SARS-CoV-19.

In some embodiments, the IL-6 inhibitor is 3-methoxy-N-(3-(1-methyl-1H-pyrazol-5-yl)-4-(2-morpholinoethoxy)phenyl)benzamide (temanogrel) or salts thereof. In other embodiments, the IL-6 inhibitor is N-(3-(1H-pyrazol-5-yl)phenyl)-3-(3-(piperidin-1-yl)propoxy)benzamide or salts thereof. In other embodiments, the IL-6 inhibitor is N-(3-(1H-pyrazol-5-yl)phenyl)-3-(2-(piperidin-1-yl)ethoxy)benzamide. In other embodiments, the IL-6 inhibitor is a compound of formula (I). Combinations of IL-6 inhibitors may also be used in the methods of the present disclosure.

In some embodiments, IL-6 inhibitor is administered according to a dose regimen selected from the group consisting of once daily (q.d.), twice daily (b.i.d.) thrice daily (t.i.d.), once a week, twice a week, three times a week, once every 2 weeks, once every three weeks, or once a month.

In some embodiments, the IL-6 inhibitor is administered in a pharmaceutical composition, wherein the composition further comprises at least one pharmaceutically acceptable excipient.

In some embodiments, the IL-6 inhibitor is administered in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream.

In some embodiments, the pharmaceutical composition is formulated for oral, topical, mucosal, intranasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural, sublingual oral, intranasal, intravenous, intraarterial, intrathecal, vaginal, rectal or subcutaneous administration.

In some embodiments, the present disclosure relates to pharmaceutical composition in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream, the composition comprising a IL-6 inhibitor and at least one topically acceptable excipient, wherein the IL-6 inhibitor is 3-methoxy-N-(3-(1 -methyl-1H-pyrazo1-5-yl)-4-(2-morpholinoethoxy)phenyl)benzamide temanogrel), N-(3-(1H-pyrazol-5-yl)phenyl)-3-(3-(piperidin-1-yl)propoxy)benzamide, N-(3-(1H-pyrazol-5-yl)phenyl)-3-(2-(piperidin-1-yl)ethoxy)benzamide; or a compound of formula (I), and salts and combinations thereof.

### Interleukin-6 (IL-6) and IL-6 Inhibitors

Interleukin-6 (IL-6) is a cytokine responsible for organ development, inflammation, and immune responses. IL-6 is associated with proinflammatory responses, particularly when dysregulated or continually synthesized. Many cytokines take part in the “cytokine storm” in COVID-19 patients, including IL-6, IL-1, IL-2, IL-10, TNF-α and IFN-γ; however, a crucial role appears to be played by IL-6, whose increased levels in the serum have been correlated with respiratory failure, ARDS, and adverse clinical outcomes. Additionally, exaggerated synthesis of IL-6 in the lungs, secondary to the presence of the nucleocapsid protein of severe acute respiratory syndrome coronavirus 2, has been linked to cytokine release syndrome (CRS) in critically ill COVID-19 patients.

Accordingly, IL-6 inhibitors may be useful therapeutic candidates for treating COVID-19 patients, in particular for ameliorating severe damage to lung tissue caused by cytokine release in patients with serious COVID-19 infections.

The term “IL-6 inhibitor” or “IL-6 antagonist”, as used herein interchangeably, refers to a compound which prevents IL-6 from binding to IL-6 receptors, thus impeding the formation of immune signaling complexes on cell surfaces.

For example, a compound for use in the methods of the present disclosure is art IL-6 inhibitor characterized by the ability to inhibit the binding of IL-6 to the IL-6 receptor, e.g., with an IC50 of 25 μM or less. By way of illustration, an IL-6 inhibitor inhibits binding of IL-6 to IL-6 receptor with an IC50 of about 25 μM, 15 μM, 10 μM, 7.5 μM, 5 μM, 2.5 μM, 1.5 μM, 1 μM, 0.5 μM, 0.25 μM, 0.1 μM, 0.01 μM, or about 0.001 μM.

In some embodiments, the compound is Temanogrel, which is chemically designated 3-methoxy-N-(3-(1-methyl-1H-pyrazol-5-yl)-4-(2-morpholinoethoxy)phenyl)benzamide. Temanogrel is an inverse agonist of the serotonin 2A receptor which was previously developed for the treatment of thrombotic diseases/Acute Coronary Syndrome. The compound has Phase 1 trials in healthy volunteers. The structure of Temanogrel is represented below.

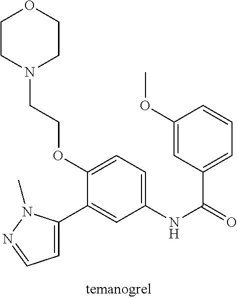 [SMILES: COc1cccc(C(=O)Nc2ccc(OCCN3CCOCC3)c(-c3ccnn3C)c2)c1]

In some embodiments, Temanogrel is administered orally. In some embodiments, Temanogrel is administered orally at a dose between 10 mg and 500 mg, e.g., 10 mg, 20 mg, 30 mg, 40 mg, 50 mg, 80 mg, 100 mg, 150 mg, 200 mg, 250 mg, 300 mg, 350 mg, 400 mg, 450 mg or 500 mg.

Temanogrel and structurally related compounds are described in PCT International Patent Application WO 2006/055734. Structurally related compounds are disclosed in PCT International Patent Application WO 2019/165158. The contents of each of these references are hereby incorporated by reference for all purposes and the specific purposes identified herein. In some embodiments, the compound is N-(3-(1H-pyrazol-5-yl)phenyl)-3-(3-(piperidin-1-yl)propoxy)benzamide, which is represented by the following chemical structure:

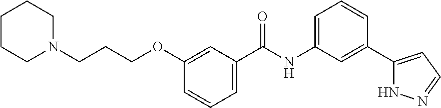 [SMILES: O=C(Nc1cccc(-c2ccn[nH]2)c1)c1cccc(OCCCN2CCCCC2)c1]

In some embodiments, the compound is N-(3-(1H-pyrazol-5-yl)phenyl)-3-(2-(piperidin-1-yl)ethoxy)benzamide, which is represented by the following chemical structure:

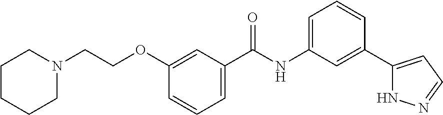 [SMILES: O=C(Nc1cccc(-c2ccn[nH]2)c1)c1cccc(OCCN2CCCCC2)c1]

Additional compounds suitable for use in the methods of the present invention are represented by the structure of formula (I):

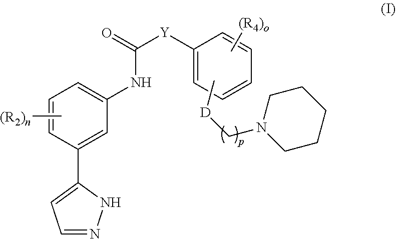

wherein
each R₂ is independently halo, cyano, —OR₅, NO₂, alkyl, haloalkyl, hydroxyalkyl, alkoxyalkyl, or —NR₆R₇;
each R₄ is independently halo, cyano, —OR₅, N₂, alkyl, haloalkyl, hydroxyalkyl, alkoxyalkyl, or —NR6R7
each R₆ is independently hydrogen or optionally substituted alkyl;
each R₇ is independently hydrogen or optionally substituted alkyl;
D is a bond, —O—, —S—, or —N(R₆)—;
Y is a bond, —O—, —S—, or —N(R₆)—;
n 0, 1, 2, 3, or 4;
o is 0, 1, 2, 3, or 4; and
p is 0, 1, 2, 3, or 4.

### Methods of Treatment

In certain embodiments, the compositions and methods of the present disclosure are useful for the prevention and/or treatment of symptoms of SARS-CoV-19 infections. In certain embodiments, the compositions and methods of the present disclosure are useful for the prevention and/or treatment of acute inflammatory responses. In certain embodiments, the compositions and methods of the present disclosure are useful for the prevention and/or treatment of acute inflammatory responses, e.g., cytokine storms that are associates with a coronavirus infection.

The present disclosure is based on the discovery that IL-6 inhibitors may have therapeutic utility in the treatment of coronavirus symptoms, in particular in preventing cytokine storms in critical patients with coronavirus infections, in particular SARS-CoV-19.

Thus, in some embodiments, IL-6 inhibitors may prevent onset of severe SARS-CoV-19 symptoms. For example, IL-6 inhibitors may prevent or ameliorate the hyper-inflammatory response in patients with SARS-CoV-19 pneumonia and prevent or ameliorate progress to cytokine storm. Successful intervention with IL-6 inhibitors may reduce life-threatening complications of SARS-CoV-19, including severe respiratory symptoms that often necessitate further medical intervention such as mechanical intervention.

Thus, in some embodiments, the present disclosure relates to a method of treating or alleviating at least one symptom of a coronavirus infection in a subject, by administering to the subject a therapeutically effective amount of an inteleukin-6 (IL-6) inhibitor. In some embodiments, the subject is a human.

In some embodiments, the symptom is fever. In other embodiments, the symptom is cough. In other embodiments, the symptom is dry cough. In other embodiments, the symptom is tiredness. In other embodiments, the symptom is sore throat. In other embodiments, the symptom is diarrhea. In other embodiments, the symptom is conjunctivitis. In other embodiments, the symptom is headache. In other embodiments, the symptom is loss of taste. In other embodiments, the symptom is loss of smell. In other embodiments, the symptom is a rash. In other embodiments, the symptom is difficulty breathing. In other embodiments, the symptom is shortness of breath. In other embodiments, the symptom is chest pain. In other embodiments, the symptom is chest pressure. In other embodiments, the symptom is Acute Respiratory Distress Syndrome (ARDS). In other embodiments, the symptom is organ failure. In other embodiments, the symptom is multiple organ failure. In other embodiments, the symptom is any combination of the foregoing.

In some embodiments, the present disclosure relates to a method of treating an acute inflammatory condition in a subject infected with a coronavirus, the method comprising the step of administering to the subject a therapeutically effective amount of an inteleukin-6 (IL-6) inhibitor. In some embodiments, the inflammatory condition comprises a cytokine storm. In some embodiments, the subject is a human.

In some embodiments, the present disclosure relates to a method of preventing cytokine storm in a subject infected with a coronavirus, the method comprising the step of administering to the subject a therapeutically effective amount of an inteleukin-6 (IL-6) inhibitor. In some embodiments, the subject is a human.

In some embodiments, the present disclosure relates to a method of reducing or arresting viral load in a subject infected with a coronavirus, the method comprising the step of administering to the subject a therapeutically effective amount of an inteleukin-6 (IL-6) inhibitor. In some embodiments, the subject is a human.

Viral load can be measured by any viral diagnostic equipment or technique known in the art. A wide variety of samples can be used for virological testing. Such samples include, but are not limited to, upper respiratory swabs (nasopharyngeal swabs, nasopharyngeal wash/aspirate, oropharyngeal swabs, saliva) and lower respiratory specimens (sputum, bronchoalveolar lavage, lung tissue), as well as stool, rectal swabs, blood, skin, urine, semen, faeces, cerebrospinal fluid, tissue (e.g., biopsies), and the like. Techniques for measuring viral load include, but are not limited to, nucleic acid amplification-based tests (NATs) or non-nucleic acid-based tests. Examples of NATs include, but are not limited to, PCR (polymerase chain reaction), reverse transcription polymerase chain reaction (RT-PCR), and nucleic acid sequence-based amplification (NASBA). Viral load is typically reported as copies the virus in a milliliter (mL) of blood. Changes in viral load are usually reported as a log change (in powers of 10). For example, a three-log increase in viral load (3 log10) is an increase of 10³ or 1,000 times the previously reported level, while a drop from 500,000 to 500 copies would be a three-log-drop.

In one embodiment, the subject is infected with a coronavirus. In some embodiments, the coronavirus is selected from the group consisting of 229E (alpha coronavirus), NL63 (alpha coronavirus), OC43 (beta coronavirus), HKU1 (beta coronavirus), MERS-CoV (beta coronavirus that causes Middle East Respiratory Syndrome, or MERS), SARS-CoV (the beta coronavirus that causes severe acute respiratory syndrome, or SARS) SARS-CoV-2 (the novel coronavirus that causes coronavirus disease 2019, or COVID-19, also referred to herein as SARS-Covid-19). In some embodiments, the coronavirus is a severe acute respiratory syndrome coronavirus (SARS-CoV). In some embodiments, the coronavirus is a novel virus 2019-nCoV (SARS-CoV-19). In some embodiments, the coronavirus is a Middle East respiratory syndrome coronavirus (MERS-CoV). In one preferred embodiment, the coronavirus is SARS-CoV-19.

### Pharmaceutical Compositions

The present disclosure thus provides pharmaceutical compositions comprising IL-6 inhibitors and a pharmaceutically acceptable carrier. The compounds of the present disclosure can be formulated as pharmaceutical compositions and administered to a mammalian host, such as a human patient, in a variety of forms adapted to the chosen route of administration.

Routes of administration include, but are not limited to oral, topical, mucosal, nasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural and sublingual administration.

As used herein, the term “administering” generally refers to any and all means of introducing compounds described herein to the host subject. Compounds described herein may be administered in unit dosage forms and/or compositions containing one or more pharmaceutically-acceptable carriers, adjuvants, diluents, excipients, and/or vehicles, and combinations thereof.

As used herein, the terms “composition” generally refers to any product comprising more than one ingredient, including the compounds described herein. It is to be understood that the compositions described herein may be prepared from compounds described herein or from salts, solutions, hydrates, solvates, and other forms of the compounds described herein. It is appreciated that the compositions may be prepared from various amorphous, non-amorphous, partially crystalline, crystalline, and/or other morphological forms of the compounds described herein, and the compositions may be prepared from various hydrates and/or solvates of the compounds described herein. Accordingly, such pharmaceutical compositions that recite compounds described herein include each of, or any combination of, or individual forms of, the various morphological forms and/or solvate or hydrate forms of the compounds described herein.

In some embodiments, the IL-6 inhibitors may be systemically (e.g., orally) administered in combination with a pharmaceutically acceptable vehicle such as an inert diluent or an assimilable edible carrier. For oral therapeutic administration, the active compound may be combined with one or more excipients and used in the form of ingestible tablets, buccal tablets, sublingual tablets, troches, capsules, elixirs, suspensions, syrups, wafers, and the like. The percentage of the compositions and preparations may vary and may be between about 1 to about 99% weight of the active ingredient(s) and excipients such as, but not limited to a binder, a filler, a diluent, a disintegrating agent, a lubricant, a surfactant, a sweetening agent; a flavoring agent, a colorant, a buffering agent, anti-oxidants, a preservative, chelating agents (e.g., ethylenediaminetetraacetic acid), and agents for the adjustment of tonicity such as sodium chloride.

Suitable binders include, but are not limited to, polyvinylpyrrolidone, copovidone, hydroxypropyl methylcellulose, starch, and gelatin.

Suitable fillers include, but are not limited to, sugars such as lactose, sucrose, mannitol or sorbitol and derivatives therefore (e.g. amino sugars), ethylcellulose, microcrystalline cellulose, and silicified microcrystalline cellulose.

Suitable diluents include, but are not limited to, dicalcium phosphate dihydrate, sugars, lactose, calcium phosphate, cellulose, kaolin, mannitol, sodium chloride, and dry starch.

Suitable disintegrants include, but are not limited to, pregelatinized starch, crospovidone, crosslinked sodium carboxymethyl cellulose and combinations thereof. Suitable lubricants include, but are not limited to, sodium stearyl fumarate, stearic acid, polyethylene glycol or stearates, such as magnesium stearate.

Suitable surfactants or emulsifiers include, but are not limited to, polyvinyl alcohol (PVA), polysorbate, polyethylene glycols, polyoxyethylene- polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil.

Suitable flavoring agents and sweeteners include, but are not limited to, sweeteners such as sucralose and synthetic flavor oils and flavoring aromatics, natural oils, extracts from plants, leaves, flowers, and fruits, and combinations thereof. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot.

Suitable colorants include, but are not limited to, alumina (dried aluminum hydroxide), annatto extract, calcium carbonate, canthaxanthin, caramel, β-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide.

Suitable buffering or pH adjusting agent include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide and magnesium hydroxide. Suitable tonicity enhancing agents include, but are not limited to, ionic and non-ionic agents such as, alkali metal or alkaline earth metal halides, urea, glycerol, sorbitol, mannitol, propylene glycol, and dextrose.

Suitable wetting agents include, but are not limited to, glycerin, cetyl alcohol, and glycerol monostearate.

Suitable preservatives include, but are not limited to, benzalkonium chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol, chlorohexidine, and polyhexamethylene biguanide.

Suitable antioxidants include, but are not limited to, sorbic acid, ascorbic acid, ascorbate, glycine, α-tocopherol, butylated hydroxyanisole (BHA), and butylated hydroxytoluene (BHT).

The IL-6 inhibitors of the present disclosure may also be administered via infusion or injection (e.g., using needle (including microneedle) injectors and/or needle-free injectors).

Solutions of the active composition can be aqueous, optionally mixed with a nontoxic surfactant and/or may contain carriers or excipients such as salts, carbohydrates and buffering agents (preferably at a pH of from 3 to 9), and, for some applications, they may be more suitably formulated as a sterile non-aqueous solution or as a dried form to be used in conjunction with a suitable vehicle such as sterile, pyrogen-free water or phosphate-buffered saline. For example, dispersions can be prepared in glycerol, liquid polyethylene glycols, triacetin, and mixtures thereof and in oils. The preparations may further contain a preservative to prevent the growth of microorganisms.

The pharmaceutical compositions may be formulated for parenteral administration (e.g., subcutaneous, intravenous, intra-arterial, transdermal, intraperitoneal or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Water is a preferred carrier when the pharmaceutical composition is administered intravenously. Saline solutions and aqueous dextrose and glycerol solutions can also be employed as liquid carriers, particularly for injectable solutions. Oils such as petroleum, animal, vegetable, or synthetic oils and soaps such as fatty alkali metal, ammonium, and triethanolamine salts, and suitable detergents may also be used for parenteral administration. Further, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. Suitable preservatives include e.g. sodium benzoate, benzoic acid, and sorbic acid. Suitable antioxidants include e.g. sulfites, ascorbic acid and □-tocopherol.

The preparation of parenteral compounds/compositions under sterile conditions, for example, by lyophilization, may readily be accomplished using standard pharmaceutical techniques well known to those skilled in the art.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. In one embodiment, the compositions are administered by the oral or nasal respiratory route for local or systemic effect. Compositions in pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, orally or nasally, from devices that deliver the formulation in an appropriate manner.

In yet another embodiment, the composition is prepared for topical administration, e.g. as an ointment, a gel, a drop, a patch or a cream. For topical administration to body surfaces using, for example, creams, gels, drops, ointments and the like, the compounds of the present disclosure can be prepared and applied in a physiologically acceptable diluent with or without a pharmaceutical carrier. Adjuvants for topical or gel base forms may include, for example, sodium carboxymethylcellulose, polyacrylates, polyoxyethylene-polyoxypropylene-block polymers, polyethylene glycol, wood wax alcohols, isostearic acid, cetyl alcohol, stearyl alcohol, white petrolatum, polysorbate 60, sorbitan monostearate, glycerin, xanthan gum, water, benzyl alcohol, methylparaben, and propylparaben. Additional additives may be selected from the group consisting of waxes, soaps, sorbitan esters, fatty acids, fatty acid esters, fatty acid oils, borates, cresol, chlorocresol, cellulose, methylcellulose, hydroxypropylcellulose, acacia, and the like. Examples of suitable topical dosage forms may be found in e.g., Tarun Garg, Goutam Rath & Amit K. Goyal (2015) Comprehensive review on additives of topical dosage forms for drug delivery, Drug Delivery, 22:8, 969-987, the contents of which are hereby incorporated by reference in their entirety.

Alternative formulations include nasal sprays, liposomal formulations, slow-release formulations, pumps delivering the drugs into the body (including mechanical or osmotic pumps) controlled-release formulations and the like, as are known in the art.

### Doses

As used herein, the term “therapeutically effective dose” means (unless specifically stated otherwise) a quantity of a compound which, when administered either one time or over the course of a treatment cycle affects the health, wellbeing or mortality of a subject (e.g., delays the onset of and/or reduces the severity of one or more of the symptoms associated with a coronavirus, e.g., SARS-Covid-19.

A IL-6 inhibitors described herein can be present in a composition in an amount of about 0.001 mg, about 0.005 mg, about 0.01 mg, about 0.02 mg, about 0.03 mg, about 0.04 mg, about 0.05 mg, about 0.06 mg, about 0.07 mg, about 0.08 mg, about 0.09 mg about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 0.5 mg, about 10 mg, about 10.5 mg, about 11 mg, about 12 mg, about 12.5 mg, about 13 mg, about 13.5 mg, about 14 mg, about 14.5g, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg.

A IL-6 inhibitors described herein described herein can be present in a composition in a range of from about 0.1 mg to about 100 mg; 0.1 mg to about 75 mg; from about 0.1 mg to about 50 mg; from about 0.1 mg to about 25 mg; from about 0.1 mg to about 10 mg; 0.1 mg to about 7.5 mg, 0.1 mg to about 5 mg; 0.1 mg to about 2.5 mg; from about 0.1 mg to about 1 mg; from about 0.5 mg to about 100 mg; from about 0.5 mg to about 75 mg; from about 0.5 mg to about 50 mg; from about 0.5 mg to about 25 mg; from about 0.5 mg to about 10 mg; from about 0.5mg to about 5 mg, from about 0.5mg to about 2.5 mg; from about 0.5 mg to about 1 mg; from about 1 mg to about 100 mg; from about 1 mg to about 75 mg; from about 0.1 mg to about 50 mg; from about 0.1 mg to about 25 mg; from about 0.1 mg to about 10 mg; from about 0.1 mg to about 5 mg; from about 0.1 mg to about 2.5 mg; from about 0.1 mg to about 1 mg.

### Dosing Regimens

The compounds described herein can be administered by any dosing schedule or dosing regimen as applicable to the patient and/or the condition being treated. Administration can be once a day (q.d.), twice a day (b.i.d.), thrice a day (t.i.d.), once a week, twice a week, three times a week, once every 2 weeks, once every three weeks, or once a month twice, and the like.

In some embodiments, the IL-6 inhibitor is administered for a period of at least one day. In other embodiments, the IL-6 inhibitor is administered for a period of at least 2 days. In other embodiments, the IL-6 inhibitor is administered for a period of at least 3 days. In other embodiments, the IL-6 inhibitor is administered for a period of at least 4 days. In other embodiments, the IL-6 inhibitor is administered for a period of at least 5 days. In other embodiments, the IL-6 inhibitor is administered for a period of at least 6 days. In other embodiments, the IL-6 inhibitor is administered for a period of at least 7 days. In other embodiments, the IL-6 inhibitor is administered for a period of at least 10 days. In other embodiments, the IL-6 inhibitor is administered for a period of at least 14 days. In other embodiments, the IL-6 inhibitor is administered for a period of at least one month. In some embodiments, the IL-6 inhibitor is administered chronically for as long as the treatment is needed.

The present subject matter described herein will be illustrated more specifically by the following non-limiting examples, it being understood that changes and variations can be made therein without deviating from the scope and the spirit of the disclosure as hereinafter claimed. It is also understood that various theories as to why the disclosure works are not intended to be limiting.

IL-6 inhibitor example embodiments:
1. A method of treating or alleviating at least one symptom of a coronavirus infection in a subject, the method comprising the step of administering to the subject a therapeutically effective amount of an inteleukin-6 (IL-6) inhibitor.
2. The method according to embodiment 1, wherein the symptom is selected from the group consisting of fever, cough, tiredness, sore throat, diarrhea, conjunctivitis, headache, loss of taste, loss of smell, rash, difficulty breathing, shortness of breath, chest pain, chest pressure, Acute Respiratory Distress Syndrome (ARDS) and organ failure.
3. A method of preventing or treating an acute inflammatory condition in a subject infected with a coronavirus, the method comprising the step of administering to the subject a therapeutically effective amount of an inteleukin-6 (IL-6) inhibitor.
4. The method according to embodiment 3, wherein the inflammatory condition comprises a cytokine storm.
5. A method of preventing or treating a cytokine storm in a subject infected with a coronavirus, the method comprising the step of administering to the subject a therapeutically effective amount of an inteleukin-6 (IL-6) inhibitor.
6. A method of reducing or arresting viral load in a subject infected with a coronavirus, the method comprising the step of administering to the subject a therapeutically effective amount of an inteleukin-6 (IL-6) inhibitor.
7. The method according to any one of embodiments 1 to 6, wherein the coronavirus is selected from the group consisting of severe acute respiratory syndrome corona virus (SARS-CoV), novel virus 2019-nCoV (SARS-CoV-19), and the Middle East respiratory syndrome coronavirus (MERS-CoV).
8. The method according to embodiment 7, wherein the corona virus is SARS-CoV-19.
9. The method according to any one of the preceding embodiments, wherein the IL-6 inhibitor is selected from the group consisting of:
   3-methoxy-N-(3-(1-methyl-1H-pyrazol-5-yl)-4-(2-morpholinoethoxy) phenyl)benzamide (temanogrel);
   N-(3-(1H-pyrazol-5-yl)phenyl)-3-(2-(piperidin-1-yl)ethoxy)benzamide; and
   N-(3-(1H-pyrazol-5-yl)phenyl)-3-(3-(piperidin-1-yl)propoxy)benzamide;
   and salts and any combinations thereof.
10. The method according to any one of the preceding embodiments, wherein the IL-6 inhibitor is represented by the structure of formula (I):

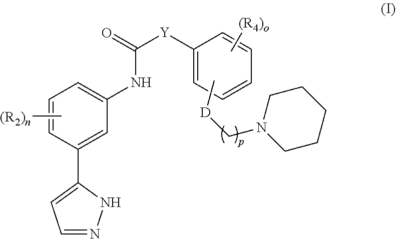

wherein
each R₂ is independently halo, cyano, —OR₅, NO₂, alkyl, haloalkyl, hydroxyalkyl, alkoxyalkyl, or —NR₆R₇;
each R 4 is independently halo, cyano, —OR₅, NO₂, alkyl, haloalkyl, hydroxyalkyl, alkoxyalkyl, or —NR₆R₇;
each R 6 is independently hydrogen or optionally substituted alkyl;
each R 7 is independently hydrogen or optionally substituted alkyl;
D is a bond, —O—, —S—, or —N(R₆)—;
Y is a bond, —O—, —S—, or —N(R₆)—; and
n is 0, 1, 2, 3, or 4;
o is 0, 1, 2, 3, or 4; and
p 0, 1, 2, 3, or 4.
11. The method according to any one of the preceding embodiments, wherein the IL-6 inhibitor is administered according to a dosing regimen selected from the group consisting of once daily (q.d.), twice daily (b.i.d.) thrice daily (t.i.d.), once a week, twice a week, three times a week, once every 2 weeks, once every three weeks, or once a month.
12. The method according to any one of the preceding embodiments, wherein the IL-6 inhibitor is administered in a pharmaceutical composition, wherein the composition further comprises at least one pharmaceutically acceptable excipient.
13. The method according to embodiment 12, wherein the IL-6 inhibitor is administered in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream.
14. The method according to embodiments 12 or 13, wherein the pharmaceutical composition is formulated for oral, topical, mucosal, intranasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural, sublingual oral, intranasal, intravenous, intraarterial, intrathecal, vaginal, rectal or subcutaneous administration.
15. The method according to any one of the preceding embodiments, wherein the subject is a human.
16. A pharmaceutical composition in a form selected from the group consisting of a solution, a suspension, a syrup, an emulsion, a dispersion, a tablet, a pill. a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream, the composition comprising a IL-6 inhibitor and at least one pharmaceutically acceptable excipient, wherein the IL-6 inhibitor is selected from the group consisting of
   3-methoxy-N-(3-(1-methyl-1H-pyrazol-5-yl)-4-(2-morpholinoethoxy) phenyl)benzamide (temanogrel);
   N-(3-(1H-pyrazol-5-yl)phenyl)-3-(2-(piperidin-1 -yl)ethoxy)benzamide; and
   N-(3-(1H-pyrazol-5-yl)phenyl)-3-(3-(piperidin-1-yl)propoxy)benzamide;
   and salts and any combinations thereof.
17. A pharmaceutical composition in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream, the composition comprising a IL-6 inhibitor and at least one pharmaceutically acceptable excipient, wherein the IL-6 inhibitor is represented by the structure of formula (I):

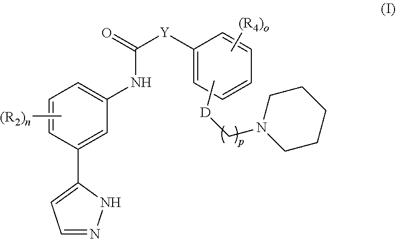

wherein
each R₂ is independently halo, cyano, —OR₅, NO₂, alkyl, haloalkyl, hydroxyalkyl, alkoxyalkyl, or —NR₆R₇;
each R₄ is independently halo, cyano, —OR₅, NO₂, alkyl, haloalkyl, hydroxyalkyl, alkoxyalkyl, or —NR₆R₇;
each R₆ is independently hydrogen or optionally substituted alkyl;
each R₇ is independently hydrogen or optionally substituted alkyl;
D is a bond, —O—, —S—, or —N(R₆)—;
Y is a bond, —O—, —S—, or —N(R₆)—; and
n is 0, 1, 2, 3, or 4;
o is 0, 1, 2, 3, or 4; and
p is 0, 1 2, 3, or4.

The present disclosure further relates to methods of treating or preventing an acute inflammatory response, e.g., a cytokine storm in a coronavirus patient, by administering a CXC chemokine receptor 3 (CXCR3) and/or a CXC chemokine receptor 4 (CXCR4).

CXCR3 is a chemokine receptor that binds CXC-chemokines and is restrictively expressed in activated T cells. Binding of chemokines to CXCR3 induces cellular responses that are involved in leukocyte traffic, e.g., integrin activation, cytoskeletal changes and chemotactic migration. CXCR4 is a chemokine receptor that has been shown to be involved in a number of pathological conditions, including cancer and inflammatory diseases, e.g., including autoimmune diseases, rheumatoid arthritis, inflammatory bowel disease, ischemic injuries and lung diseases.

The present disclosure is based on the discovery that CXCR3/CXCR4 antagonists may have therapeutic utility in the treatment of coronavirus symptoms, in particular in reducing inflammation and preventing cytokine storms in patients with coronavirus infections, in particular SARS-CoV-19.

Thus, in some embodiments, the present disclosure relates to a method of treating or alleviating at least one symptom of a coronavirus infection in a subject, by administering to the subject a therapeutically effective amount of a CXCR3 or CXCR4 antagonist. In some embodiments, the symptom is selected from the group consisting of fever, cough, tiredness, sore throat, diarrhea, conjunctivitis, headache, loss of taste, loss of smell, rash, difficulty breathing, shortness of breath, chest pain, chest pressure, Acute Respiratory Distress Syndrome (ARDS) and organ failure. In some embodiments, the subject is a human.

In some embodiments, the present disclosure relates to a method of treating an acute inflammatory condition in a subject infected with a coronavirus, the method comprising the step of administering to the subject a therapeutically effective amount of a CXCR3 or

CXCR4 antagonist. In some embodiments, the inflammatory condition comprises a cytokine storm. In some embodiments, the subject is a human.

In some embodiments, the present disclosure relates to a method of preventing a cytokine storm in a subject infected with a coronavirus, the method comprising the step of administering to the subject a therapeutically effective amount of a CXCR3 or CXCR4 antagonist. In some embodiments, the subject is a human.

In some embodiments, the present disclosure relates to a method of reducing or arresting viral load in a subject infected with a coronavirus, the method comprising the step of administering to the subject a therapeutically effective amount of a CXCR3 or CXCR4 antagonist. In some embodiments, the subject is a human.

In some embodiments, the coronavirus is a severe acute respiratory syndrome coronavirus (SARS-CoV). In some embodiments, the coronavirus is a novel virus 2019-nCoV (SARS-CoV-19). In some embodiments, the coronavirus is a Middle East respiratory syndrome coronavirus (MERS-CoV). In one preferred embodiment, the coronavirus is SARS-CoV-19.

In some embodiments the CXCR3 or CXCR4 antagonist is selected from the group consisting of (4-chlorophenyl)(4-(2-ethyl-4-(5-(5-(ethylamino)-1,3,4-oxadiazol-2-yl)-3-methylpyrazin-2-yl)-5-methylpiperazin-1-yl)piperidin-1- yl)methanone (PS-386113); N-(1-(3-(4-ethoxyphenyl)-4-oxo-3,4-dihydropyrido[2,3-d]pyrimidin-2-yl)ethyl)-N-(pyridin-3-ylmethyl)-2-(4-(trifluoromethoxy)phenyl)acetamide (AMG-487): and salts and any combinations thereof.

In some embodiments, the CXCR3 or CXCR4 antagonist is selected from the group consisting of a compound of any one of Table 1 and Table 2.

In some embodiments, CXCR3 or CXCR4 antagonist is administered according to a dose regimen selected from the group consisting of once daily (q.d.), twice daily (b.i.d.) thrice daily (t.i.d.), once a week, twice a week, three times a week, once every 2 weeks, once every three weeks, or once a month.

In some embodiments, the CXCR3 or CXCR4 antagonist is administered in a pharmaceutical composition, wherein the composition further comprises at least one pharmaceutically acceptable excipient.

In some embodiments, the CXCR3 or CXCR4 antagonist is administered in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream.

In some embodiments, the pharmaceutical composition is formulated for oral, topical, mucosal, intranasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural, sublingual oral, intranasal, intravenous, intraarterial, intrathecal, vaginal, rectal or subcutaneous administration.

In some embodiments, the present disclosure relates to a pharmaceutical composition in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream, the composition comprising a

CXCR3 or CXCR4 antagonist and at least one pharmaceutically acceptable excipient, wherein the CXCR3 or CXCR4 antagonist is selected from the group consisting of 4-chlorophenyl) (4-(2-ethyl-4-(5-(5-(ethylamino)-1,3,4-oxadiazol-2-yl)-3-methylpyrazin-2-yl)-(PS-386113); N-(1-(3-(4-ethoxyphenyl)-4-oxo-3,4-dihydropyrido[2,3-d]pyrimidin-2-yl)ethyl)-N-(pyridin-3-ylmethyl)-2-(4-(trifluoromethoxy)phenyl)acetamide (AMG-487); and salts and any combinations thereof and salts and combinations thereof.

In some embodiments, the present invention relates to a pharmaceutical composition in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream, the composition comprising a CXCR3 or CXCR4 antagonist and at least one pharmaceutically acceptable excipient, wherein the CXCR3 or CXCR4 antagonist is selected from the group consisting of a compound of any one of Table C1 and Table C2.

### Chemokine (C-X-C motif) Receptors 3 (CXCR3) and 4 (CXCR4)

Chemokines constitute a family of cytokines that are produced in immune response and inflammation. Pathogenic infection is often accompanied by robust chemokine signalling elicited from infected cells, which contributes to both innate and adaptive immune responses that control growth of the invading pathogen. Chemokines share four conserved cysteines, which form disulfide bonds. Based upon this conserved cysteine motif, the family can be divided into distinct branches: CXC, CC, CX3C and XC. In C-X-C chemokines (□-chemokines) the first two conserved cysteines are separated by an intervening residue.

Chemokine receptors are members of the G protein-coupled receptor superfamily. These receptors are involved in cell movement, and thus play a critical role in several physiological and pathological situations that require regulation of cell positioning.

CXCR3 is a chemokine receptor that binds the CXC-chemokines CXCL9 (MIG), CXCL10 (IP-10), and CXCL11 (I-TAC). CXCR3 is restrictively expressed in activated T cells, preferentially Th1 cells. Binding of chemokines to this protein induces cellular responses that are involved in leukocyte traffic, most notably integrin activation, cytoskeletal changes and chemotactic migration. Lymphocytes expressing a CXCR3 receptor as a result of activation can be recruited into inflammatory lesions or sites of infection by IP-10, MIG and/or I-TAC, which can be induced locally by interferon-gamma. Thus, CXCR3 plays a role in the selective recruitment of lymphocytes, and hence plays a role in inflammatory responses.

The chemokine receptor CXCR4 is widely expressed throughout the human body during embryonic development and adult life, with uniquely high-expression levels in the hematopoietic system. Its cognate ligand, the chemokine CXCL12 (also named stromal cell-derived factor-1α, SDF-1α), is mainly expressed in the bone marrow, lymph nodes, lung, heart, thymus and liver. CXCR4 receptor has also been found to be involved in a variety of diseases including mediating HIV-1 entry into T cells as a co-receptor, rheumatoid arthritis, atherosclerosis, vascular remodelling after injury, atherosclerotic plaque destabilization and aneurysm formation.

The present disclosure is based on the discovery that CXC receptor 3 (CXCR3)/CXC receptor 4 (CXCR4) antagonists may have therapeutic utility in the treatment of coronavirus symptoms, in particular in reducing inflammation and preventing cytokine storms in patients with coronavirus infections, in particular SARS-CoV-19.

### CXCR3 and CXCR4 Antagonists

As used herein, the term “CXCR antagonist” refers to a substance (e.g., a small molecule) that blocks a CXCR receptor and prevents its activation. In some embodiments, the CXCR antagonist inhibits the binding of CXCR to its chemokine ligand which in turn prevents downstream effects. For example, a CXCR3 antagonist may block binding of the chemokines CXCL9 (MIG), CXCL10 (IP-10), and/or CXCL11 (I-TAC) to CXCR3. The CXCR3 antagonist may inhibit the binding of CXCR3 to its chemokine ligand either by binding to CXCR3, or by binding to one of the chemokine ligands, or both. A CXCR4 antagonist may block binding of the chemokine CXCL12 (SDF-1α) to CXCR4. The CXCR4 antagonist may inhibit the binding of CXCR4 to its chemokine ligand either by binding to CXCR4, or by binding to the chemokine ligand, or both.

In some embodiments, a compound for use in the methods of the present disclosure is a CXCR3 antagonist. A CXCR3 antagonist is characterized by the ability to inhibit the activation of a cytokine receptor CXCR3, e.g., with an IC50 of 25 μM or less. By way of illustration, a CXCR3 antagonist inhibits the activation of a cytokine receptor CXCR3 with an IC50 of about 25 μM, 15 μM, 10 μM, 7.5 μM, 5 μM, 2.5 μM, 1.5 μM, 1 μM, 0.5 μM, 0.25 μM, 0.1 μM, 0.01 μM, or about 0.001 μM.

Alternatively, a CXCR3 antagonist blocks binding of the chemokines CXCL9 (MIG), CXCL10 (IP-10), and/or CXCL11 (I-TAC) to CXCR3, e.g., with an IC50 of 25 μM or less. By way of illustration, a CXCR3 antagonist inhibits the binding with an IC50 of about 25 μM, 15 μM, 10 μM, 7.5 μM, 5 μM, 2.5 μM, 1.5 μM, 1 μM, 0.5 μM, 0.25 μM, 0.1 μM, 0.01 μM, or about 0.001 μM.

In other embodiments, a compound for use in the methods of the present disclosure is a CXCR4 antagonist. A CXCR4 antagonist is characterized by the ability to inhibit the activation of a cytokine receptor CXCR4, e.g., with an IC50 of 25 μM or less. By way of illustration, a CXCR4 antagonist inhibits the activation of a cytokine receptor CXCR4 with an IC50 of about 25 μM, 15 μM, 10 μM, 7.5 μM, 5 μM, 2.5 μM, 1.5 μM, 1 μM , 0.5 μM, 0.25 μM, 0.1 μM, 0.01 μM, or about 0.001 μM.

Alternatively, a CXCR4 antagonist blocks binding of the chemokine CXCL12 (SDF-1α) to CXCR4, e.g., with an IC50 of 25 μM or less. By way of illustration, a CXCR4 antagonist inhibits the binding with an IC50 of about 25 μM, 15 μM, 10 μM, 7.5 μM, 5 μM, 2.5 μM, 1.5 μM, 1 μM, 0.5 μM, 0.25 μM, 0.1 μM, 0.01 μM, or about 0.001 μM.

In some embodiments, the CXCR3 antagonist is a heterocyclic substituted piperazine such as PS-386113 or salts thereof, a compound previously developed for the treatment of inflammation. PS-386113 is chemically designated (4-chlorophenyl) (4-(2-ethyl-4-(5-(5-(ethylamino)-1,3,4-oxadiazol-2-yl)-3-methylpyrazin-2-yl)-5- methylpiperazin-1-yl)piperidin-1-yl)methanone, and its structure is shown below:

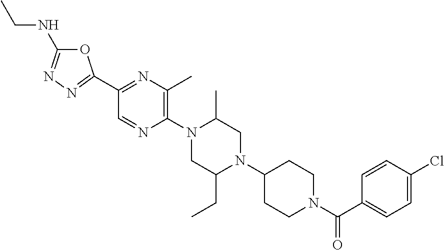 [SMILES: CCNc1nnc(-c2cnc(N3CC(CC)N(C4CCN(C(=O)c5ccc(Cl)cc5)CC4)CC3C)c(C)n2)o1]

PS-386113 and structurally related compounds are described in PCT International Patent Application WO 2006/088837, the contents of which are hereby incorporated by reference for all purposes and the specific purposes identified herein. In some embodiments, such compounds are represented by any one or more of the structures shown in Table 1. Any one of the compounds depicted in Table C1 is suitable for use in the methods of the present disclosure.

TABLE C1
Compound

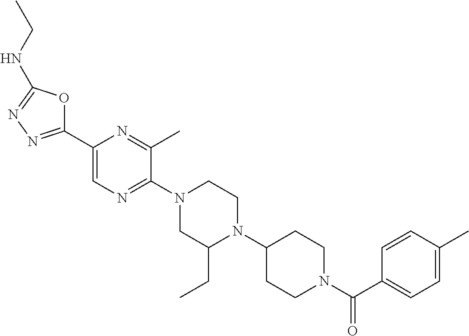 [SMILES: CCNc1nnc(-c2cnc(N3CCN(C4CCN(C(=O)c5ccc(C)cc5)CC4)C(CC)C3)c(C)n2)o1]

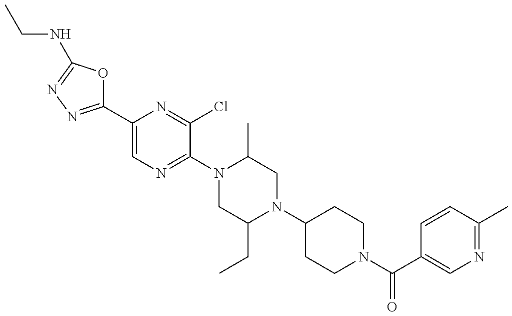 [SMILES: CCNc1nnc(-c2cnc(N3CC(CC)N(C4CCN(C(=O)c5ccc(C)nc5)CC4)CC3C)c(Cl)n2)o1]

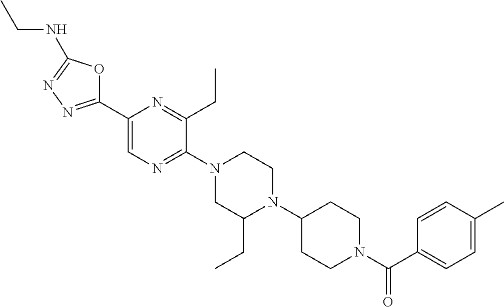 [SMILES: CCNc1nnc(-c2cnc(N3CCN(C4CCN(C(=O)c5ccc(C)cc5)CC4)C(CC)C3)c(CC)n2)o1]

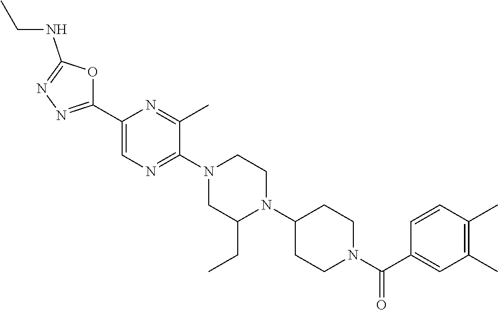 [SMILES: CCNc1nnc(-c2cnc(N3CCN(C4CCN(C(=O)c5ccc(C)c(C)c5)CC4)C(CC)C3)c(C)n2)o1]

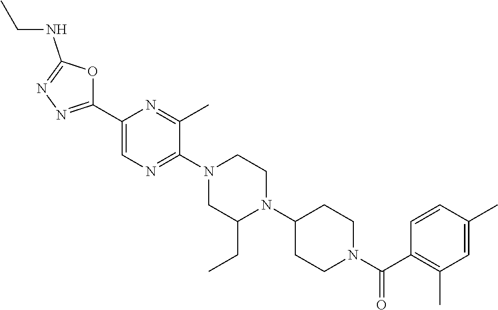 [SMILES: CCNc1nnc(-c2cnc(N3CCN(C4CCN(C(=O)c5ccc(C)cc5C)CC4)C(CC)C3)c(C)n2)o1]

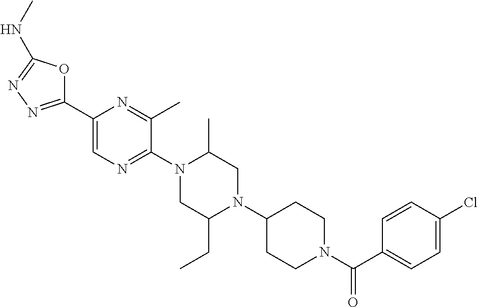 [SMILES: CCC1CN(c2ncc(-c3nnc(NC)o3)nc2C)C(C)CN1C1CCN(C(=O)c2ccc(Cl)cc2)CC1]

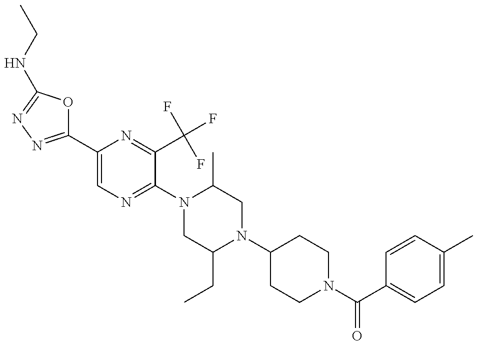 [SMILES: CCNc1nnc(-c2cnc(N3CC(CC)N(C4CCN(C(=O)c5ccc(C)cc5)CC4)CC3C)c(C(F)(F)F)n2)o1]

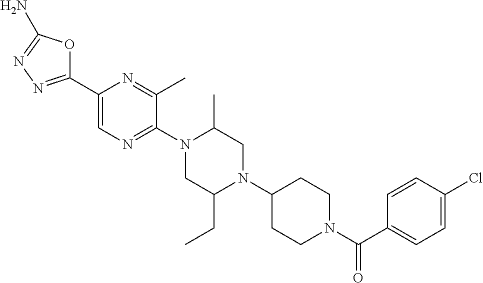 [SMILES: CCC1CN(c2ncc(-c3nnc(N)o3)nc2C)C(C)CN1C1CCN(C(=O)c2ccc(Cl)cc2)CC1]

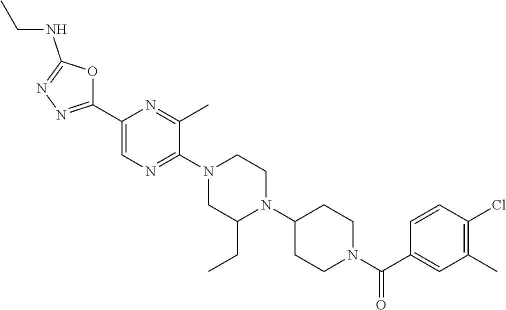 [SMILES: CCNc1nnc(-c2cnc(N3CCN(C4CCN(C(=O)c5ccc(Cl)c(C)c5)CC4)C(CC)C3)c(C)n2)o1]

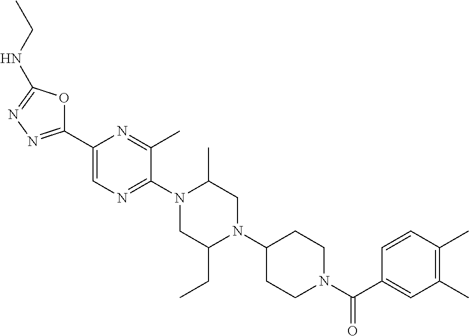 [SMILES: CCNc1nnc(-c2cnc(N3CC(CC)N(C4CCN(C(=O)c5ccc(C)c(C)c5)CC4)CC3C)c(C)n2)o1]

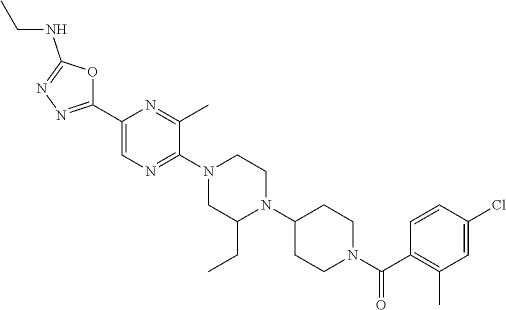 [SMILES: CCNc1nnc(-c2cnc(N3CCN(C4CCN(C(=O)c5ccc(Cl)cc5C)CC4)C(CC)C3)c(C)n2)o1]

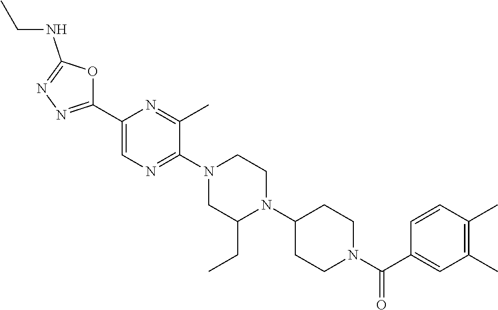 [SMILES: CCNc1nnc(-c2cnc(N3CCN(C4CCN(C(=O)c5ccc(C)c(C)c5)CC4)C(CC)C3)c(C)n2)o1]

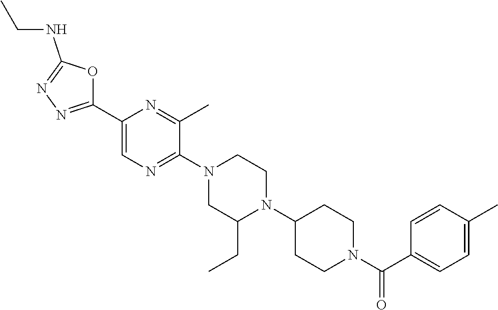 [SMILES: CCNc1nnc(-c2cnc(N3CCN(C4CCN(C(=O)c5ccc(C)cc5)CC4)C(CC)C3)c(C)n2)o1]

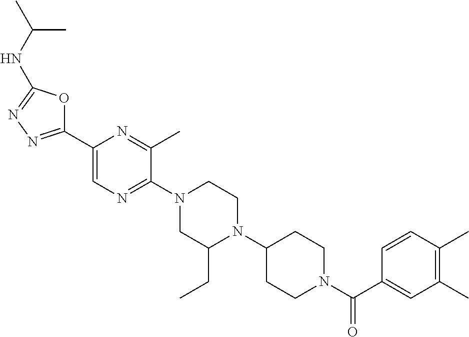 [SMILES: CCC1CN(c2ncc(-c3nnc(NC(C)C)o3)nc2C)CCN1C1CCN(C(=O)c2ccc(C)c(C)c2)CC1]

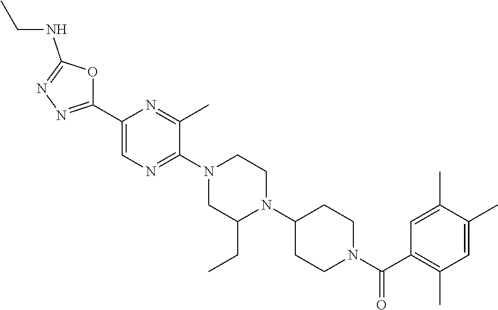 [SMILES: CCNc1nnc(-c2cnc(N3CCN(C4CCN(C(=O)c5cc(C)c(C)cc5C)CC4)C(CC)C3)c(C)n2)o1]

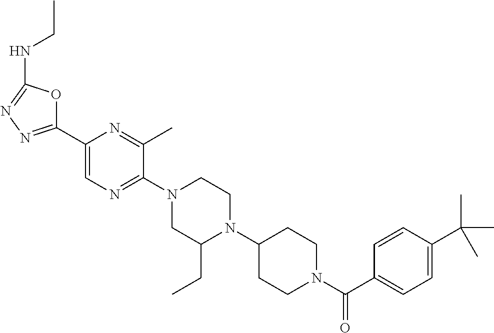 [SMILES: CCNc1nnc(-c2cnc(N3CCN(C4CCN(C(=O)c5ccc(C(C)(C)C)cc5)CC4)C(CC)C3)c(C)n2)o1]

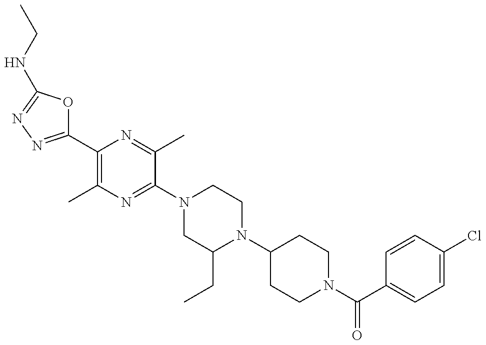 [SMILES: CCNc1nnc(-c2nc(C)c(N3CCN(C4CCN(C(=O)c5ccc(Cl)cc5)CC4)C(CC)C3)nc2C)o1]

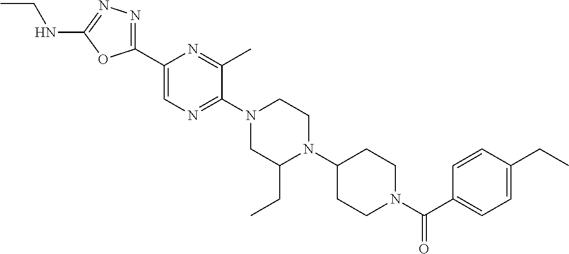 [SMILES: CCNc1nnc(-c2cnc(N3CCN(C4CCN(C(=O)c5ccc(CC)cc5)CC4)C(CC)C3)c(C)n2)o1]

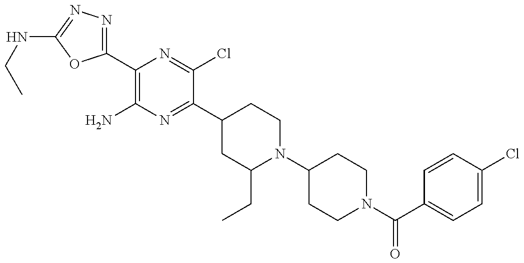 [SMILES: CCNc1nnc(-c2nc(Cl)c(C3CCN(C4CCN(C(=O)c5ccc(Cl)cc5)CC4)C(CC)C3)nc2N)o1]

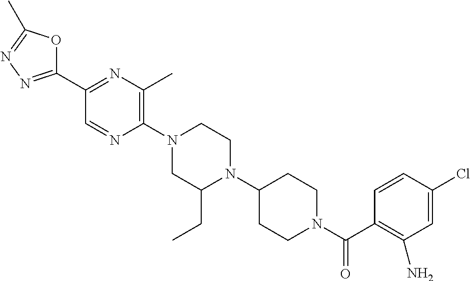 [SMILES: CCC1CN(c2ncc(-c3nnc(C)o3)nc2C)CCN1C1CCN(C(=O)c2ccc(Cl)cc2N)CC1]

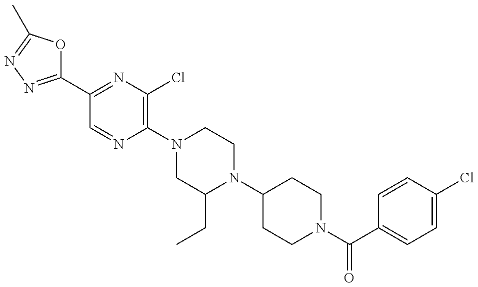 [SMILES: CCC1CN(c2ncc(-c3nnc(C)o3)nc2Cl)CCN1C1CCN(C(=O)c2ccc(Cl)cc2)CC1]

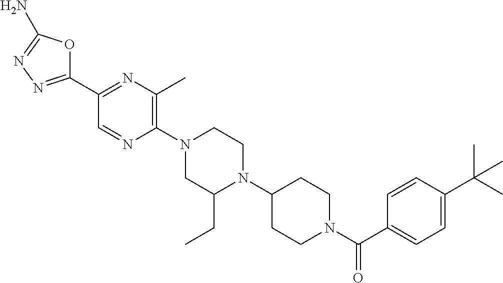 [SMILES: CCC1CN(c2ncc(-c3nnc(N)o3)nc2C)CCN1C1CCN(C(=O)c2ccc(C(C)(C)C)cc2)CC1]

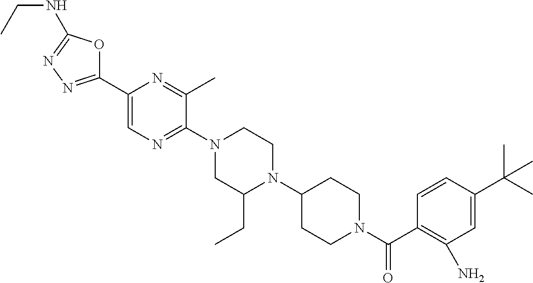 [SMILES: CCNc1nnc(-c2cnc(N3CCN(C4CCN(C(=O)c5ccc(C(C)(C)C)cc5N)CC4)C(CC)C3)c(C)n2)o1]

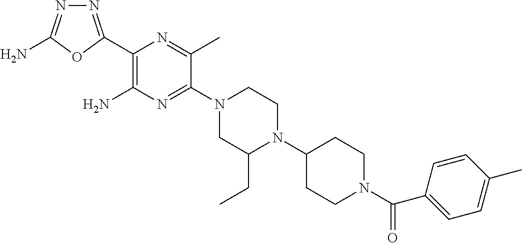 [SMILES: CCC1CN(c2nc(N)c(-c3nnc(N)o3)nc2C)CCN1C1CCN(C(=O)c2ccc(C)cc2)CC1]

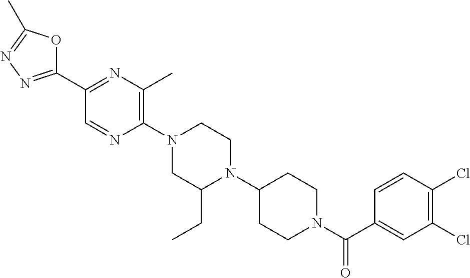 [SMILES: CCC1CN(c2ncc(-c3nnc(C)o3)nc2C)CCN1C1CCN(C(=O)c2ccc(Cl)c(Cl)c2)CC1]

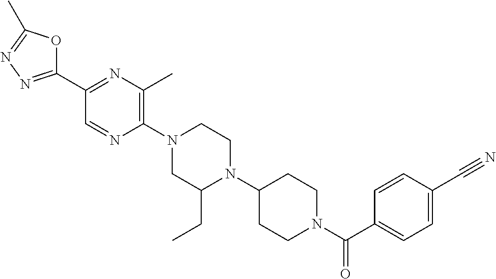 [SMILES: CCC1CN(c2ncc(-c3nnc(C)o3)nc2C)CCN1C1CCN(C(=O)c2ccc(C#N)cc2)CC1]

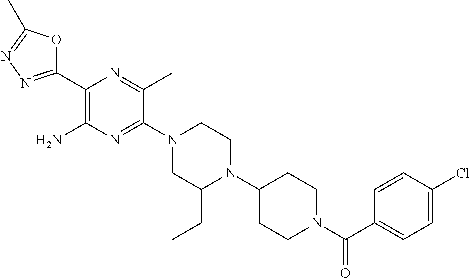 [SMILES: CCC1CN(c2nc(N)c(-c3nnc(C)o3)nc2C)CCN1C1CCN(C(=O)c2ccc(Cl)cc2)CC1]

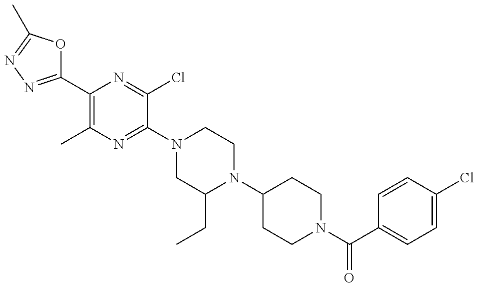 [SMILES: CCC1CN(c2nc(C)c(-c3nnc(C)o3)nc2Cl)CCN1C1CCN(C(=O)c2ccc(Cl)cc2)CC1]

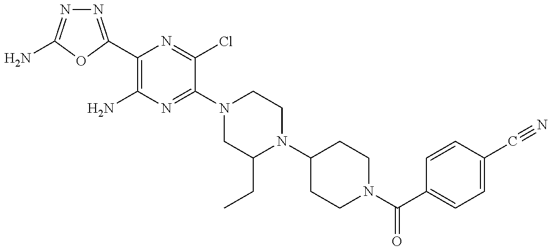 [SMILES: CCC1CN(c2nc(N)c(-c3nnc(N)o3)nc2Cl)CCN1C1CCN(C(=O)c2ccc(C#N)cc2)CC1]

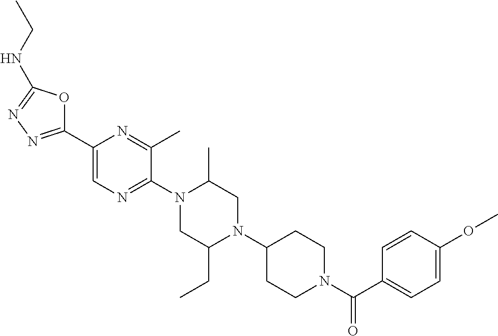 [SMILES: CCNc1nnc(-c2cnc(N3CC(CC)N(C4CCN(C(=O)c5ccc(OC)cc5)CC4)CC3C)c(C)n2)o1]

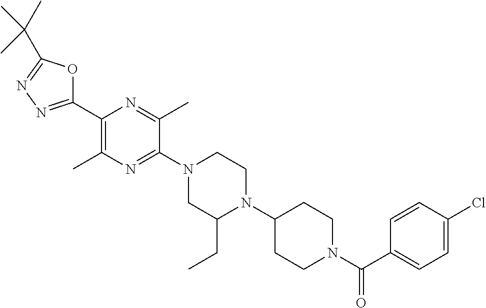 [SMILES: CCC1CN(c2nc(C)c(-c3nnc(C(C)(C)C)o3)nc2C)CCN1C1CCN(C(=O)c2ccc(Cl)cc2)CC1]

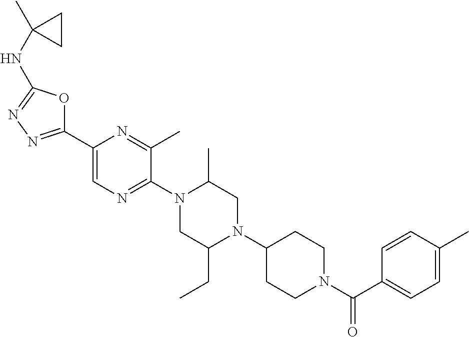 [SMILES: CCC1CN(c2ncc(-c3nnc(NC4(C)CC4)o3)nc2C)C(C)CN1C1CCN(C(=O)c2ccc(C)cc2)CC1]

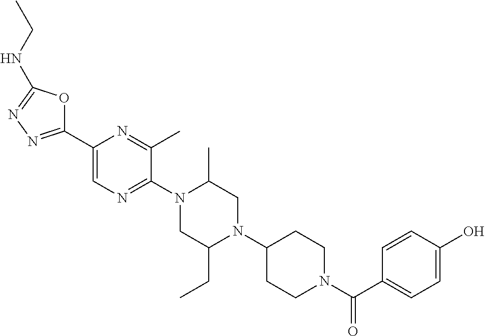 [SMILES: CCNc1nnc(-c2cnc(N3CC(CC)N(C4CCN(C(=O)c5ccc(O)cc5)CC4)CC3C)c(C)n2)o1]

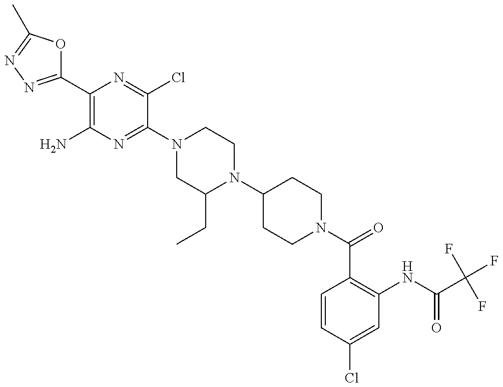 [SMILES: CCC1CN(c2nc(N)c(-c3nnc(C)o3)nc2Cl)CCN1C1CCN(C(=O)c2ccc(Cl)cc2NC(=O)C(F)(F)F)CC1]

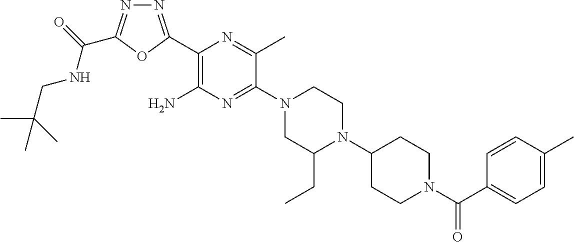 [SMILES: CCC1CN(c2nc(N)c(-c3nnc(C(=O)NCC(C)(C)C)o3)nc2C)CCN1C1CCN(C(=O)c2ccc(C)cc2)CC1]

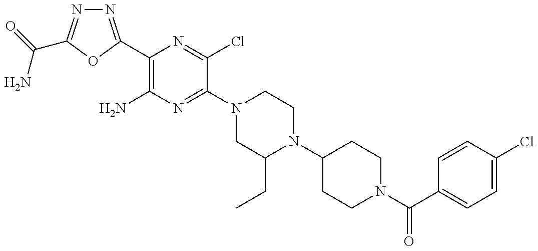 [SMILES: CCC1CN(c2nc(N)c(-c3nnc(C(N)=O)o3)nc2Cl)CCN1C1CCN(C(=O)c2ccc(Cl)cc2)CC1]

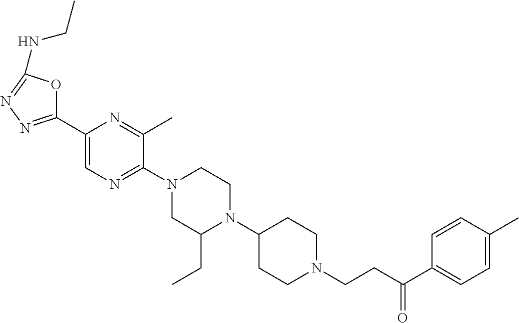 [SMILES: CCNc1nnc(-c2cnc(N3CCN(C4CCN(CCC(=O)c5ccc(C)cc5)CC4)C(CC)C3)c(C)n2)o1]

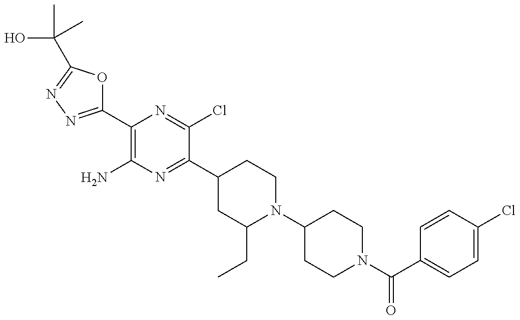 [SMILES: CCC1CC(c2nc(N)c(-c3nnc(C(C)(C)O)o3)nc2Cl)CCN1C1CCN(C(=O)c2ccc(Cl)cc2)CC1]

In some embodiments, the CXCR3 antagonist is T-487 (also known as AMG-487) or salts thereof. AMG-487 is an orally bioavailable chemokine CXCR3 antagonist that was tested in the clinic for psoriasis and rheumatoid arthritis. AMG 487 inhibits the binding of CXCL10 and CXCL11 to CXCR3 with IC₅₀s of 8.0 and 8.2 nM, respectively.

AMG-487 is chemically designated N-(1-(3-(4-ethoxyphenyl)-4-oxo-3,4-dihydropyrido[2,3-d]pyrimidin-2-yl)ethyl)-N-(pyridin-3-ylmethyl)-2-(4- (trifluoromethoxy)phenyl)acetamide, and its structure is shown below:

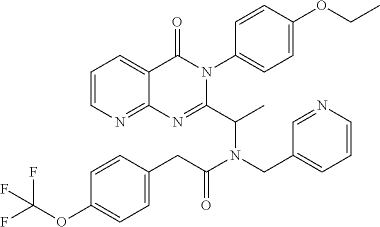 [SMILES: CCOc1ccc(-n2c(C(C)N(Cc3cccnc3)C(=O)Cc3ccc(OC(F)(F)F)cc3)nc3ncccc3c2=O)cc1]

T-487 and/or structurally related compounds are disclosed in WO 021083143 and WO 2006/02338, the contents of each of which are hereby incorporated by reference for all purposes and the specific purposes identified herein. In some embodiments, such compounds are represented by arty one or more of the structures shown in Table C2. Any one of the compounds depicted in Table C2 is suitable for use in the methods of the present disclosure.

TABLE C2
Compound

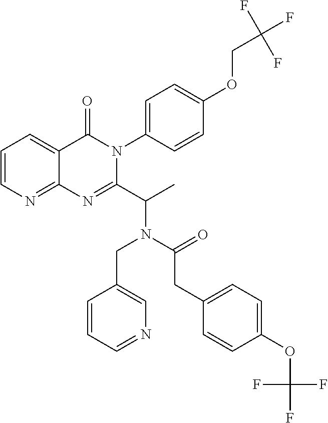 [SMILES: CC(c1nc2ncccc2c(=O)n1-c1ccc(OCC(F)(F)F)cc1)N(Cc1cccnc1)C(=O)Cc1ccc(OC(F)(F)F)cc1]

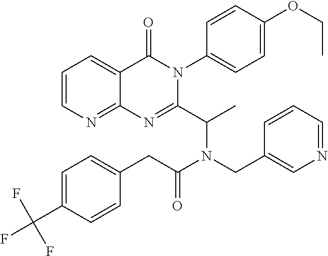 [SMILES: CCOc1ccc(-n2c(C(C)N(Cc3cccnc3)C(=O)Cc3ccc(C(F)(F)F)cc3)nc3ncccc3c2=O)cc1]

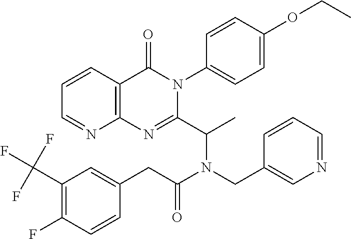 [SMILES: CCOc1ccc(-n2c(C(C)N(Cc3cccnc3)C(=O)Cc3ccc(F)c(C(F)(F)F)c3)nc3ncccc3c2=O)cc1]

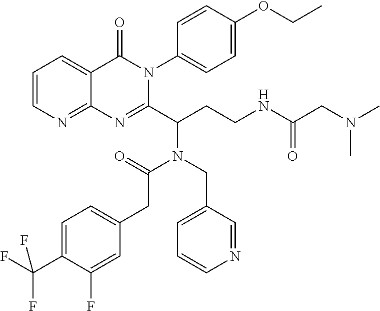 [SMILES: CCOc1ccc(-n2c(C(CCNC(=O)CN(C)C)N(Cc3cccnc3)C(=O)Cc3ccc(C(F)(F)F)c(F)c3)nc3ncccc3c2=O)cc1]

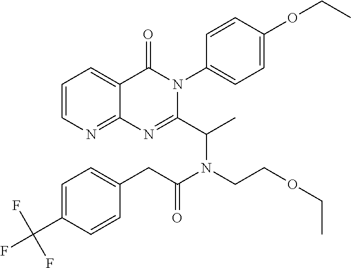 [SMILES: CCOCCN(C(=O)Cc1ccc(C(F)(F)F)cc1)C(C)c1nc2ncccc2c(=O)n1-c1ccc(OCC)cc1]

### Methods of Treatment

In certain embodiments, the compositions and methods of the present disclosure are useful for the prevention and/or treatment of symptoms of SARS-CoV-19 infections. In certain embodiments, the compositions and methods of the present disclosure are useful for the prevention and/or treatment of acute inflammatory responses. In certain embodiments, the compositions and methods of the present disclosure are useful for the prevention and/or treatment of acute inflammatory responses, e.g., cytokine storms that are associates with a coronavirus infection.

The present disclosure is based on the discovery that CXCR3 and CXCR4 antagonists may have therapeutic utility in the treatment of coronavirus symptoms, in particular in preventing cytokine storms in critical patients with coronavirus infections, in particular SARS-CoV-19.

Thus, in some embodiments, antagonists of CXCR3 and CXCR4 may prevent onset of severe SARS-CoV-19 symptoms. For example, CXCR3 or CXCR4 antagonists may prevent or ameliorate the hyper-inflammatory response in patients with SARS-CoV-19 pneumonia and prevent or ameliorate progress to cytokine storm. Successful intervention with a CXCR3 or CXCR4 antagonist may reduce life-threatening complications of SARS-CoV-19, including severe respiratory symptoms that often necessitate further medical intervention such as mechanical intervention.

Thus, in some embodiments, the present disclosure relates to a method of treating or alleviating at least one symptom of a coronavirus infection in a subject, by administering to the subject a therapeutically effective amount of a CXCR3 or CXCR4 antagonist. In some embodiments, the subject is a human.

In some embodiments, the symptom is fever. In other embodiments, the symptom is cough. In other embodiments, the symptom is dry cough. In other embodiments, the symptom is tiredness. In other embodiments, the symptom is sore throat. In other embodiments, the symptom is diarrhea. In other embodiments, the symptom is conjunctivitis. In other embodiments, the symptom is headache. In other embodiments, the symptom is loss of taste. In other embodiments, the symptom is loss of smell. In other embodiments, the symptom is a rash. In other embodiments, the symptom is difficulty breathing. In other embodiments, the symptom is shortness of breath. In other embodiments, the symptom is chest pain. In other embodiments, the symptom is chest pressure. In other embodiments, the symptom is Acute Respiratory Distress Syndrome (ARDS). In other embodiments, the symptom is organ failure. In other embodiments, the symptom is multiple organ failure. In other embodiments, the symptom is any combination of the foregoing.

In some embodiments, the present disclosure relates to a method of treating an acute inflammatory condition in a subject infected with a coronavirus, the method comprising the step of administering to the subject a therapeutically effective amount of a CXCR3 or CXCR4 antagonist. In some embodiments, the inflammatory condition comprises a cytokine storm. In some embodiments, the subject is a human.

In some embodiments, the present disclosure relates to a method of preventing a cytokine storm in a subject infected with a coronavirus, the method comprising the step of administering to the subject a therapeutically effective amount of a CXCR3 or CXCR4 antagonist. In some embodiments, the subject is a human.

In some embodiments, the present disclosure relates to a method of reducing or arresting viral load in a subject infected with a coronavirus, the method comprising the step of administering to the subject a therapeutically effective amount of a CXCR3 or CXCR4 antagonist. In some embodiments, the subject is a human.

Viral load can be measured by any viral diagnostic equipment or technique known in the art. A wide variety of samples can be used for virological testing. Such samples include, but are not limited to, upper respiratory swabs (nasopharyngeal swabs, nasopharyngeal wash/aspirate, oropharyngeal swabs, saliva) and lower respiratory specimens (sputum, bronchoalveolar lavage, lung tissue), as well as stool, rectal swabs, blood, skin, urine, semen, faeces, cerebrospinal fluid, tissue (e.g., biopsies), and the like. Techniques for measuring viral load include, but are not limited to, nucleic acid amplification-based tests (NATs) or non-nucleic acid-based tests. Examples of NATs include, but are not limited to, PCR (polymerase chain reaction), reverse transcription polymerase chain reaction (RT-PCR), and nucleic acid sequence-based amplification (NASBA). Viral load is typically reported as copies the virus in a milliliter (mL) of blood. Changes in viral load are usually reported as a log change (in powers of 10). For example, a three-log increase in viral load (3 log10) is an increase of 10³ or 1,000 times the previously reported level, while a drop from 500,000 to 500 copies would be a three-log-drop.

In one embodiment, the subject is infected with a coronavirus. In some embodiments, the coronavirus is selected from the group consisting of 229E (alpha coronavirus), NL63 (alpha coronavirus), OC43 (beta coronavirus), HKU1 (beta coronavirus), MERS-CoV (beta coronavirus that causes Middle East Respiratory Syndrome, or MERS), SARS-CoV (the beta coronavirus that causes severe acute respiratory syndrome, or SARS) SARS-CoV-2 (the novel coronavirus that causes coronavirus disease 2019, or COVID-19, also referred to herein as SARS-Covid-19). In some embodiments, the coronavirus is a severe acute respiratory syndrome coronavirus (SARS-CoV). In some embodiments, the coronavirus is a novel virus 2019-nCoV (SARS-CoV-19). In some embodiments, the coronavirus is a Middle East respiratory syndrome coronavirus (MERS-CoV). In one preferred embodiment, the coronavirus is SARS-CoV-19.

### Pharmaceutical Compositions

The present disclosure thus provides pharmaceutical compositions comprising CXCR3/CXCR4 antagonists and a pharmaceutically acceptable carrier. The compounds of the present disclosure can be formulated as pharmaceutical compositions and administered to a mammalian host, such as a human patient, in a variety of forms adapted to the chosen route of administration.

Routes of administration include, but are not limited to oral, topical, mucosal, nasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural and sublingual administration.

As used herein, the term “administering” generally refers to any and all means of introducing compounds described herein to the host subject. Compounds described herein may be administered in unit dosage forms and/or compositions containing one or more pharmaceutically-acceptable carriers, adjuvants, diluents, excipients, and/or vehicles, and combinations thereof.

As used herein, the terms “composition” generally refers to any product comprising more than one ingredient, including the compounds described herein. It is to be understood that the compositions described herein may be prepared from compounds described herein or from salts, solutions, hydrates, solvates, and other forms of the compounds described herein. It is appreciated that the compositions may be prepared from various amorphous, non-amorphous, partially crystalline, crystalline, and/or other morphological forms of the compounds described herein, and the compositions may be prepared from various hydrates and/or solvates of the compounds described herein. Accordingly, such pharmaceutical compositions that recite compounds described herein include each of, or any combination of, or individual forms of, the various morphological forms and/or solvate or hydrate forms of the compounds described herein.

In some embodiments, the CXCR3 or CXCR4 antagonists may be systemically (e.g., orally) administered in combination with a pharmaceutically acceptable vehicle such as an inert diluent or an assimilable edible carrier. For oral therapeutic administration, the active compound may be combined with one or more excipients and used in the form of ingestible tablets, buccal tablets, sublingual tablets, troches, capsules, elixirs, suspensions, syrups, wafers, and the like. The percentage of the compositions and preparations may vary and may be between about 1 to about 99% weight of the active ingredient(s) and excipients such as, but not limited to a binder, a filler, a diluent, a disintegrating agent, a lubricant, a surfactant, a sweetening agent; a flavoring agent, a colorant, a buffering agent, anti-oxidants, a preservative, chelating agents (e.g., ethylenediaminetetraacetic acid), and agents for the adjustment of tonicity such as sodium chloride.

Suitable binders include, but are not limited to, polyvinylpyrrolidone, copovidone, hydroxypropyl methylcellulose, starch, and gelatin.

Suitable fillers include, but are not limited to, sugars such as lactose, sucrose, mannitol or sorbitol and derivatives therefore (e.g. amino sugars), ethylcellulose, microcrystalline cellulose, and silicified microcrystalline cellulose.

Suitable diluents include, but are not limited to, dicalcium phosphate dihydrate, sugars, lactose, calcium phosphate, cellulose, kaolin, mannitol, sodium chloride, and dry starch.

Suitable disintegrants include, but are not limited to, pregelatinized starch, crospovidone, crosslinked sodium carboxymethyl cellulose and combinations thereof.

Suitable lubricants include, but are not limited to, sodium stearyl fumarate, stearic acid, polyethylene glycol or stearates, such as magnesium stearate.

Suitable surfactants or emulsifiers include, but are not limited to, polyvinyl alcohol (PVA), polysorbate, polyethylene glycols, polyoxyethylene- polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil.

Suitable flavoring agents and sweeteners include, but are not limited to, sweeteners such as sucralose and synthetic flavor oils and flavoring aromatics, natural oils, extracts from plants, leaves, flowers, and fruits, and combinations thereof. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot.

Suitable colorants include, but are not limited to, alumina (dried aluminum hydroxide), annatto extract, calcium carbonate, canthaxanthin, caramel, β-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide.

Suitable buffering or pH adjusting agent include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide and magnesium hydroxide.

Suitable tonicity enhancing agents include, but are not limited to, ionic and non-ionic agents such as, alkali metal or alkaline earth metal halides, urea, glycerol, sorbitol, mannitol, propylene glycol, and dextrose.

Suitable wetting agents include, but are not limited to, glycerin, cetyl alcohol, and glycerol monostearate.

Suitable preservatives include, but are not limited to, benzalkonium chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol, chlorohexidine, and polyhexamethylene biguanide.

Suitable antioxidants include, but are not limited to, sorbic acid, ascorbic acid, ascorbate, glycine, α-tocopherol, butylated hydroxyanisole (BHA), and butylated hydroxytoluene (BHT).

The CXCR3 or CXCR4 antagonists of the present disclosure may also be administered via infusion or injection (e.g., using needle (including microneedle) injectors and/or needle-free injectors). Solutions of the active composition can be aqueous, optionally mixed with a nontoxic surfactant and/or may contain carriers or excipients such as salts, carbohydrates and buffering agents (preferably at a pH of from 3 to 9), and, for some applications, they may be more suitably formulated as a sterile non-aqueous solution or as a dried form to be used in conjunction with a suitable vehicle such as sterile, pyrogen-free water or phosphate-buffered saline. For example, dispersions can be prepared in glycerol, liquid polyethylene glycols, triacetin, and mixtures thereof and in oils. The preparations may further contain a preservative to prevent the growth of microorganisms.

The pharmaceutical compositions may be formulated for parenteral administration (e.g., subcutaneous, intravenous, intra-arterial, transdermal, intraperitoneal or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Water is a preferred carrier when the pharmaceutical composition is administered intravenously. Saline solutions and aqueous dextrose and glycerol solutions can also be employed as liquid carriers, particularly for injectable solutions. Oils such as petroleum, animal, vegetable, or synthetic oils and soaps such as fatty alkali metal, ammonium, and triethanolamine salts, and suitable detergents may also be used for parenteral administration. Further, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. Suitable preservatives include e.g. sodium benzoate, benzoic acid, and sorbic acid. Suitable antioxidants include e.g. sulfites, ascorbic acid and □-tocopherol.

The preparation of parenteral compounds/compositions under sterile conditions, for example, by lyophilization, may readily be accomplished using standard pharmaceutical techniques well known to those skilled in the art.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. In one embodiment, the compositions are administered by the oral or nasal respiratory route for local or systemic effect. Compositions in pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, orally or nasally, from devices that deliver the formulation in an appropriate manner.

In yet another embodiment, the composition is prepared for topical administration, e.g. as an ointment, a gel, a drop, a patch or a cream. For topical administration to body surfaces using, for example, creams, gels, drops, ointments and the like, the compounds of the present disclosure can be prepared and applied in a physiologically acceptable diluent with or without a pharmaceutical carrier. Adjuvants for topical or gel base forms may include, for example, sodium carboxymethylcellulose, polyacrylates, polyoxyethylene-polyoxypropylene-block polymers, polyethylene glycol, wood wax alcohols, isostearic acid, cetyl alcohol, stearyl alcohol, white petrolatum, polysorbate 60, sorbitan monostearate, glycerin, xanthan gum, water, benzyl alcohol, methylparaben, and propylparaben. Additional additives may be selected from the group consisting of waxes, soaps, sorbitan esters, fatty acids, fatty acid esters, fatty acid oils, borates, cresol, chlorocresol, cellulose, methylcellulose, hydroxypropylcellulose, acacia, and the like. Examples of suitable topical dosage forms may be found in e.g., Tarun Garg, Goutam Rath & Amit K. Goyal (2015) Comprehensive review on additives of topical dosage forms for drug delivery, Drug Delivery, 22:8, 969-987, the contents of which are hereby incorporated by reference in their entirety.

Alternative formulations include nasal sprays, liposomal formulations, slow-release formulations, pumps delivering the drugs into the body (including mechanical or osmotic pumps) controlled-release formulations and the like, as are known in the art.

### Doses

As used herein, the term “therapeutically effective dose” means (unless specifically stated otherwise) a quantity of a compound which, when administered either one time or over the course of a treatment cycle affects the health, wellbeing or mortality of a subject (e.g., delays the onset of and/or reduces the severity of one or more of the symptoms associated with a coronavirus, e.g., SARS-Covid-19.

A CXCR3 or CXCR4 antagonist described herein can be present in a composition in an amount of about 0.001 mg, about 0.005 mg, about 0.01 mg, about 0.02 mg, about 0.03 mg, about 0.04 mg, about 0.05 mg, about 0.06 mg, about 0.07 mg, about 0.08 mg, about 0.09 mg about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 0.5 mg, about 10 mg, about 10.5 mg, about 11 mg, about 12 mg, about 12.5 mg, about 13 mg, about 13.5 mg, about 14 mg, about 14.5g, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg.

A CXCR3 or CXCR4 antagonist described herein described herein can be present in a composition in a range of from about 0.1 mg to about 100 mg; 0.1 mg to about 75 mg; from about 0.1 mg to about 50 mg; from about 0.1 mg to about 25 mg; from about 0.1 mg to about 10 mg; 0.1 mg to about 7.5 mg, 0.1 mg to about 5 mg; 0.1 mg to about 2.5 mg; from about 0.1 mg to about 1 mg; from about 0.5 mg to about 100 mg; from about 0.5 mg to about 75 mg; from about 0.5 mg to about 50 mg; from about 0.5 mg to about 25 mg; from about 0.5 mg to about 10 mg; from about 0.5mg to about 5 mg, from about 0.5mg to about 2.5 mg; from about 0.5 mg to about 1 mg; from about 1 mg to about 100 mg; from about 1 mg to about 75 mg; from about 0.1 mg to about 50 mg; from about 0.1 mg to about 25 mg; from about 0.1 mg to about 10 mg; from about 0.1 mg to about 5 mg; from about 0.1 mg to about 2.5 mg; from about 0.1 mg to about 1 mg.

### Dosing Regimens

The compounds described herein can be administered by any dosing schedule or dosing regimen as applicable to the patient and/or the condition being treated. Administration can be once a day (q.d.), twice a day (b.i.d.), thrice a day (t.i.d.), once a week, twice a week, three times a week, once every 2 weeks, once every three weeks, or once a month twice, and the like.

In some embodiments, the CXCR3 or CXCR4 antagonist is administered for a period of at least one day. In other embodiments, the CXCR3 or CXCR4 antagonist is administered for a period of at least 2 days. In other embodiments, the CXCR3 or CXCR4 antagonist is administered for a period of at least 3 days. In other embodiments, the CXCR3 or CXCR4 antagonist is administered for a period of at least 4 days. In other embodiments, the CXCR3 or CXCR4 antagonist is administered for a period of at least 5 days. In other embodiments, the CXCR3 or CXCR4 antagonist is administered for a period of at least 6 days. In other embodiments, the CXCR3 or CXCR4 antagonist is administered for a period of at least 7 days. In other embodiments, the CXCR3 or CXCR4 antagonist is administered for a period of at least 10 days. In other embodiments, the CXCR3 or CXCR4 antagonist is administered for a period of at least 14 days. In other embodiments, the CXCR3 or CXCR4 antagonist is administered for a period of at least one month. In some embodiments, the CXCR3 or CXCR4 antagonist is administered chronically for as long as the treatment is needed.

The present subject matter described herein will be illustrated more specifically by the following non-limiting examples, it being understood that changes and variations can be made therein without deviating from the scope and the spirit of the disclosure as hereinafter claimed. It is also understood that various theories as to why the disclosure works are not intended to be limiting.

Examples of CXCR3 or CXCR4 embodiments:
1. A method of treating or alleviating at least one symptom of a coronavirus infection in a subject, the method comprising the step of administering to the subject a therapeutically effective amount of a CXCR3 or CXCR4 antagonist.
2. The method according to embodiment 1, wherein the symptom is selected from the group consisting of fever, cough, tiredness, sore throat, diarrhea, conjunctivitis, headache, loss of taste, loss of smell, rash, difficulty breathing, shortness of breath, chest pain, chest pressure, Acute Respiratory Distress Syndrome (ARDS) and organ failure.
3. A method of preventing or treating an acute inflammatory condition in a subject infected with a coronavirus, the method comprising the step of administering to the subject a therapeutically effective amount of a CXCR3 or CXCR4 antagonist.
4. The method according to embodiment 3, wherein the inflammatory condition comprises a cytokine storm.
5. A method of preventing or treating a cytokine storm in a subject infected with a coronavirus, the method comprising the step of administering to the subject a therapeutically effective amount of a CXCR3 or CXCR4 antagonist.
6. A method of reducing or arresting viral load in a subject infected with a coronavirus, the method comprising the step of administering to the subject a therapeutically effective amount of a CXCR3 or CXCR4 antagonist.
7. The method according to any one of embodiments 1 to 6, wherein the coronavirus is selected from the group consisting of severe acute respiratory syndrome coronavirus (SARS-CoV), novel virus 2019-nCoV (SARS-CoV-19), and the Middle East respiratory syndrome coronavirus (MERS-CoV).
8. The method according to claim 7, wherein the corona virus is SARS-CoV-19.
9. The method according to any one of the preceding embodiments, wherein the CXCR3 or CXCR4 antagonist is selected from the group consisting of:
   (4-chlorophenyl)(4-(2-ethyl-4-(5-(5-(ethylamino)-1,3,4-oxadiazol-2-yl)-3-methylpyrazin-2-yl)-5-methylpiperazin-1-yl)piperidin-1-yl)methanone (PS-386113);
   N-(1-(3-(4-ethoxyphenyl)-4-oxo-3,4-dihydropyrido[2,3-d]pyrimidin-2-yl)ethyl)-N-(pyridin-3-ylmethyl)-2-(4-(trifluoromethoxy)phenyl)acetamide (AMG-487); and
   (2-(4-(6-amino-2-(((4-(((3-(cyclohexylamino)propyl)amino) methyl)cyclohexyl)methyl)amino)pyrimidin-4-yl)piperazin-1-yl)ethyl)phosphonic acid (Burixafor)
      and salts and any combinations thereof.
10. The method according to any one of embodiments 1-8, wherein the CXCR3 or CXCR4 antagonist is selected from the group consisting of a compound of any one of Table 1, Table 2, Table 3 and Table 4.
11. The method according to any one of the preceding embodiments, wherein the CXCR3 or CXCR4 antagonist is administered according to a dosing regimen selected from the group consisting of once daily (q.d.), twice daily (b.i.d.) thrice daily (t.i.d.), once a week, twice a week, three times a week, once every 2 weeks, once every three weeks, or once a month.
12. The method according to any one of the preceding embodiments, wherein the CXCR3 or CXCR4 antagonist is administered in a pharmaceutical composition, wherein the composition further comprises at least one pharmaceutically acceptable excipient.
13. The method according to embodiments 12, wherein the CXCR3 or CXCR4 antagonist is administered in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream.
14. The method according to embodiments 12 or 13, wherein the pharmaceutical composition is formulated for oral, topical, mucosal, intranasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural, sublingual oral, intranasal, intravenous, intraarterial, intrathecal, vaginal, rectal or subcutaneous administration.
15. The method according to any one of the preceding embodiments, wherein the subject is a human.
16. A pharmaceutical composition in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream, the composition comprising a CXCR3 or CXCR4 antagonist and at least one pharmaceutically acceptable excipient, wherein the CXCR3 or CXCR4 antagonist is selected from the group consisting of
   (4-chlorophenyl)(4-(2-ethyl-4-(5-(5-(ethylamino)-1,3,4-oxadiazol-2-yl)-3-methylpyrazin-2-yl)-5-methylpiperazin-1-yl)piperidin-1-yl)methanone (PS-386113);
   N-(1-(3-(4-ethoxyphenyl)-4-oxo-3,4-dihydropyrido[2,3-d]pyrimidin-2-yl)ethyl)-N-(pyridin-3-ylmethyl)-2-(4-(trifluoromethoxy)phenyl)acetamide (AMG-487);
and salts and any combinations thereof.
17. A pharmaceutical composition in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream, the composition comprising a CXCR3 or CXCR4 antagonist and at least one pharmaceutically acceptable excipient, wherein the CXCR3 or CXCR4 antagonist is selected from the group consisting of a compound of any one of Table 1, Table 2.

The present disclosure relates to methods of treating one or more symptoms of a coronavirus infection, particularly SARS-CoV-19. The present disclosure further relates to methods of treating or preventing an acute inflammatory response, e.g., a cytokine storm in a coronavirus patient, by administering a Toll-Like-Receptor (TLR) antagonist, in particular a TLR-7 or TLR-8 antagonist.

Toll-like receptors (TLRs) can recognize pathogens and are significantly expressed in immune cells. In particular, TLR-7 and TLR-8 are innate immune sensors that detect single stranded (ss) RNA from viruses such as SARS-Co-2. Activation of TLR 7/8 leads to immune cell activation and inflammation, which when not properly controlled can cause severed immunopathology. The present disclosure is based on the discovery that TLR-7 and TLR--8 antagonists may have therapeutic utility in the treatment of coronavirus symptoms, in particular in reducing inflammation and preventing cytokine storms in patients with coronavirus infections, in particular SARS-CoV-19.

Thus, in some embodiments, the present disclosure relates to a method of treating or alleviating at least one symptom of a coronavirus infection in a subject, by administering to the subject a therapeutically effective amount of a Toll-Like Receptor (TLR)-7 or TLR-8 antagonist. In some embodiments, the symptom is selected from the group consisting of fever, cough, tiredness, sore throat, diarrhea, conjunctivitis, headache, loss of taste, loss of smell, rash, difficulty breathing, shortness of breath, chest pain, chest pressure, Acute Respiratory Distress Syndrome (ARDS) and organ failure. In some embodiments, the subject is a human.

In some embodiments, the present disclosure relates to a method of treating an acute inflammatory condition in a subject infected with a coronavirus, the method comprising the step of administering to the subject a therapeutically effective amount of a Toll-Like Receptor (TLR)-7 or TLR-8 antagonist. In some embodiments, the inflammatory condition comprises a cytokine storm. In some embodiments, the subject is a human.

In some embodiments, the present disclosure relates to a method of preventing a cytokine storm in a subject infected with a coronavirus, the method comprising the step of administering to the subject a therapeutically effective amount of a Toll-Like Receptor (TLR)-7 or TLR-8 antagonist. In some embodiments, the subject is a human.

In some embodiments, the present disclosure relates to a method of reducing or arresting viral load in a subject infected with a coronavirus, the method comprising the step of administering to the subject a therapeutically effective amount of a Toll-Like Receptor (TLR)-7 or TLR-8 antagonist. In some embodiments, the subject is a human.

In some embodiments, the coronavirus is a severe acute respiratory syndrome coronavirus (SARS-CoV). In some embodiments, the coronavirus is a novel virus 2019-nCoV (SARS-CoV-19). In some embodiments, the coronavirus is a Middle East respiratory syndrome coronavirus (MERS-CoV). In one preferred embodiment, the coronavirus is SARS-CoV-19.

In some embodiments, the TLR-7 or TLR-8 antagonist is 2-(4-(2-(3,4-dimethoxyphenyl)-3-isopropyl-1H-indol-5-yl)piperidin-1-yl)-N-methylethan-1-amine or salts thereof. In other embodiments, the TLR-7 or TLR-8 antagonist is 5-(3-butylpyrrolidin-3-yl)-1H-indole or salts thereof. Combinations of TLR-7/8 antagonists may also be used in the methods of the present disclosure.

In some embodiments, the TLR-7 or TLR-8 antagonist is selected from the group consisting of a compound of any one of Table 1, Table 2 and Table 3.

In some embodiments, TLR-7 or TLR-8 antagonist is administered according to a dose regimen selected from the group consisting of once daily (q.d.), twice daily (b.i.d.) thrice daily (t.i.d.), once a week, twice a week, three times a week, once every 2 weeks, once every three weeks, or once a month.

In some embodiments, the TLR-7 or TLR-8 antagonist is administered in a pharmaceutical composition, wherein the composition further comprises at least one pharmaceutically acceptable excipient.

In some embodiments, the TLR-7 or TLR-8 antagonist is administered in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream.

In some embodiments, the pharmaceutical composition is formulated for oral, topical, mucosal, intranasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural, sublingual oral, intranasal, intravenous, intraarterial, intrathecal, vaginal, rectal or subcutaneous administration.

In some embodiments, the present disclosure relates to a topical pharmaceutical composition in a form selected from the group consisting of ointment, a gel, a drop, a patch and a cream, the composition comprising a TLR-7 or TLR-8 antagonist and at least one topically acceptable excipient, wherein the TLR-7 or TLR-8 antagonist is selected from the group consisting of 2-(4-(2-(3,4-dimethoxyphenyl)-3-isopropyl-1H-indol-5-yl)piperidin-1-yl)-N-methylethan-1-amine, 5-(3-butylpyrrolidin-3-yl)-1H-indole, and salts and combinations thereof.

In some embodiments, the present invention relates to topical pharmaceutical composition in a form selected from the group consisting of ointment, a gel, a drop, a patch and a cream, the composition comprising a TLR-7 or TLR-8 antagonist and at least one topically acceptable excipient, wherein the TLR-7 or TLR-8 antagonist is selected from the group consisting of a compound of any one of Table D1, Table D2 and Table D3.

### Toll-Like Receptor (TLR) Antagonists

Toll-like receptors (TLRs) can recognize pathogens and are significantly expressed in immune cells. In humans, the TLR family comprises ten members (TLR1-TLR10), which are expressed in innate immune cells such as macrophages as well as in epithelial and fibroblast cells. Activation of TLRs can be induced by a multitude of pathogen-associated molecular patterns (PAMPs) present in bacteria, viruses and other foreign organisms. TLRs play a major role in the initiation of innate immune responses, with the production of inflammatory cytokines, type 1 interferon (IFN) and other mediators. TLR activation causes nuclear translocation of the transcription factors NF-κB, IRF-3 and IRF-7, with production of innate pro-inflammatory cytokines (IL-1, IL-6, TNF-α) and type I IFN-α/β, which are essential for anti-viral responses. SARS-CoV-19 may prevent a successful immune response in infected individuals who progress to severe pathology via inhibition of the TNF-receptor-associated factors (TRAF) -3 and -6, which play an essential role in inducing interferon regulatory transcription factor (IRF)-3/7 in response to TLR-7 activation.

TLR-7 recognizes single-stranded RNA in endosomes, which is a common feature of viral genomes which are internalized by macrophages and dendritic cells. TLR-7 recognizes single-stranded RNA of viruses such as HIV and HCV. TLR-8 is an endosomal receptor that recognizes single stranded RNA (ssRNA), and can recognize ssRNA viruses such as Influenza, Sendai, and Coxsackie B viruses. TLR-8 binding to the viral RNA recruits MyD88 and leads to activation of the transcription factor NF-1(13 and an antiviral response. TLR-8 recognizes single-stranded RNA of viruses such as HIV and HCV. TLR 7/8 antagonists have been tested as possible therapeutics for autoimmune diseases, cancer and AIDS.

TLRs may be involved both in the initial failure of viral clearance and in the subsequent development of the deadly clinical manifestations of severe SARS-Cov-19, i.e., ARDS with fatal respiratory failure. In particular, TLR-7 and TLR-8 recognize viral single-stranded RNA and are therefore, likely to be implicated in clearance of SARS-CoV-19.

In some embodiments, TLR 7/8 antagonists can competitively inhibit the binding of spike protein/other viral pathogen-associated molecular pattern (PAMP) to TLR and dampen the expression of the proinflammatory cytokines like interleukin-1 (IL-1), IL-6, IL-8, and tumour necrosis factor-α.

For example, a compound for use in the methods of the present disclosure is a TLR-7 antagonist. A TLR-7 antagonist is characterized by the ability to inhibit the activation of a TLR-7 receptor, e.g., with an IC50 of 25 μM or less. By way of illustration, a TLR-7 antagonist inhibits the activation of a TLR-8 receptor with an IC50 of about 25 μM, 15 μM, 10 μM, 7.5 μM, 5 μM, 2.5 μM, 1.5 μM, 1 μM, 0.5 μM, 0.25 μM, 0.1 μM, 0.01 μM, or about 0.001 μM.

For example, a compound for use in the methods of the present disclosure is a TLR-8 antagonist. A TLR-8 antagonist is characterized by the ability to inhibit the activation of a TLR-8 receptor, e.g., with an IC50 of 25 μM or less. By way of illustration, a TLR-8 antagonist inhibits the activation of a TLR-8 receptor with an IC50 of about 25 μM, 15 μM, 10 μM, 7.5 μM, 5 μM, 2.5 μM, 1.5 μM, 1 μM, 0.5 μM, 0.25 μM, 0.1 μM, 0.01 μM, or about 0.001 μM.

For example, a compound for use in the methods of the present disclosure is a TLR7/8 antagonist. A TLR7/8 antagonist is characterized by the ability to inhibit, independently, the activation of both TLR-7 and TLR-8 receptors, e.g., with an IC50 of 25 μM or less. By way of illustration, a ILR7/8 antagonist inhibits the activation of both TLR-7 and TLR-8 receptors, independently, with an IC50 of about 25 μM, 15 μM, 10 μM, 7.5 μM, 5 μM, 2.5 μM, 1.5 μM, 1 μM, 0.5 μM, 0.25 μM, 0.1 μM, 0.01 μM, or about 0.001 μM.

In some embodiments, the compound is 2-(4-(2-3,4-dimethoxyphenyl)-3-isopropyl-1H-indol-5-yl)piperidin-1-yl)-N-methylethan-1-amine, the structure of which is represented below. The compound is a TLR-7/TLR-8 antagonist in clinical development for autoimmune diseases.

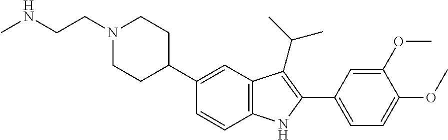

2-(4-(2-(3,4-dimethoxyphenyl)-3-isopropyl-1H-indol-5-yl)piperidin-1-methylethan-1-amine and/or structurally related compounds are described in PCT International Patent Applications WO 2018/026620 and WO 2019/126113, the contents of which are hereby incorporated by reference for all purposes and the specific purposes identified herein. in some embodiments, such compounds are represented by any one or more of the structures shown in Table 1. Any one of the compounds depicted in Table D1 is suitable for use in the methods of the present disclosure.

TABLE D1
Compound

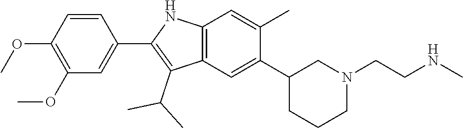

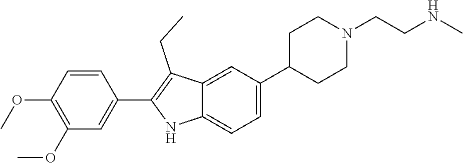

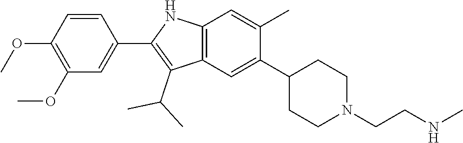

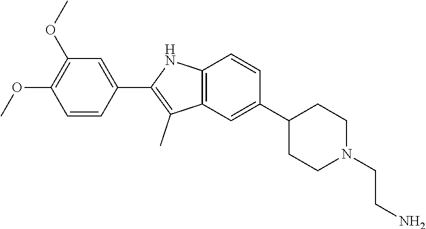 [SMILES: COc1ccc(-c2[nH]c3ccc(C4CCN(CCN)CC4)cc3c2C)cc1OC]

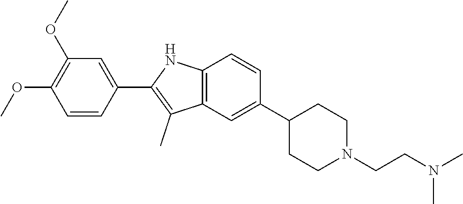

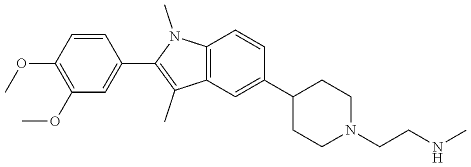

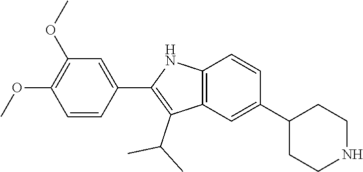 [SMILES: COc1ccc(-c2[nH]c3ccc(C4CCNCC4)cc3c2C(C)C)cc1OC]

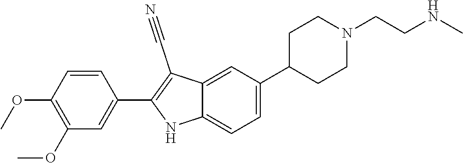

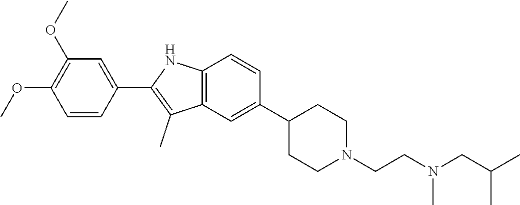 [SMILES: COc1ccc(-c2[nH]c3ccc(C4CCN(CCN(C)CC(C)C)CC4)cc3c2C)cc1OC]

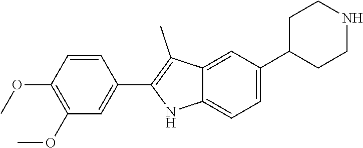 [SMILES: COc1ccc(-c2[nH]c3ccc(C4CCNCC4)cc3c2C)cc1OC]

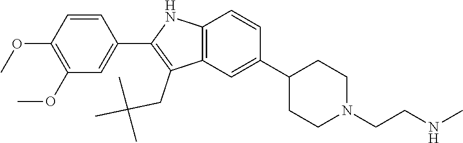

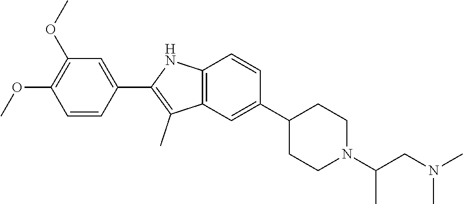

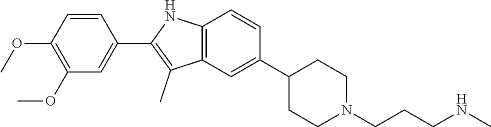

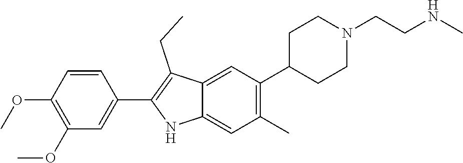

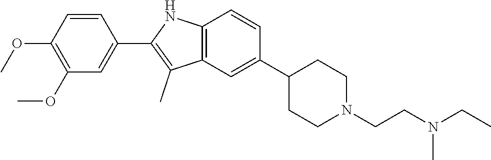 [SMILES: CCN(C)CCN1CCC(c2ccc3[nH]c(-c4ccc(OC)c(OC)c4)c(C)c3c2)CC1]

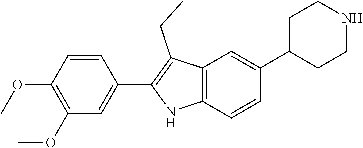 [SMILES: CCc1c(-c2ccc(OC)c(OC)c2)[nH]c2ccc(C3CCNCC3)cc12]

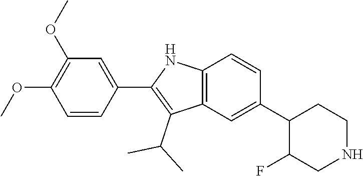 [SMILES: COc1ccc(-c2[nH]c3ccc(C4CCNCC4F)cc3c2C(C)C)cc1OC]

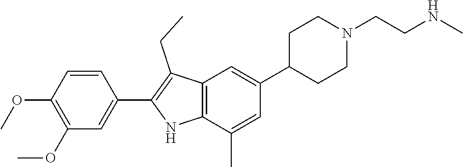

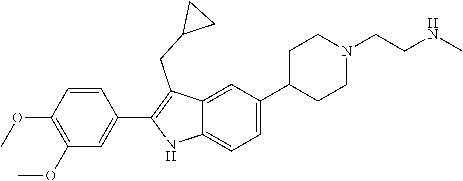

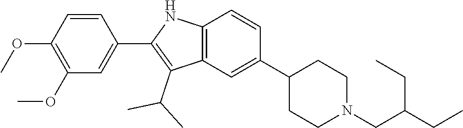 [SMILES: CCC(CC)CN1CCC(c2ccc3[nH]c(-c4ccc(OC)c(OC)c4)c(C(C)C)c3c2)CC1]

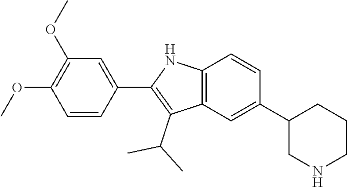 [SMILES: COc1ccc(-c2[nH]c3ccc(C4CCCNC4)cc3c2C(C)C)cc1OC]

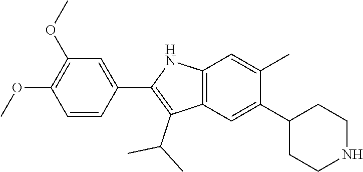 [SMILES: COc1ccc(-c2[nH]c3cc(C)c(C4CCNCC4)cc3c2C(C)C)cc1OC]

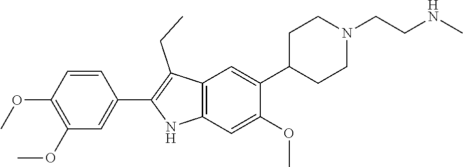

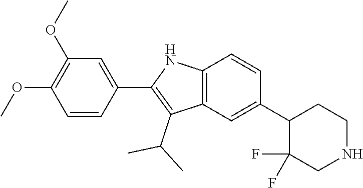 [SMILES: COc1ccc(-c2[nH]c3ccc(C4CCNCC4(F)F)cc3c2C(C)C)cc1OC]

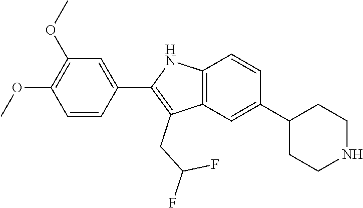 [SMILES: COc1ccc(-c2[nH]c3ccc(C4CCNCC4)cc3c2CC(F)F)cc1OC]

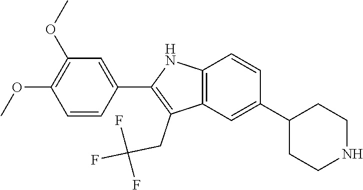 [SMILES: COc1ccc(-c2[nH]c3ccc(C4CCNCC4)cc3c2CC(F)(F)F)cc1OC]

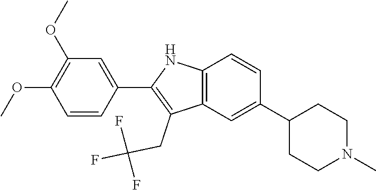

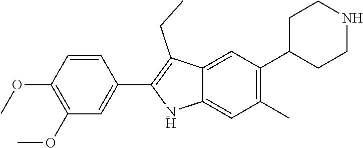 [SMILES: CCc1c(-c2ccc(OC)c(OC)c2)[nH]c2cc(C)c(C3CCNCC3)cc12]

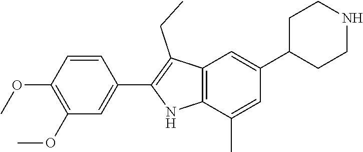 [SMILES: CCc1c(-c2ccc(OC)c(OC)c2)[nH]c2c(C)cc(C3CCNCC3)cc12]

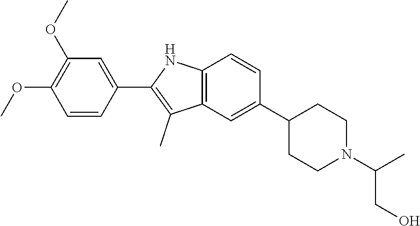 [SMILES: COc1ccc(-c2[nH]c3ccc(C4CCN(C(C)CO)CC4)cc3c2C)cc1OC]

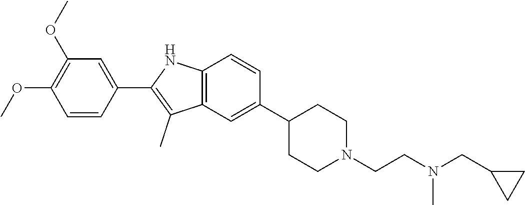 [SMILES: COc1ccc(-c2[nH]c3ccc(C4CCN(CCN(C)CC5CC5)CC4)cc3c2C)cc1OC]

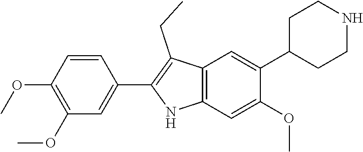 [SMILES: CCc1c(-c2ccc(OC)c(OC)c2)[nH]c2cc(OC)c(C3CCNCC3)cc12]

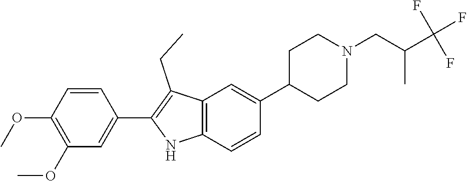 [SMILES: CCc1c(-c2ccc(OC)c(OC)c2)[nH]c2ccc(C3CCN(CC(C)C(F)(F)F)CC3)cc12]

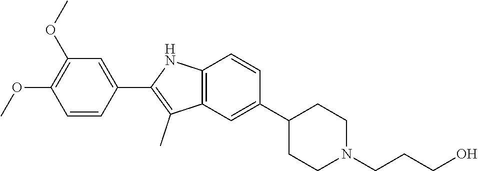 [SMILES: COc1ccc(-c2[nH]c3ccc(C4CCN(CCCO)CC4)cc3c2C)cc1OC]

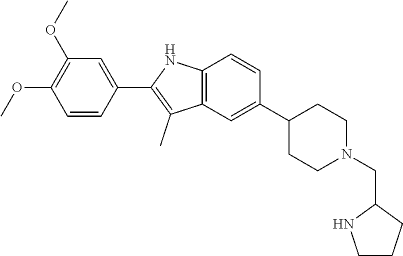 [SMILES: COc1ccc(-c2[nH]c3ccc(C4CCN(CC5CCCN5)CC4)cc3c2C)cc1OC]

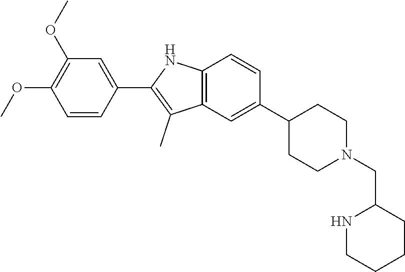 [SMILES: COc1ccc(-c2[nH]c3ccc(C4CCN(CC5CCCCN5)CC4)cc3c2C)cc1OC]

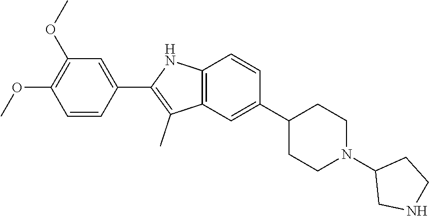 [SMILES: COc1ccc(-c2[nH]c3ccc(C4CCN(C5CCNC5)CC4)cc3c2C)cc1OC]

In some embodiments, the compound is a TLR-7 TLR-8 antagonist that is described in WO 2012/097173 and WO 2012/097177, the contents of each of which are hereby incorporated by reference for all purposes and the specific purposes identified herein. in some embodiments, such compounds are represented by any one or more of the structures shown in Table D2. Any one of the compounds depicted in Table 2 is suitable for use in the methods of the present disclosure.

TABLE D2

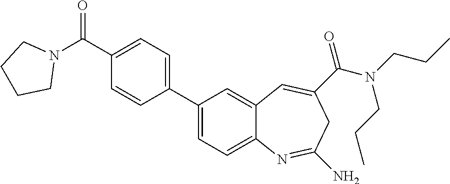 [SMILES: CCCN(CCC)C(=O)C1=Cc2cc(-c3ccc(C(=O)N4CCCC4)cc3)ccc2N=C(N)C1]

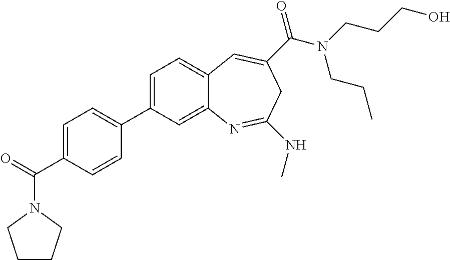 [SMILES: CCCN(CCCO)C(=O)C1=Cc2ccc(-c3ccc(C(=O)N4CCCC4)cc3)cc2N=C(NC)C1]

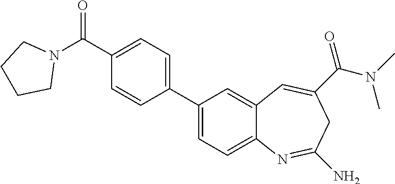 [SMILES: CN(C)C(=O)C1=Cc2cc(-c3ccc(C(=O)N4CCCC4)cc3)ccc2N=C(N)C1]

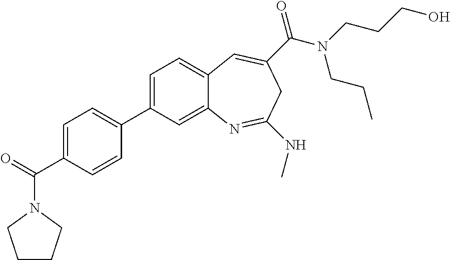 [SMILES: CCCN(CCCO)C(=O)C1=Cc2ccc(-c3ccc(C(=O)N4CCCC4)cc3)cc2N=C(NC)C1]

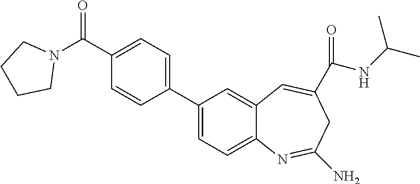 [SMILES: CC(C)NC(=O)C1=Cc2cc(-c3ccc(C(=O)N4CCCC4)cc3)ccc2N=C(N)C1]

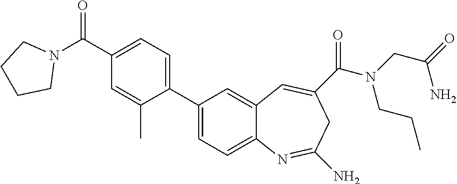 [SMILES: CCCN(CC(N)=O)C(=O)C1=Cc2cc(-c3ccc(C(=O)N4CCCC4)cc3C)ccc2N=C(N)C1]

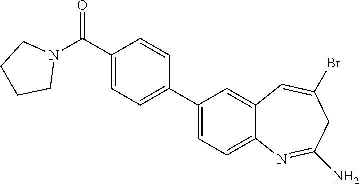 [SMILES: NC1=Nc2ccc(-c3ccc(C(=O)N4CCCC4)cc3)cc2C=C(Br)C1]

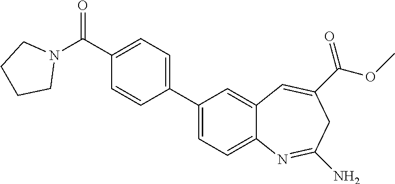 [SMILES: COC(=O)C1=Cc2cc(-c3ccc(C(=O)N4CCCC4)cc3)ccc2N=C(N)C1]

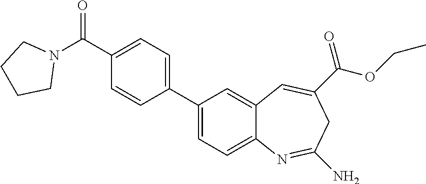 [SMILES: CCOC(=O)C1=Cc2cc(-c3ccc(C(=O)N4CCCC4)cc3)ccc2N=C(N)C1]

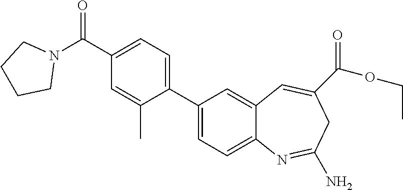 [SMILES: CCOC(=O)C1=Cc2cc(-c3ccc(C(=O)N4CCCC4)cc3C)ccc2N=C(N)C1]

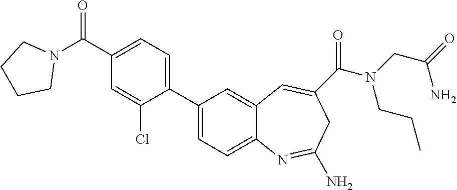 [SMILES: CCCN(CC(N)=O)C(=O)C1=Cc2cc(-c3ccc(C(=O)N4CCCC4)cc3Cl)ccc2N=C(N)C1]

In some embodiments, the compound is RG-7166, a TLR-7 antagonist which is chemically designated 5-(3-butylpyrrolidin-3-yl)-1H-indole. RG-7166 was previously in Phase 1 clinical development, and is represented by the structure:

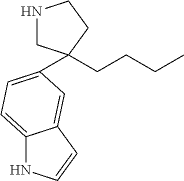 [SMILES: CCCCC1(c2ccc3[nH]ccc3c2)CCNC1]

Compounds structurally related to RG-7166 are described in PCT International Patent Application WO 2019/126113, the contents of which are hereby incorporated by reference for all purposes and the specific purposes identified herein. In some embodiments, such compounds are represented by any one or more of the structures shown in Table D3. disclosure.

TABLE D3
Compound

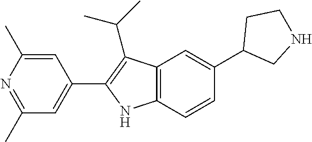 [SMILES: Cc1cc(-c2[nH]c3ccc(C4CCNC4)cc3c2C(C)C)cc(C)n1]

### Methods of Treatment

In certain embodiments, the compositions and methods of the present disclosure are useful for the prevention and/or treatment of symptoms of SARS-CoV-19 infections. In certain embodiments, the compositions and methods of the present disclosure are useful for the prevention and/or treatment of acute inflammatory responses. In certain embodiments, the compositions and methods of the present disclosure are useful for the prevention and/or treatment of acute inflammatory responses, e.g., cytokine storms that are associates with a coronavirus infection.

The present disclosure is based on the discovery that TLR-7 and TLR-8 antagonists may have therapeutic utility in the treatment of coronavirus symptoms, in particular in preventing cytokine storms in critical patients with coronavirus infections, in particular SARS-CoV-19. TLR-7 and TLR-8 antagonists are regulators that inhibit or reduce activation of TLR-mediated cytokine cascades and check over-reactive uncontrolled adaptive immune response. TLR antagonists are generally modified TLR agonists that bind TLRs but fail to induce the signal transduction.

Thus, in some embodiments, antagonists of TLR-7 and TLR-8 may prevent onset of severe SARS-CoV-19 symptoms. For example, TLR-7 or TUR-8 antagonists may prevent or ameliorate the hyper-inflammatory response in patients with SARS-CoV-19 pneumonia and prevent or ameliorate progress to cytokine storm. Successful intervention with TLR-7 or TLR-8 antagonist may reduce life-threatening complications of SARS-CoV-19, including severe respiratory symptoms that often necessitate further medical intervention such as mechanical intervention.

Thus, in some embodiments, he present disclosure relates to a method of treating or alleviating at least one symptom of a coronavirus infection in a subject, by administering to the subject a therapeutically effective amount of a Toll-Like Receptor (TLR)-7 or TLR-8 antagonist. In some embodiments, the subject is a human.

In some embodiments, the symptom is fever. In other embodiments, the symptom is cough. In other embodiments, the symptom is dry cough. In other embodiments, the symptom is tiredness. In other embodiments, the symptom is sore throat. In other embodiments, the symptom is diarrhea. In other embodiments, the symptom is conjunctivitis. In other embodiments, the symptom is headache. In other embodiments, the symptom is loss of taste. In other embodiments, the symptom is loss of smell. In other embodiments, the symptom is a rash. In other embodiments, the symptom is difficulty breathing. In other embodiments, the symptom is shortness of breath. In other embodiments, the symptom is chest pain. In other embodiments, the symptom is chest pressure. In other embodiments, the symptom is Acute Respiratory Distress Syndrome (ARDS). In other embodiments, the symptom is organ failure. In other embodiments, the symptom is multiple organ failure. In other embodiments, the symptom is any combination of the foregoing.

In some embodiments, the present disclosure relates to a method of treating an acute inflammatory condition in a subject infected with a coronavirus, the method comprising the step of administering to the subject a therapeutically effective amount of a Toll-Like Receptor (TLR)-7 or TLR-8 antagonist. In some embodiments, the inflammatory condition comprises a cytokine storm. In some embodiments, the subject is a human.

In some embodiments, the present disclosure relates to a method of preventing a cytokine storm in a subject infected with a coronavirus, the method comprising the step of administering to the subject a therapeutically effective amount of a Toll-Like Receptor (TLR)-7 or TLR-8 antagonist. In some embodiments, the subject is a human.

In some embodiments, the present disclosure relates to a method of reducing or arresting viral load in a subject infected with a coronavirus, the method comprising the step of administering to the subject a therapeutically effective amount of a Toll-Like Receptor (TLR)-7 or TLR-8 antagonist. In some embodiments, the subject is a human.

Viral load can be measured by any viral diagnostic equipment or technique known in the art. A wide variety of samples can be used for virological testing. Such samples include, but are not limited to, upper respiratory swabs (nasopharyngeal swabs, nasopharyngeal wash/aspirate, oropharyngeal swabs, saliva) and lower respiratory specimens (sputum, bronchoalveolar lavage, lung tissue), as well as stool, rectal swabs, blood, skin, urine, semen, faeces, cerebrospinal fluid, tissue (e.g., biopsies), and the like. Techniques for measuring viral load include, but are not limited to, nucleic acid amplification-based tests (NATs) or non-nucleic acid-based tests. Examples of NATs include, but are not limited to, PCR (polymerase chain reaction), reverse transcription polymerase chain reaction (RT-PCR), and nucleic acid sequence-based amplification (NASBA). Viral load is typically reported as copies the virus in a milliliter (mL) of blood. Changes in viral load are usually reported as a log change (in powers of 10). For example, a three-log increase in viral load (3 log10) is an increase of 10³ or 1,000 times the previously reported level, while a drop from 500,000 to 500 copies would be a three-log-drop.

In one embodiment, the subject is infected with a coronavirus. In some embodiments, the coronavirus is selected from the group consisting of 229E (alpha coronavirus), NL63 (alpha coronavirus), OC43 (beta coronavirus), HKU1 (beta coronavirus), MERS-CoV (beta coronavirus that causes Middle East Respiratory Syndrome, or MERS), SARS-CoV (the beta coronavirus that causes severe acute respiratory syndrome, or SARS) SARS-CoV-2 (the novel coronavirus that causes coronavirus disease 2019, or COVID-19, also referred to herein as SARS-Covid-19). In some embodiments, the coronavirus is a severe acute respiratory syndrome coronavirus (SARS-CoV). In some embodiments, the coronavirus is a novel virus 2019-nCoV (SARS-CoV-19). In some embodiments, the coronavirus is a Middle East respiratory syndrome coronavirus (MERS-CoV). In one preferred embodiment, the coronavirus is SARS-CoV-19.

### Pharmaceutical Compositions

The present disclosure thus provides pharmaceutical compositions comprising TLR 7/TLR 8 antagonists and a pharmaceutically acceptable carrier. The compounds of the present disclosure can be formulated as pharmaceutical compositions and administered to a mammalian host, such as a human patient, in a variety of forms adapted to the chosen route of administration.

Routes of administration include, but are not limited to oral, topical, mucosal, nasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural and sublingual administration.

As used herein, the term “administering” generally refers to any and all means of introducing compounds described herein to the host subject. Compounds described herein may be administered in unit dosage forms and/or compositions containing one or more pharmaceutically-acceptable carriers, adjuvants, diluents, excipients, and/or vehicles, and combinations thereof.

As used herein, the terms “composition” generally refers to any product comprising more than one ingredient, including the compounds described herein. It is to be understood that the compositions described herein may be prepared from compounds described herein or from salts, solutions, hydrates, solvates, and other forms of the compounds described herein. It is appreciated that the compositions may be prepared from various amorphous, non-amorphous, partially crystalline, crystalline, and/or other morphological forms of the compounds described herein, and the compositions may be prepared from various hydrates and/or solvates of the compounds described herein. Accordingly, such pharmaceutical compositions that recite compounds described herein include each of, or any combination of, or individual forms of, the various morphological forms and/or solvate or hydrate forms of the compounds described herein.

In some embodiments, the TLR-7 or TLR-8 antagonists may be systemically (e.g., orally) administered in combination with a pharmaceutically acceptable vehicle such as an inert diluent or an assimilable edible carrier. For oral therapeutic administration, the active compound may be combined with one or more excipients and used in the form of ingestible tablets, buccal tablets, sublingual tablets, troches, capsules, elixirs, suspensions, syrups, wafers, and the like. The percentage of the compositions and preparations may vary and may be between about 1 to about 99% weight of the active ingredient(s) and excipients such as, but not limited to a binder, a filler, a diluent, a disintegrating agent, a lubricant, a surfactant, a sweetening agent; a flavoring agent, a colorant, a buffering agent, anti-oxidants, a preservative, chelating agents (e.g., ethylenediaminetetraacetic acid), and agents for the adjustment of tonicity such as sodium chloride.

Suitable binders include, but are not limited to, polyvinylpyrrolidone, copovidone, hydroxypropyl methylcellulose, starch, and gelatin.

Suitable fillers include, but are not limited to, sugars such as lactose, sucrose, mannitol or sorbitol and derivatives therefore (e.g. amino sugars), ethylcellulose, microcrystalline cellulose, and silicified microcrystalline cellulose.

Suitable diluents include, but are not limited to, dicalcium phosphate dihydrate, sugars, lactose, calcium phosphate, cellulose, kaolin, mannitol, sodium chloride, and dry starch.

Suitable disintegrants include, but are not limited to, pregelatinized starch, crospovidone, crosslinked sodium carboxymethyl cellulose and combinations thereof.

Suitable lubricants include, but are not limited to, sodium stearyl fumarate, stearic acid, polyethylene glycol or stearates, such as magnesium stearate.

Suitable surfactants or emulsifiers include, but are not limited to. polyvinyl alcohol (PVA), polysorbate, polyethylene glycols, polyoxyethylene- polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil.

Suitable flavoring agents and sweeteners include, but are not limited to, sweeteners such as sucralose and synthetic flavor oils and flavoring aromatics, natural oils, extracts from plants. leaves, flowers, and fruits, and combinations thereof. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot.

Suitable colorants include, but are not limited to, alumina (dried aluminum hydroxide), annatto extract, calcium carbonate, canthaxanthin, caramel, r3-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide.

Suitable buffering or pH adjusting agent include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide and magnesium hydroxide.

Suitable tonicity enhancing agents include, but are not limited to, ionic and non-ionic agents such as, alkali metal or alkaline earth metal halides, urea, glycerol, sorbitol, mannitol, propylene glycol, and dextrose.

Suitable wetting agents include, but are not limited to, glycerin, cetyl alcohol, and glycerol monostearate.

Suitable preservatives include, but are not limited to, benzalkonium chloride, benzoxonium chloride, thiomersal, phenylmercuric nitrate, phenylmercuric acetate, phenylmercuric borate, methylparaben, propylparaben, chlorobutanol, benzyl alcohol. phenyl alcohol, chlorohexidine, and polyhexamethylene biguanide.

Suitable antioxidants include, but are not limited to, sorbic acid, ascorbic acid, ascorbate, glycine, α-tocopherol, butylated hydroxyanisole (BHA), and butylated hydroxytoluene (BHT).

The TLR-7 or TLR-8 antagonists of the present disclosure may also be administered via infusion or injection (e.g., using needle (including microneedle) injectors and/or needle-free injectors). Solutions of the active composition can be aqueous, optionally mixed with a nontoxic surfactant and/or may contain carriers or excipients such as salts, carbohydrates and buffering agents (preferably at a pH of from 3 to 9), and, for some applications, they may be more suitably formulated as a sterile non-aqueous solution or as a dried form to be used in conjunction with a suitable vehicle such as sterile, pyrogen-free water or phosphate-buffered saline. For example, dispersions can be prepared in glycerol, liquid polyethylene glycols, triacetin, and mixtures thereof and in oils. The preparations may further contain a preservative to prevent the growth of microorganisms.

The pharmaceutical compositions may be formulated for parenteral administration (e.g., subcutaneous, intravenous, intra-arterial, transdermal, intraperitoneal or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Water is a preferred carrier when the pharmaceutical composition is administered intravenously. Saline solutions and aqueous dextrose and glycerol solutions can also be employed as liquid carriers, particularly for injectable solutions. Oils such as petroleum, animal, vegetable, or synthetic oils and soaps such as fatty alkali metal, ammonium, and triethanolamine salts, and suitable detergents may also be used for parenteral administration. Further, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. Suitable preservatives include e.g. sodium benzoate, benzoic acid, and sorbic acid. Suitable antioxidants include e.g. sulfites, ascorbic acid and □-tocopherol.

The preparation of parenteral compounds/compositions under sterile conditions, for example, by lyophilization, may readily be accomplished using standard pharmaceutical techniques well known to those skilled in the art.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. In one embodiment, the compositions are administered by the oral or nasal respiratory route for local or systemic effect. Compositions in pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, orally or nasally, from devices that deliver the formulation in an appropriate manner.

In yet another embodiment, the composition is prepared for topical administration, e.g. as an ointment, a gel, a drop, a patch or a cream. For topical administration to body surfaces using, for example, creams, gels, drops, ointments and the like, the compounds of the present disclosure can be prepared and applied in a physiologically acceptable diluent with or without a pharmaceutical carrier. Adjuvants for topical or gel base forms may include, for example, sodium carboxymethylcellulose, polyacrylates, polyoxyethylene-polyoxypropylene-block polymers, polyethylene glycol, wood wax alcohols, isostearic acid, cetyl alcohol, stearyl alcohol, white petrolatum, polysorbate 60, sorbitan monostearate, glycerin, xanthan gum, water, benzyl alcohol, methylparaben, and propylparaben. Additional additives may be selected from the group consisting of waxes, soaps, sorbitan esters, fatty acids, fatty acid esters, fatty acid oils, borates, cresol, chlorocresol, cellulose, methylcellulose, hydroxypropylcellulose, acacia, and the like. Examples of suitable topical dosage forms may be found in e.g., Tarun Garg, Goutam Rath & Amit K. Goyal (2015) Comprehensive review on additives of topical dosage forms for drug delivery, Drug Delivery, 22:8, 969-987, the contents of which are hereby incorporated by reference in their entirety.

Alternative formulations include nasal sprays, liposomal formulations, slow-release formulations, pumps delivering the drugs into the body (including mechanical or osmotic pumps) controlled-release formulations and the like, as are known in the art.

### Doses

As used herein, the term “therapeutically effective dose” means (unless specifically stated otherwise) a quantity of a compound which, when administered either one time or over the course of a treatment cycle affects the health, wellbeing or mortality of a subject (e.g., delays the onset of and/or reduces the severity of one or more of the symptoms associated with a coronavirus, e.g., SARS-Covid-19.

A TLR-7 or TLR-8 antagonist described herein can be present in a composition in an amount of about 0.001 mg, about 0.005 mg, about 0.01 mg, about 0.02 mg, about 0.03 mg, about 0.04 mg, about 0.05 mg, about 0.06 mg, about 0.07 mg, about 0.08 mg, about 0.09 mg about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 0.5 mg, about 10 mg, about 10.5 mg, about 11 mg, about 12 mg, about 12.5 mg, about 13 mg, about 13.5 mg, about 14 mg, about 14.5g, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg.

A TLR-7 or TLR-8 antagonist described herein described herein can be present in a composition in a range of from about 0.1 mg to about 100 mg; 0.1 mg to about 75 mg; from about 0.1 mg to about 50 mg; from about 0.1 mg to about 25 mg; from about 0.1 mg to about 10 mg; 0.1 mg to about 7.5 mg, 0.1 mg to about 5 mg; 0.1 mg to about 2.5 mg; from about 0.1 mg to about 1 mg; from about 0.5 mg to about 100 mg; from about 0.5 mg to about 75 mg; from about 0.5 mg to about 50 mg; from about 0.5 mg to about 25 mg; from about 0.5 mg to about 10 mg; from about 0.5mg to about 5 mg, from about 0.5mg to about 2.5 mg; from about 0.5 mg to about 1 mg; from about 1 mg to about 100 mg; from about 1 mg to about 75 mg; from about 0.1 mg to about 50 mg; from about 0.1 mg to about 25 mg; from about 0.1 mg to about 10 mg; from about 0.1 mg to about 5 mg; from about 0.1 mg to about 2.5 mg; from about 0.1 mg to about 1 mg.

### Dosing Regimens

The compounds described herein can be administered by any dosing schedule or dosing regimen as applicable to the patient and/or the condition being treated. Administration can be once a day (q.d.), twice a day (b.i.d.), thrice a day (t.i.d.), once a week, twice a week, three times a week, once every 2 weeks, once every three weeks, or once a month twice, and the like.

In some embodiments, the TLR-7 or TLR-8 antagonist is administered for a period of at least one day. In other embodiments, the TLR-7 or TLR-8 antagonist is administered for a period of at least 2 days. In other embodiments, the TLR-7 or TLR-8 antagonist is administered for a period of at least 3 days. In other embodiments, the TLR-7 or TLR-8 antagonist is administered for a period of at least 4 days. In other embodiments, the TLR-7 or TLR-8 antagonist is administered for a period of at least 5 days. In other embodiments, the TLR-7 or TLR-8 antagonist is administered for a period of at least 6 days. In other embodiments, the TLR-7 or TLR-8 antagonist is administered for a period of at least 7 days. In other embodiments, the TLR-7 or TLR-8 antagonist is administered for a period of at least 10 days. In other embodiments, the TLR-7 or TLR-8 antagonist is administered for a period of at least 14 days. In other embodiments, the TLR-7 or TLR-8 antagonist is administered for a period of at least one month. In some embodiments, the TLR-7 or TLR-8 antagonist is administered chronically for as long as the treatment is needed.

The present subject matter described herein will be illustrated more specifically by the following non-limiting examples, it being understood that changes and variations can be made therein without deviating from the scope and the spirit of the disclosure as hereinafter claimed. It is also understood that various theories as to why the disclosure works are not intended to be limiting.

Examples of TLR-7 or TLR-8 antagonist embodiments:
1. A method of treating or alleviating at least one symptom of a coronavirus infection in a subject, the method comprising the step of administering to the subject a therapeutically effective amount of a Toil-Like Receptor (TLR)-7 or TLR-8 antagonist.
2. The method according to embodiment 1, wherein the symptom is selected from the group consisting of fever, cough, tiredness, sore throat, diarrhea, conjunctivitis, headache, loss of taste, loss of smell, rash, difficulty breathing, shortness of breath, chest pain, chest pressure, Acute Respiratory Distress Syndrome (ARDS) and organ failure.
3. A method of preventing or treating an acute inflammatory condition in a subject infected with a coronavirus, the method comprising the step of administering to the subject a therapeutically effective amount of a Toll-Like Receptor (TLR)-7 or TLR-8 antagonist.
4. The method according to embodiment 3, wherein the inflammatory condition comprises a cytokine storm.
5. A method of preventing or treating a cytokine storm in a subject infected with a coronavirus, the method comprising the step of administering to the subject a therapeutically effective amount of a Toll-Like Receptor (TLR)-7 or TLR-8 antagonist.
6. A method of reducing or arresting viral load in a subject infected with a coronavirus, the method comprising the step of administering to the subject a therapeutically effective amount of a Toll-Like Receptor (TLR)-7 or TLR-8 antagonist.
7. The method according to any one of embodiments 1 to 6, wherein the coronavirus is selected from the group consisting of severe acute respiratory syndrome coronavirus (SARS-CoV), novel virus 2019-nCoV (SARS-CoV-19), and the Middle East respiratory syndrome coronavirus (MERS-CoV).
8. The method according to embodiment 7, wherein the coronavirus is SARS-CoV-19.
9. The method according to any one of the preceding embodiments, wherein the TLR-7 or TLR-8 antagonist is selected from the group consisting of:
2-(4-(2-(3,4-dimethoxyphenyl)-3-isopropyl-1H-indol-5-yl)piperidin-1-yl)-N-methylethan-1-amine;
and salts and any combinations thereof.
10. The method according to any one of embodiments 1-8, wherein the TLR-7 or TLR-8 antagonist is selected from the group consisting of a compound of any one of Table 1, Table 2 and Table 3.
11. The method according to any one of the preceding embodiments, wherein the TLR-7 or TLR-8 antagonist is administered according to a dosing regimen selected from the group consisting of once daily (q.d.), twice daily (b.i.d.) thrice daily (t.i.d.), once a week, twice a week, three times a week, once every 2 weeks, once every three weeks, or once a month.
12. The method according to any one of the preceding embodiments, wherein the TLR-7 or TLR-8 antagonist is administered in a pharmaceutical composition, wherein the composition further comprises at least one pharmaceutically acceptable excipient.

13. The method according to embodiment 12, wherein the TLR-7 or TLR-8 antagonist is administered in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream.
14. The method according to embodiments 12 or 13, wherein the pharmaceutical composition is formulated for oral, topical, mucosal, intranasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural, sublingual oral, intranasal, intravenous, intraarterial, intrathecal, vaginal, rectal or subcutaneous administration.
15. The method according to any one of the preceding, embodiments, wherein the subject is a human.
16. A topical pharmaceutical composition in a form selected from the group consisting of ointment, a gel, a drop, a patch and a cream, the composition comprising a TLR-7 or TLR-8 antagonist and at least one topically acceptable excipient, wherein the TLR-7 or TLR-8 antagonist is selected from the group consisting of
2-(4-(2-(3,4-dimethoxyphenyl)-3-isopropyl-1H-indol-5-yl)piperidin-1-yl)-N-methylethan-1-amine;
and salts and any combinations thereof.
17. A topical pharmaceutical composition in a form selected from the group consisting of ointment, a gel, a drop, a patch and a cream, the composition comprising a TLR-7 or TLR-8 antagonist and at least one topically acceptable excipient, wherein the TLR-7 or TLR-8 antagonist is selected from the group consisting of a compound of any one of Table D1, Table D2 and Table D3.

Further embodiments and the full scope of applicability of the present disclosure will become apparent from the detailed description given hereinafter. However, it should be understood that the detailed description and specific examples, while indicating preferred embodiments of the disclosure, are given by way of illustration only, since various changes and modifications within the spirit and scope of the present disclosure will become apparent to those skilled in the art from this detailed description

The foregoing description of the specific embodiments will so fully reveal the general nature of the disclosure that others can, by applying knowledge within the skill of the relevant art(s) (including the contents of the documents cited herein, each of which is herein incorporated by reference in their respective entireties, and incorporated by reference herein for all purposes), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers. steps, operations, elements, and/or components, but do not preclude the presence or addition of one or more other features, integers, steps, operations, elements, components, and/or groups thereof.

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

1. A method of treating or alleviating at least one symptom of a coronavirus infection in a subject, the method comprising the step of administering to the subject a therapeutically effective amount of an angiotensin converting enzyme-2 (ACE2) modulator.

2. The method according to claim 1, wherein the symptom is selected from the group consisting of fever, cough, tiredness, sore throat, diarrhea, conjunctivitis, headache, loss of taste, loss of smell, rash, difficulty breathing, shortness of breath, chest pain, chest pressure, Acute Respiratory Distress Syndrome (ARDS) and organ failure.

3. (canceled)

4. The method according to claim 1, wherein the inflammatory condition comprises a cytokine storm.

5. (canceled)

6. (canceled)

7. The method according to claim 1, wherein the coronavirus is selected from the group consisting of severe acute respiratory syndrome coronavirus (SARS-CoV), novel virus 2019-nCoV (SARS-CoV-19), and the Middle East respiratory syndrome coronavirus (MERS-CoV).

8. The method according to claim 7, wherein the coronavirus is SARS-CoV-19.

9. The method according to claim 1, wherein the angiotensin converting enzyme-2 (ACE2) modulator is selected from the group consisting of:
(S,S)-2-(1-Carboxy-2-(3-(3,5-dichlorobenzyl)-3H-imidazol-4-yl)-ethylamino)-4-methylpentanoic acid (MLN-4760);
(2S)-2-acetamido-3-phenylpropanoic acid, or N-Acetyl-DL-phenytalanine (MR708);
(2S)-2-[[(2S,3R)-3-amino-2-hydroxy-4-phenylbutanoyl]amino]-4-methylpentanoic acid (Ubenimex);
(S)-2-amino-3-(4-boronophenyl)propanoic acid (Borofalan (10B);
(2-phenylacetyl)-L-glutamine (Antineoplaston AS2-5);
124-I-4-iodo-phenylalanine (124-I-TLX-101);
131-I-4-iodo-phenylalanine (131-I-TLX-101);
ethyl (2S)-2-[[2-(acetylsulfanylmethyl)-3-(2-methylphenyl)propanoyl]amino]-4-methylsulfanylbutanoate (SCH-42495);
(((S)-1-carboxy-5-((4-iodobenzyl)amino)pentyl)carbamoyl)-L-glutamic acid (Iofolastat-I 123);
8-guanidino-octanoyl-Asp-Phe (SC-49992);
(S)-5-amino-2-((1-propyl-1H-imidazol-4-yl)methyl)pentanoic acid (UK-369082);
4-{[2-(1H-imidazol-4-yl)ethyl]carbarnoyl} ibutanoic acid (Ingavirin®);
4-hydroxy-N,1-dimethyl-2-oxo-N-phenyl-3-quinolinecarboxamide (Roquinimex);
5-chloro-N-ethyl-4-hyroxy-1-methyl-2-oxo-N-phenyl-1,2-dihydroquinoline-3-carboxamide (Laquinimod);
4-hydroxy-5-methoxy-N,1-dimethyl-2-oxo-N-[4-(trifluoromethyl)phenyl]quinoline-3-carboxamide (Tasquinimod);
Acetyl-L-leucine;
(−)-N-[(trans-4-isopropylcyclohexane)carbonyl]-D-phenylalanine (Nateglinide);
S-(((R)-2-acetamido-2-carboxyethyl)thio)-N-acetyl-D-cysteine (N,N′-diacetyl-L-cystine);
2-Hydroxy-5-[(7-hydroxy-8-methyl-6-nitro-2-oxochromene-3-carbonyl)amino]benzoic acid (Nicousamide);
and salts and any combinations thereof.

10. The method according to claim 1, wherein the ACE2 modulator is selected from the group consisting of a compound of any one of Table 1, Table 2, Table 3, Table 4, Table 5, Table 6, Table 7 and Table 8.

11. The method according to claim 9, wherein the ACE2 modulator is administered according to a dosing regimen selected from the group consisting of once daily (q.d.), twice daily (b.i.d.) thrice daily (t.i.d.), once a week, twice a week, three times a week, once every 2 weeks, once every three weeks, or once a month.

12. The method according to claim 9, wherein the ACE2 modulator is administered in a pharmaceutical composition, wherein the composition further comprises at least one pharmaceutically acceptable excipient.

13. The method according to claim 12, wherein the ACE2 modulator is administered in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream.

14. The method according to claim 12, wherein the pharmaceutical composition is formulated for oral, topical, mucosal, intranasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural, sublingual oral, intranasal, intravenous, intraarterial, intrathecal, vaginal, rectal or subcutaneous administration.

15. The method according to claim 12, wherein the subject is a human.

16. The method according to claim 1, wherein the ACE2 modulator is an ACE2 inhibitor.

17. A pharmaceutical composition in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream, the composition comprising an ACE2 modulator and at least one pharmaceutically acceptable excipient, wherein the ACE2 modulator is selected from the group consisting of
(S,S)-2-(1-Carboxy-2-(3-(3,5-dichlorobenzyl)-3H-imidazol-4-yl)-ethylamino)-4-methylpentanoic acid (MLN-4760);
(2S)-2-acetamido-3-phenylpropanoic acid, or N-Acetyl-DL-phenylalanine (MR708);
(2S)-2-[[(2S,3R)-3-amino-2-hydroxy-4-phenylbutanoyl]amino]-4-methylpentanoic acid (Ubenimex);
(S)-2-amino-3-(4-boronophenyl)propanoic acid (Borofalan (10B);
(2-phenylacetyl)-L-glutamine (Antineoplaston AS2-5);
124-I-4-iodo-phenylalanine (124-I-TLX-101);
131-I-4-iodo-phenylalanine (131-I-TLX-101);
ethyl (2S)-2-[[2-(acetylsulfanylmethyl)-3-(2-methylphenyl)propanoyl]amino]-4-methylsulfanylbutanoate (SCH-42495);
(((S)-1-carboxy-5-((4-iodobenzyl)amino)pentyl)carbamoyl)-L-glutamic acid (Iofolastat-I 123);
8-guanidino-octanoyl-Asp-Phe (SC-49992);
(S)-5-amino-2-((1-propyl-1H-imidazol-4-yl)methyl)pentanoic acid (UK-369082);
4-{[2-(1H-imidazol-4-yl)ethyl]carbarnoyl}butanoic acid (Ingavirin®);
4-hydroxy-N,1-dimethyl-2-oxo-N-phenyl-3-quinolinecarboxamide (Roquinimex);
5-chloro-N-ethyl-4-hydroxy-1-methyl-2-oxo-N-phenyl-1,2-dihydroquinoline-3-carboxamide (Laquinimod);
4-hydroxy-5-methoxy-N,1-dimethyl-2-oxo-N-[4-(trifluoromethyl)phenyl]quinoline-3-carboxamide (Tasquinimod);
Acetyl-L-leucine;
(−)-N-[(trans-4-isopropylcyclohexane)carbonyl]-D-phenylalanine (Nateglinide);
S-(((R)-2-acetamido-2-carboxyethyl)thio)-N-acetyl-D-cysteine (N,N′-diacetyl-L-cystine);
2-Hydroxy-5-[(7-hydro-8-methyl-6-nitro-2-oxochromene-3-carbonyl)amino]benzoic acid (Nicousamide);
and salts and any combinations thereof.
and salts and any combinations thereof.

18. A pharmaceutical composition in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream, the composition comprising an ACE2 modulator and at least one pharmaceutically acceptable excipient, wherein the ACE2 modulator is selected from the group consisting of a compound of any one of Table 1, Table 2, Table 3, Table 4, Table 5, Table 6, Table 7 and Table 8.
